(19) United States
(12) Patent Application Publication
(10) Pub. No.: US 2024/0109962 A1
(43) Pub. Date: Apr. 4, 2024

(54) ANTIBODIES BINDING TO LEUKOCYTE IMMUNOGLOBULIN-LIKE RECEPTOR SUBFAMILY B MEMBER 2 (LILRB2) AND USES THEREOF

(71) Applicant: SPARX BIOSCIENCE LIMITED, ROAD TOWN (VG)

(72) Inventors: Qian CHEN, Mt. Prospect, IL (US); Jichun MA, Germantown, MD (US); Guidong ZHU, Gurnee, IL (US)

(21) Appl. No.: 18/476,226

(22) Filed: Sep. 27, 2023

Related U.S. Application Data
(60) Provisional application No. 63/377,416, filed on Sep. 28, 2022.

(51) Int. Cl. C07K 16/28; A61P 37/02; C12N 15/63

(52) U.S. Cl. CPC C07K 16/2803; A61P 37/02; C12N 15/63; C07K 2317/14; C07K 2317/24; C07K 2317/565; C07K 2317/567

(57) ABSTRACT

Antibodies that bind human leukocyte immunoglobulin-like receptor 2 (LILRB2) (anti-LILRB2 antibody) and uses thereof for modulating anti-tumor immune responses. Also provided herein are nucleic acid(s) encoding the anti-LILRB2 antibodies, host cells comprising the encoding nucleic acids and methods for producing the anti-LILRB2 antibodies.

### CROSS REFERENCE TO RELATED APPLICATIONS

This application claims the benefit of the filing dates of U.S. Provisional Application No. 63/377,416, filed Sep. 28, 2022, the entire contents of which are incorporated by reference herein.

### SEQUENCE LISTING

The instant application contains a Sequence Listing which has been filed electronically in XML format and is hereby incorporated by reference in its entirety. Said XML copy, created on Sep. 27, 2023, is named 112124-0025-70003US00_SEQ.XML and is 397,137 bytes in size.

### BACKGROUND OF THE INVENTION

The presence of tumor-associated myeloid cells is generally associated with poor prognosis in solid tumors. Tumor-associated myeloid cells maintain an immunosuppressive microenvironment within tumors and promote immune escape. There are two central groups of suppressive myeloid cells in the tumor microenvironment: tumor-associated macrophages (TAMs) and myeloid-derived suppressor cells (MDSCs).

Macrophages are highly plastic immune cells that dynamically integrate microenvironmental signals to shape their own functional phenotype. There are two main subsets of macrophages, M1 and M2, referring to classically activated macrophages and alternatively activated macrophages, respectively. M2 macrophages play immune suppressive roles in tumor microenvironment, while M1 macrophages immunostimulatory and cytotoxic effectors cells targeting tumor cells.

Most TAMs and MDSCs closely resemble M2 macrophages, which play suppressive roles in tumor microenvironment. Switching M2 macrophages to M1 subtype would be a promising approach to solid tumor treatment.

### SUMMARY OF THE INVENTION

The present disclosure is based, at least in part, on the development of antibodies that bind leukocyte immunoglobulin-like receptor 2 (LILRB2) (anti-LILRB2 antibody). Such antibodies have high binding specificity to human LILRB2 and are capable of blocking HLA-G and/or HLA-A2 from binding to LILRB2 receptor on cell surface, thereby reducing tumor immune evasion. As such, the anti-LILRB2 antibodies disclosed herein are expected to benefit tumor treatment.

In some instances, provided herein is an antibody that binds human leukocyte immunoglobulin-like receptor 2 (LILRB2) (anti-LILRB2 antibody). Such an anti-LILRB2 antibody comprises:
(a) a heavy chain variable region (V_{H}), which comprises
   (ai) a heavy chain CDR1 comprising GX₁SITSGYX₂WX₃ (SEQ ID NO: 1192), in which X₁ is Y or G, X₂ is Y, S, A, or H, and X₃ is N, S, or W;
   (aii) a heavy chain CDR2 comprising X₄ISYDGNX₅X₆ (SEQ ID NO: 194), in which X₄ is S, T, or Y, X₅ is A, I, K, L, M, N, P, S, T, V, or W and X₆ is H, L, N, S, or W; optionally wherein the heavy chain CDR2 comprises X₄ISYDGNX₅X₆YNPSLKN (SEQ ID NO: 416);
   (aiii) a heavy chain CDR3 comprising EEX₇TMX₈TTX₉WFX₁₀Y(SEQ ID NO: 196), in which X₇ is A or S, X₈ is F or I, X₉ is A or G, and X₁₀ is A, G, Q or S; and
(b) a light chain variable region (V_{L}), which comprises
   (bi) a light chain CDR1 comprising X₁₁X₁₂SEX₁₃IX₁₄SNX₁₅A (SEQ ID NO: 199), in which X₁₁ is E, G, L, N, Q, R, S, T or V, X₁₂ is A or G, X₁₃ is K, N, T, or V, X₁₄ is F or Y, and X₁₅ is L, N, or Q;
   (bii) a light chain CDR2 comprising GATX₁₆X₁₇X₁₈X₁₉ (SEQ ID NO: 201), in which X₁₆ is E, N, W, or Y, X₁₇ is F, H, L, P, or R, X₁₈ is A, N, R, or S, and X₁₉ is A, G, K, L, R, S, or V; and
   (biii) a light chain CDR3 comprising QX₂₀FWDX₂₁PYX₂₂ (SEQ ID NO: 203), in which X₂₀ is H or Q, X₂₁ is L, S, T, or Y, and X₂₂ is I, M, R, T, Y, or V.

In some embodiments, the X variables in the heavy chain CDRs may be: X₁ is Y, X₂ is Y, X₃ is N, X₄ is Y, X₅ is I, X₆ is N, X₇ is S, X₈ is I, X₉ is A, and/or X₁₀ is A.

Alternatively or in addition, the X variables in the light chain CDRs may be: X₁₁ is R, X₁₂ is A, X₁₃ is N, X₁₄ is Y, X₁₅ is L, X₁₆ is N, X₁₇ is L, X₁₈ is A, X₁₉ is S, X₂₀ is H, X₂₁ is Y, X₂₂ is T, or a combination thereof.

In some instances, the heavy chain CDR3 is EESTMITTAWFAY (SEQ ID NO: 11); and/or the light chain CDR3 is QHFWDYPYT (SEQ ID NO: 247).

In some examples, the anti-LILRB2 antibody may comprise the same heavy chain CDR1, CDR2, and CDR3, and the same light chain CDR1, CDR2, and CDR3 as the antibodies listed in Table 2.

In some embodiments, the anti-LILRB2 antibody comprises the same heavy chain CDRs as clone 2E1_FC11 (i.e., heavy chain CDR1, CDR2, and CDR3 set forth in SEQ ID NOs: 9, 243, and 11, respectively), and the same light chain CDRs as clone 2E1_FC11 (i.e., light chain CDR1, CDR2, and CDR3 set forth in SEQ ID NOs: 13, 14, and 247, respectively). In other embodiments, the heavy chain CDR1, CDR2, and CDR3 of the anti-LILRB2 antibody comprises up to five amino acid residue variations as relative to the heavy chain CDRs of clone 2E1_FC11 (SEQ IDs: 9, 243, and 11), and/or the light chain CDR1, CDR2, and CDR3 of the anti-LILRB2 antibody comprises up to five amino acid variations relative to the light chain CDRs of clone 2E1_FC11 (SEQ IDs: 13, 14, and 247).

In some embodiments, any of the anti-LILRB2 antibody provided herein can be a humanized antibody. For example, the V_{H} comprises (i) a V_{H} framework 1 (FR1) set forth as EVQLVESGGGLVQPGGSLRLSCAZiS (SEQ ID NO: 191), in which Z₁ is A or V, (ii) a V_{H} framework 2 (FR2) set forth as WZ₂RQAPGKGLEWVA (SEQ ID NO: 193), in which Z₂ is I or V, and/or (iii) a V_{H} framework 3 (FR3) set forth as RFTISRDZ₃SKNTLZ₄LQMNSLRAE DTAVYYCZ₅R (SEQ ID NO: 195), in which Z₃ is A, D or T, Z₄ is F, L, or V, and Z₅ is A or V, and/or (iv) a V_{H} framework 4 (FR4) set forth as WGQGTLVTVSS (SEQ ID NO: 197). Alternatively or in addition, the V_{L} comprises (i) a V_{L} FR1 set forth as comprises DIQZ₆TQSPSSLSASVGDRVTITC (SEQ ID NO: 198), in which Z₆ is L or M, (ii) a V_{L} FR2 set forth as WYQQKPGKAPKLLIY (SEQ ID NO: 200), (iii) a V_{L} FR3 set forth as GVPSRFSGSGSGTDZ₇TLTISSLQPEDFATYYC (SEQ ID NO: 202), in which Z₇ is F or Y, and/or (iv) a V_{L} FR4 set forth as FGQGTKVEIK (SEQ ID NO: 204).

Exemplary anti-LILRB2 antibodies provided herein are listed in Table 2, each of which is within the scope of the present disclosure.

In some examples, the V_{H} of the anti-LILRB2 antibody comprises an amino acid sequence at least 85% identical to the V_{H} of clone 2E1_FC11 (SEQ ID NO: 244); and/or wherein the V_{L} of the anti-LILRB2 antibody comprises an amino acid sequence at least 85% identical to the V_{L} of clone 2E1_FC11 (SEQ ID NO: 248). In specific examples, the V_{H} of the anti-LILRB2 antibody comprises the same V_{H} of 2E1_FC11 (comprising the amino acid sequence of SEQ ID NO: 244); and/or wherein the V_{L} of the anti-LILRB2 antibody comprises the same V_{L} of 2E1_FC11 (comprising the amino acid sequence of SEQ ID NO: 248).

Any of the anti-LILRB2 antibodies disclosed herein may be a full-length antibody or an antigen-binding fragment thereof.

In other aspects, provided herein is a nucleic acid or a set of nucleic acids, comprising a nucleotide sequence(s) encoding any of the anti-LILRB2 antibodies provided herein (e.g., clone 2E1_FC11 or a functional variant thereof). In some embodiments, the nucleic acid or set of nucleic acids can be a vector or a set of vectors comprising the nucleotide sequence(s) encoding the anti-LiLRB2 antibody. In some examples, the vector(s) can be an expression vector(s).

In addition, provided herein is a host cell or host cell set, comprising the nucleic acid(s) of encoding any of the anti-LILRB2 antibodies provided herein (e.g., clone 2E1_FC11 or a functional variant thereof). Such host cells may be a mammalian cell(s), a yeast cell(s), or a bacterial cell(s).

Further, the present disclosure features a pharmaceutical composition comprising (a) an anti-LILRB2 antibody as disclosed herein (e.g., clone 2E1_FC11 or a functional variant thereof), or a nucleic acid or set of nucleic acids encoding the antibody, and a pharmaceutically acceptable carrier.

In other aspects, the present disclosure features a method for modulating immune responses, comprising administering to a subject in need thereof an effective amount of the anti-LILRB2 antibody as disclosed herein (e.g., clone 2E1_FC11 or a functional variant thereof), the nucleic acid or set of nucleic acids encoding the antibody, or a pharmaceutical composition comprising the antibody or the encoding nucleic acid(s). In some instances, the subject is a human patient having or suspected of having cancer.

Moreover, the present disclosure provides a method for preparing an anti-LILRB2 antibody, comprising: culturing the host cell or host cell set carrying coding sequences for any of the anti-LILRB2 antibodies as disclosed herein (e.g., clone 2E1_FC11 or a functional variant thereof) under conditions allowing for expression of the antibody, and harvesting the antibody thus produced.

Additional embodiments are provided below, all of which are within the scope of the present disclosure.
Embodiment 1: an anti-LILRB2 antibody comprising: (a) a heavy chain variable region (V_{H}), which (i) comprises the same heavy chain complementarity determining region 1 (CDR1), complementarity determining region 2 (CDR2), and complementarity determining region 3 (CDR3) as those of a reference antibody; or (ii) comprises up to five amino acid variations in the CDR1, CDR2, and CDR3 collectively as compared with the reference antibody; and (b) a light chain variable region (V_{L}), which (i) comprises the same light chain CDR1, CDR2, and CDR3 as those of the reference antibody; or (ii) comprises up to five amino acid variations in the CDR1, CDR2, and CDR3 collectively as compared with the reference antibody. The reference antibodies include those listed in Table 1 below, e.g., 11B12, 9B6, 14F1, 10B11, 11D9, 4A9, 10D7, 13H5, 15A6, 6E8, 6H8, 13C4, 13D12, 2C1, 3C12, 7C12, 7G4, 8A4, 9H6, 12D8, 13C5, 15E2, 2F1, 3B5, 14C11, SLL-1, SLL-2, SLL-3, SLL-3, SLL-4, or SLL-5. In some instances, the anti-LILRB2 antibody comprises the V_{H} that comprises the same heavy chain CDRs as the reference antibody and the V_{L} that comprises the same light chain CDRs as the reference antibody.
Embodiment 2: the anti-LILRB2 antibody of Embodiment 1 is one of those listed in Table 1.
Embodiment 3: the anti-LILRB2 antibody of Embodiment 1 comprises the V_{H} that comprises up to five amino acid variations in heavy chain CDR1, CDR2, and CDR3 collective relative to reference antibody 11B12, and/or the V_{L} that comprises up to five amino acid variations in light chain CDR1, CDR2, and CDR3 collective relative to reference antibody 11B12.
Embodiment 4: the anti-LILRB2 antibody of Embodiment 3 comprises:
(ai) a heavy chain CDR1 comprising GX₁SITSGYX₂WX₃ (SEQ ID NO: 192), in which X₁ is Y or G, X₂ is Y, S, A, or H, and X₃ is N, S, or W;
(aii) a heavy chain CDR2 comprising X₄ISYDGNX₅X₆(SEQ ID NO: 194), in which X₄ is S, T, or Y, X₅ is A, I, K, L, M, N, P, S, T, V, or W and X₆ is H, L, N, S, or W; optionally wherein the heavy chain CDR2 comprises X₄ISYDGNX₅X₆YNPSLKN (SEQ ID NO: 416);
(aiii) a heavy chain CDR3 comprising EEX₇TMX₈TTX₉WFX₁₀Y (SEQ ID NO: 196), in which X₇ is A or S, X₈ is F or I, X₉ is A or G, and X₁₀ is A, G, Q or S;
(bi) a light chain CDR1 comprising X₁₁X₁₂SEX₁₃IX₁₄SNX₁₅A (SEQ ID NO: 199), in which X₁₁ is E, G, L, N, Q, R, S, T or V, X₁₂ is A or G, X₁₃ is K, N, T, or V, X₁₄ is F or Y, and X₁₅ is L, N, or Q;
(bii) a light chain CDR2 comprising GATX₁₆X₁₇X₁₈X₁₉ (SEQ ID NO: 201), in which X₁₆ is E, N, W, or Y, X₁₇ is F, H, L, P, or R, X₁₈ is A, N, R, or S, and X₁₉ is A, G, K, L, R, S, or V; and
(biii) a light chain CDR3 comprising QX₂₀FWDX₂₁PYX₂₂ (SEQ ID NO: 203), in which X₂₀ is H or Q, X₂₁ is L, S, T, or Y, and X₂₂ is I, M, R, T, Y, or V.
Embodiment 5: the anti-LILRB2 antibody of any one of Embodiments 1-4 is a humanized antibody.
Embodiment 6: the humanized antibody of Embodiment 5 may comprise a V_{H} that comprises a V_{H} framework 1 (FR1), a V_{H} framework 2 (FR2), a V_{H} framework 3 (FR3), and a V_{H} framework 4 (FR4) and a V_{L} that comprises a V_{L} FR1, a V_{L} FR2, a V_{L} FR3, and a V_{L} FR4, each of which may be set forth as follows:
   the V_{H} FR1 comprises EVQLVESGGGLVQPGGSLRLSCAZIS (SEQ ID NO: 191), in which Z₁ is A or V,
   the V_{H} FR2 comprises WZ₂RQAPGKGLEWVA (SEQ ID NO: 193), in which Z₂ is I or V,
   the V_{H} FR3 comprises RFTISRDZ₃SKNTLZ₄LQMNSLRAEDTAVYYCZ₅R (SEQ ID NO: 195), in which Z₃ is A, D or T, Z₄ is F, L, or V, and Z₅ is A or V, and the V_{H} FR4 comprises WGQGTLVTVSS (SEQ ID NO: 197); and
wherein:
   the V_{L} FR1 comprises DIQZ₆TQSPSSLSASVGDRVTITC (SEQ ID NO: 198), in which Z₆ is L or M,
   the V_{L} FR2 comprises WYQQKPGKAPKLLIY (SEQ ID NO: 200),
   the V_{L} FR3 comprises GVPSRFSGSGSGTDZ₇TLTISSLQPEDFATYYC (SEQ ID NO: 2020), in which Z₇ is F or Y, and
   the V_{L} FR4 comprises FGQGTKVEIK (SEQ ID NO: 204).
Embodiment 7: the anti-LILRB2 antibody of Embodiment 3 comprises the same heavy chain CDR1, CDR2, and CDR3, and the same light chain CDR1, CDR2, and CDR3 as the antibodies listed in Table 2.
Embodiment 8: the anti-LILRB2 antibody of Embodiment 7 may be any one of those listed in Table 2.
Embodiment 9: the anti-LILRB2 antibody of Embodiment 1 or 2 disclosed herein may comprise the same heavy chain CDRs and/or the same light chain CDRs as reference antibody 9B6.
Embodiment 10: the anti-LILRB2 antibody of Embodiment 8 may comprise the same heavy chain CDR1, CDR2, and CDR3, and the same light chain CDR1, CDR2, and CDR3 as the antibodies listed in Table 3.
Embodiment 11: the anti-LILRB2 antibody of any one of Embodiments 9-10 is a humanized antibody.
Embodiment 12: the humanized antibody of Embodiment 11 may comprise a V_{H} that comprises a V_{H} framework 1 (FR1), a V_{H} framework 2 (FR2), a V_{H} framework 3 (FR3), and a V_{H} framework 4 (FR4) and a V_{L} that comprises a V_{L} FR1, a V_{L} FR2, a V_{L} FR3, and a V_{L} FR4, each of which may set forth as follows:
   the V_{H} FR1 comprises EVQLVESGGZ₁LVQPGGSLRLSCAZ₂S (SEQ ID NO: 354), in which Z₁ is D or G, and Z₂ is A, T, or V,
   the V_{H} FR2 comprises WZ₃RQAPGKGLEWVA (SEQ ID NO: 193), in which Z₃ is I or V,
   the V_{H} FR3 comprises RZ₄TISRDZ₅SKNTLYLQMNSLRAEDTAVYYCZ₆R (SEQ ID NO: 355), in which Z₄ is A, F, I, L S, or V, Z₅ is D or N, and Z₆ is A or S, and
   the V_{H} FR4 comprises WGQGTLVTVSS (SEQ ID NO: 197); and
wherein:
   the V_{L} FR1 comprises DIQZ₇TQSPSSLSASVGDRVTITC (SEQ ID NO: 198), in which Z₇ is L or M,
   the V_{L} FR2 comprises WZ₈QQKPGKAPKLLIY (SEQ ID NO: 356), in which Z₈ is F or Y,
   the V_{L} FR3 comprises GVPSRFSGSGSGTDZ₉TLTISSLQPEDZ₁₀ATYYC (SEQ ID NO: 357), in which Z₉ is F or Y, and Zio is F or L; and
   the V_{L} FR4 comprises FGQGTKVEIK (SEQ ID NO: 204).
Embodiment 13: the anti-LILRB2 antibody of any one of Embodiments 9-12 is one of those listed in Table 3.
Embodiment 14: the anti-LILRB2 antibody of Embodiment 1 or Embodiment 2 disclosed herein may comprise the same heavy chain CDRs and/or the same light chain CDRs as reference antibody 14F1.
Embodiment 15: the anti-LILRB2 antibody of Embodiment 14 may comprise the same heavy chain CDR1, CDR2, and CDR3, and the same light chain CDR1, CDR2, and CDR3 as the antibodies listed in Table 4.
Embodiment 16: the anti-LILRB2 antibody of Embodiment 14 or Embodiment 15 is a humanized antibody.
Embodiment 17: the humanized antibody of Embodiment 16 may comprise a V_{H} that comprises a V_{H} framework 1 (FR1), a V_{H} framework 2 (FR2), a V_{H} framework 3 (FR3), and a V_{H} framework 4 (FR4) and a V_{L} that comprises a V_{L} FR1, a V_{L} FR2, a V_{L} FR3, and a V_{L} FR4, each of which may set forth as follows:
   the V_{H} FR1 comprises EVQLVESGGGLVQPGGSLRLSCAZiS (SEQ ID NO: 379), in which Z₁ is A, I, or V,
   the V_{H} FR2 comprises WZ₂RQAPGKGLEWVA (SEQ ID NO: 193), in which Z₂ is I or V,
   the V_{H} FR3 comprises RZ₃TISZ₄DZ₅SKZ₆TZ₇YLQMNSLRAEDTAVYYCAZ₈ (SEQ ID NO: 381), in which Z₃ is A, F, I, T, or V, Z₄ is K or R, Z₅ is D or N, Z₆ is A, L, or V, Z₇ is A, L, or V, and Z₈ is K or R, and
   the V_{H} FR4 comprises WGQGTLVTVSS (SEQ ID NO: 197); and
wherein:
   the V_{L} FR1 comprises DIQZ₉TQSPSSLSASVGDRVTITC (SEQ ID NO: 198), in which Z₉ is L or M,
   the V_{L} FR2 comprises WYQQKPGKAPKLLIY (SEQ ID NO: 200),
   the V_{L} FR3 comprises GVPSRFSGSGSGTDFTLTISSLQPEDZ₁₀ATYYC (SEQ ID NO: 383), in which Zio is F or L, and
   the V_{L} FR4 comprises FGQGTKVEIK (SEQ ID NO: 204).
Embodiment 18: the anti-LILRB2 antibody of Embodiment 14 is one of those listed in Table 4.
Embodiment 19: any of the anti-LILRB2 antibodies of Embodiments 1-18 disclosed herein may be a full-length antibody.
Embodiment 20: any of the anti-LILRB2 antibodies of Embodiments 1-18 disclosed herein may be an antigen-binding fragment thereof.
Embodiment 21: a nucleic acid or a set of nucleic acids, comprising a nucleotide sequence(s) encoding any of the anti-LILRB2 antibodies of any one of Embodiments 1-20 as disclosed herein.
Embodiment 21: the nucleic acid or the set of nucleic acids of Embodiment 21 is a vector or a set of vectors comprising the nucleotide sequence(s) encoding the anti-LILRB2 antibody.
Embodiment 22: the vector(s) of Embodiment 21 is an expression vector(s).
Embodiment 23: a host cell or host cell set, comprising the nucleic acid(s) encoding any of the anti-LILRB2 antibodies of Embodiments 1-20 as disclosed herein.
Embodiment 24: the host cell or host cell set of Embodiment 23 is a mammalian cell(s).
Embodiment 25: the host cell or host cell set of Embodiment 23 is a yeast cell(s).
Embodiment 26: the host cell or host cell set of Embodiment 23 is a bacterial cell(s).
Embodiment 27: a method for modulating immune responses, comprising administering to a subject in need thereof an effective amount of the anti-LILRB2 antibody of any one of Embodiments 1-20 disclosed herein, the nucleic acid or set of nucleic acids encoding the antibody, or a pharmaceutical composition comprising the antibody or the encoding nucleic acid(s).
Embodiment 28: the subject for treatment by the method of Embodiment 27 is a human patient having or suspected of having cancer.
Embodiment 29: a method for preparing an anti-LILRB2 antibody, comprising: (a) culturing the host cell or host cell set comprising a nucleic acid(s) encoding any of the anti-LILRB2 antibodies of Embodiments 1-20 as disclosed herein under conditions allowing for expression of the antibody, and (b) harvesting the antibody thus produced.

Also within the scope of the present disclosure includes a pharmaceutical composition comprising (a) an anti-LILRB2 antibody as disclosed herein, or a nucleic acid or set of nucleic acids encoding the antibody, and (b) a pharmaceutically acceptable carrier. Further, provided herein is such a pharmaceutical composition for use in treating cancer (e.g., a solid tumor) or any of the anti-LILRB2 antibodies disclosed herein for use in manufacturing a medicament for cancer treatment.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A and 1B include diagrams showing binding specificity of exemplary anti-LILRB2 antibodies to LILRB isoforms by ELISA. FIG. 1A: binding to LILRB1, LILRB2, and LILRB3 expressed on 293T cells. FIG. 1B: binding to His-tagged LILRB1, LILRB2, and LILRB3.

FIGS. 2A and 2B include diagrams showing binding specificity of exemplary anti-LILRB2 antibodies to human and cyno monkey LILRB2 by flow cytometry. FIG. 2A: binding to human LILRB1, human LILRB2, human LILRB3, and cyno monkey LILRB2 expressed on 293T cells. FIG. 2B: binding to human LILRB2 expressed on CHO-K1 cells.

FIGS. 3A-3F include diagrams showing blocking activities of exemplary anti-LILRB2 antibodies. FIGS. 3A-3B: blocking activity against binding of HLA-A2 to CHO-K1-huLILRB2 cells as indicated by MFI values (3A) and % of HLA-A2 petramer binding (3B). FIGS. 3C-3D: blocking activity against binding of HLA-G to CHO-K1-huLILRB2 cells as indicated by MFI values (3C) and % of HLA-G tetramer binding (3D). FIGS. 3E-3F: blocking activity against binding of ANGPTL2 to human LILRB2 as indicated by MFI values (3E) and % of LILRB2-Fc binding (3F).

FIGS. 4A and 4B include diagrams showing impact of exemplary anti-LILRB2 antibodies on cytokine secretion of PBMCs stimulated by LPS. FIG. 4A: enhanced TNFα expression. FIG. 4B: suppressed IL-10 expression.

FIGS. 5A-5C include diagrams showing binding activity of exemplary humanized anti-LILRB2 antibodies derived from parent clone 11B12 to 293T-LILRB2 cells. FIG. 5A: humanized clones h#2E1, h#2E2, h#2B1 and chimeric 11B12. FIG. 5B: humanized clones h#2E1, h#2D2, and H#2C1. FIG. 5C: humanized clones h#2E1, h#2D4, h#2D1, and h#2G6.

FIG. 6 is a diagram showing binding activity of exemplary humanized anti-LILRB2 antibody derived from parent clone 9B6 to 293T-LILRB2 cells.

FIGS. 7A-7C include diagrams showing blocking activities of exemplary humanized anti-LILRB2 antibodies against binding of HLA-G to 293T-LILRB2 cells. FIG. 7A: clones h#2E1, h#2E2, h#2B1, and h#2D2. FIG. 7B: clones h#2C1, h#2D4, h#2D1, and h#2G6. FIG. 7C: clones h#12a and chimeric 9B6.

FIG. 8 is a diagram showing blocking activities of exemplary humanized anti-LILRB2 antibody h#2E1 against binding of HLA-A2 to 293T-LILRB2 cells.

FIGS. 9A-9C include diagrams showing blocking activities of exemplary matured anti-LILRB2 antibodies against binding of HLA-G to 293T-LILRB2 cells. FIG. 9A: clones 2E1, 2E1_FC1, 2E1_FG9, 2E1_FD3, 2E1_FC3, 2E1_FF4, 2E1_FF5, and 2E1_FC11. FIG. 9B: clones 2E1_FC11, 2E1_FG8, 2E1_GB5, 2E1_GC6, 2E1_GB11, 2E1_GF1, 2E1_GD6, and 2E1_GH6. FIG. 9C: clones h#2E1, 2E1_GC6, 2E1_FG9, and 2E1_FC11.

FIG. 10 is a diagram showing blocking activities of exemplary matured anti-LILRB2 antibodies as indicated against binding of HLA-A2 to 293T-LILRB2 cells.

FIGS. 11A-11D include diagrams showing induction of macrophage M1 polarization by humanized anti-LILRB2 antibodies as indicated in a PBMC/LPS model. FIG. 11A: TNFα secretion. FIG. 11B: IL-10 secretion. FIG. 11C: TNFα secretion in culture of PBMCs treated by clone 2E1_FC11. FIG. 11B: IL-10 secretion in culture of PBMCs treated by clone 2E1_FC11.

FIG. 12 is a diagram showing induction of macrophage M1 polarization by clone 2E1_FC11 in human monocytes-derived macrophage as observed in an HMDM/LPS model.

FIGS. 13A-13D include diagrams showing activation of CD8+ T cells by clone 2E1_FC11 as compared with PD1 inhibitors. FIG. 13A: IFNγ secretion by immune cells treated with clone 2E1_FC11 as compared with PD1 inhibitor A105 (a single domain anti-PDL1 antibody). FIG. 13B: GM-CSF secretion by immune cells treated with clone 2E1_FC11 as compared with PD1 inhibitor A105. FIG. 13C: IFNγ secretion by immune cells treated with clone 2E1_FC11 as compared with PD1 inhibitor nivolumab. FIG. 13D: GM-CSF secretion by immune cells treated with clone 2E1_FC11 as compared with PD1 inhibitor nivolumab.

### DETAILED DESCRIPTION OF THE INVENTION

During tumor progression, tumor-associated myeloid cells may directly promote tumor cell proliferation and support epithelial-mesenchymal transition in tumors through the production of growth factors. At more advanced stages, the M2-like macrophage can be found in the metastatic cell niche where they promote tumor-initiated cell evasion of immune clearance. The mechanisms of immunosuppression employed by TAMs and MDSCs are mainly through inhibiting the activity of the adaptive immune system. Suppressive myeloid cells do so by either direct cell-cell interaction with target cells or through secreted factors. They could induce signaling via the immune checkpoint inhibitor PD1 and CTLA4 to induce T cell apoptosis and anergy. In addition, they can deprive the local environment of nutrients necessary for T-cell activation and function. Furthermore, they generate nitric oxygen, reactive nitrogen species, and reactive oxygen species to induce T-cell exhaustion. These mechanisms ultimately lead to a decrease in the effect and numbers of anti-tumor T-cells while enhancing the population of tumor-supporting regulatory T-cells. Targeting TAMs could significantly improve the efficacy of both conventional therapy and immunotherapy.

The remarkable functional plasticity of macrophages is the rationale for developing approaches to switch cells from M2-like immunosuppressive TAMs into M1-like immunostimulatory and antitumor cytotoxic effectors. The tumor-associated myeloid cells can be reprogrammed toward a pro-inflammatory state by direct intervention via small molecules and antibodies targeting key immune regulating receptors. Among them, the leukocyte immunoglobulin-like receptors (LILRs) play an important role in orchestrating immune responses in various immune cells. Two reprogramming strategies can be used—blocking a receptor that normally transduces an inhibitory intracellular signal or using an exogenous ligand to activate a receptor that stimulates proinflammatory intracellular cascades. LILRs include inhibitory receptor LILRB and activation receptors LILRA that regulate immune responses and inflammatory processes associated with disease progression. The LILRA and LILRB members are highly homologous in their extracellular regions and different in their intracellular regions. Six members of LILRA (LIRAs 1-6) associate with membrane adaptors to signal via immunoreceptor tyrosine-based activating motifs (ITAM), LILRB (LILRBs 1-5) members signal via multiple cytoplasmic immunoreceptor tyrosine-based inhibitory motif (ITIM). LILRs are expressed on various immune cells including NK, T, B, and myelomonocytic cells (monocytes, macrophages, dendritic cells, and granulocytes). The function of LILRBs in cancer has been extensively studied. Activation of LILRB signaling in cancer contributes to immune evasion and supports cancer development. The dual roles of LILRB1 and LIRB2 in cancer biology as immune checkpoint molecules and as tumor supporting factors suggest that LILRBs may present attractive targets for cancer treatment.

Among the LILRB family, LILRB2 is mainly expressed on myeloid cells and hematopoietic stem cells. It is a critical homeostatic surface regulator for myeloid cell maturation with great therapeutic value as a promising myeloid immune checkpoint target specifically aimed at myeloid cell functional determination. LILRB2 molecule composes four immunoglobulins (Ig)-like domains and a long cytoplasmic tail with ITIM motif. The ITIM-dependent recruitment of SHP1/SHP2 to LILRB2 negatively regulates immune activation. LILRB2 ligands include β2m-free forms of HLA class 1 (HLA-A, HLA-B, and HLA-C), non-classical MHC class 1 molecule (HLA-E, HLA-F, HLA-G, and HLA-H), and angiopoietin-like protein family (ANGPTL). LILRB2 on immune cells regulates cancer development through interaction with its ligands. HLA class 1 molecules that are aberrantly expressed in a variety of human malignant cells interact with LILRB2 expressed on immune cells. This interaction is involved in tumor immune evasion. HLA-G initially is selectively expressed at the maternal-fetal interface on cytotrophoblast cells and contributed to maternal-fetal tolerance. Numerous studies have been showing HLA-G gene transcription and protein translation is switched on in various tumor tissue and keep turning off in the surrounding normal area. HLA-G/LILRB pathways inhibit dendritic cells maturation and differentiation, promote macrophage differentiation into M2-like macrophages, and allow MDSC expansion. During macrophage maturation, LILRB2 antagonism inhibits AKT and STAT6 activation in response to M-CSF and IL4 treatment; and enhances NFκB and STAT1 activation in response to LPS/IFN-γ stimuli.

Provided herein are antibodies that bind LILRB2 (anti-LILRB2 antibodies), nucleic acids encoding such, methods for producing the anti-LILRB2 antibodies as disclosed herein and uses thereof for therapeutic, diagnostic, and/or research purposes.

### I. Anti-LILRB2 Antibodies

Leukocyte immunoglobulin-like receptor subfamily B member 2 (LILRB2) is a member of the leukocyte immunoglobulin-like receptor (LIR) family. LILRB2 belongs to the subfamily B class of the LIR family and contains two or four extracellular immunoglobulin domains, a transmembrane domain, and two to four cytoplasmic immunoreceptor tyrosine-based inhibitory motifs (ITIMs). Amino acid sequences of LILRB2 proteins are known in the art. As one example, the amino acid sequence of human LILRB2 can be found under GenBank accession no. NP_001074447.

Transcriptome analysis revealed that LILRB2 antagonism alters genes involved in cell cytoskeleton remodeling, lipid/cholesterol metabolism, and endosomal sorting pathways and changes differentiation gene networks to polarize TAMs toward an inflammatory phenotype. High expression of LILRB2 in DCs promotes DC tolerance, inhibits Th1 and CTL differentiation, and enhances the generation of type 2 cytokine secreting Th2 and Tc2 cells. LILRB2 on DCs diminishes the killing ability of CTLs by competitively binding to MHC-class 1 against CD8 or upregulating HLA-G in CTLs. These findings suggest that LILRB2 is a promising myeloid immune checkpoint target. LILRB2 antagonism through antibodies blocking ligand binding could reprogram and relieve the suppression of tumor-associated myeloid cells in the tumor microenvironment, thereby provoking antitumor immunity in cancer treatment.

The present disclosure provides antibodies that bind LILRB2 such as human LILRB2. In some embodiments, the anti-LILRB2 antibodies disclosed herein are capable of binding to LILRB2 expressed on cell surface. As such, the antibodies disclosed herein may be used for either therapeutic or diagnostic purposes to target LILRB2-positive cells (e.g., immune cells such as macrophages to enhance anti-tumor immune responses). As used herein, the term “anti-LILRB2 antibody” refers to any antibody capable of binding to a LILRB2 receptor (e.g., a LILRB2 receptor expressed on cell surface), for a fragment thereof, which can be of a suitable source, for example, human or a non-human mammal (e.g., mouse, rat, rabbit, primate such as monkey, etc.). In some instances, the anti-LILRB2 antibodies disclosed herein may be capable of binding to an extracellular domain of a LILRB2.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody”, e.g., anti-LILRB2 antibody, encompasses not only intact (e.g., full-length) polyclonal or monoclonal antibodies, but also antigen-binding fragments thereof (such as Fab, Fab′, F(ab′)2, Fv), single-chain antibody (scFv), fusion proteins comprising an antibody portion, humanized antibodies, chimeric antibodies, diabodies, single domain antibody (e.g., nanobody), single domain antibodies (e.g., a V_{H} only antibody), multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody, e.g., anti-LILRB2 antibody, includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs).

The anti-LILRB2 antibody described herein may be a full-length antibody, which contains two heavy chains and two light chains, each including a variable domain and a constant domain. Alternatively, the anti-LILRB2 antibody can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, C_{L} and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

The antibodies described herein can be of a suitable origin, for example, murine, rat, or human. Such antibodies are non-naturally occurring, i.e., would not be produced in an animal without human act (e.g., immunizing such an animal with a desired antigen or fragment thereof or isolated from antibody libraries). Any of the antibodies described herein, e.g., anti-LILRB2 antibody, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In some embodiments, the anti-LILRB2 antibodies are human antibodies, which may be isolated from a human antibody library or generated in transgenic mice. For example, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Amgen, Inc. (Fremont, Calif.) and HuMAb-Mouse™ and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the antibody library display technology, such as phage, yeast display, mammalian cell display, or mRNA display technology as known in the art can be used to produce human antibodies and antibody fragments in vitro, from immunoglobulin variable (V) domain gene repertoires from unimmunized donors.

In other embodiments, the anti-LILRB2 antibodies may be humanized antibodies or chimeric antibodies. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. In general, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, one or more Fv framework region (FR) residues of the human immunoglobulin are replaced by corresponding non-human residues.

Furthermore, the humanized antibody may comprise residues that are found neither in the recipient antibody nor in the imported CDR or framework sequences but are included to further refine and optimize antibody performance. In some instances, the humanized antibody may comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, or six) which are altered with respect to the original antibody, which are also termed one or more CDRs “derived from” one or more CDRs from the original antibody. Humanized antibodies may also involve affinity maturation. Methods for constructing humanized antibodies are also well known in the art. See, e.g., Queen et al., Proc. Natl. Acad. Sci. USA, 86:10029-10033 (1989).

In some embodiments, the anti-LILRB2 antibody disclosed herein can be a chimeric antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region. Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

In some embodiments, the anti-LILRB2 antibodies described herein specifically bind to the corresponding target antigen (e.g., human LILRB2) or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen (LILRB2 such as human LILRB2) or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen (i.e., only baseline binding activity can be detected in a conventional method).

In some examples, the anti-LILBR2 antibody disclosed herein specifically binds LILRB2. In some instances, the anti-LILBR2 antibody disclosed herein does not bind to another member of the LILRB family, such as LILRB1 or LILBR3 (i.e., no significant binding can be detected in a routine method). In other instances, the anti-LILBR2 antibody disclosed herein is capable of binding to another member of the LILRB family, such as LILRB1 or LILBR3, but at a much lower binding affinity relative to the binding affinity to LILRB2.

In some embodiments, an anti-LILRB2 antibody as described herein has a suitable binding affinity for the target antigen (e.g., human LILRB2) or antigenic epitopes thereof. As used herein, “binding affinity” refers to the apparent association constant or K_{A}. The K_{A} is the reciprocal of the dissociation constant (K_{D}). The anti-LILRB2 antibody described herein may have a binding affinity (K_{D}) of at least 100 nM, 10 nM, 1 nM, 0.1 nM, or lower for LILRB2 (e.g., human LILRB2). An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 90, 100, 500, 1000, 10,000 or 10⁵ folds. In some embodiments, any of the anti-LILRB2 antibodies may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

Any of the anti-LILRB2 antibody as described herein, e.g., the exemplary anti-LILRB2 antibodies provided here, can bind and inhibit (e.g., reduce or eliminate) the activity of LILRB2-positive cells (e.g., immune cells such as macrophages expressing LILRB2). In some embodiments, the anti-LILRB2 antibody as described herein can bind and inhibit the activity of LILRB2-positive cells (e.g., cancer cells) by at least 30% (e.g., 35%, 40%, 45%, 50%, 60%, 70%, 80%, 90%, 95% or greater, including any increment therein). The inhibitory activity of an anti-LILRB2 antibody described herein can be determined by routine methods known in the art, e.g., by an assay for measuring the K_{i,}^{app} value.

In some examples, the K_{i,}^{app} value of an antibody may be determined by measuring the inhibitory effect of different concentrations of the antibody on the extent of a relevant reaction; fitting the change in pseudo-first order rate constant (v) as a function of inhibitor concentration to the modified Morrison equation (Equation 1) yields an estimate of the apparent Ki value. For a competitive inhibitor, the Ki^{app} can be obtained from the y-intercept extracted from a linear regression analysis of a plot of K_{i,}^{app} versus substrate concentration.

$$v = A \cdot \frac{\begin{matrix}( [ E ] - [ I ] - {K}_{i}^{app} ) + \\ \sqrt{{( [ E ] - [ I ] - {K}_{i}^{app} )}^{2} + 4 [ E ] \cdot } {K}_{i}^{app}\end{matrix}}{2} \qquad ( Equation 1 )$$

Where A is equivalent to vₒ/E, the initial velocity (vₒ) of the enzymatic reaction in the absence of inhibitor (I) divided by the total enzyme concentration (E). In some embodiments, the anti-LILRB2 antibody described herein may have a Ki^{app} value of 1000, 500, 100, 50, 40, 30, 20, 10, 5 pM or less for the target antigen or antigen epitope.

Exemplary Anti-LILRB2 Antibodies

A number of exemplary anti-LILRB2 antibodies, are provided in Table 1 below (CDRs for each exemplary anti-LILRB2 antibody are also provided in the Table 1 as determined by the Kabat numbering. See Kabat, E. A., et al. (1991) Sequences of Proteins of Immunological Interest, Fifth Edition, U.S. Department of Health and Human Services, NIH Publication No. 91-3242. See also www2.mrc-lmb.cam.ac.uk/vbase/alignments2.php). Each of these exemplary anti-LILRB2 antibodies, as well as their functional equivalents ad disclosed herein, is within the scope of the present disclosure.

TABLE 1
Exemplary Anti-LILRB2 Antibodies

| | | | SEQ |
|---|---|---|---|
| Antibody | | SEQUENCE | ID NO |
| Antibody | VH CDR1 | GNSFTDNNIY | 1 |
| 10B11 | VH CDR2 | YIDPFNGRTTYNQKFKG | 2 |
| | VH CDR3 | GDYFNY | 3 |
| | VH | EIQLQQSGPELVKPGASVKVSCKASGNSFTDNNIYWVKQSHGESLEWIGYIDP | 4 |
| | | FNGRTTYNQKFKGKATLTVDKSSSTAFMHLNSLTSGDSAVYYCARGDYFNYWG | |
| | | QGTTLTVSS | |
| | VL CDR1 | SASQGINNYLN | 5 |
| | VL CDR2 | YTSSLHS | 6 |
| | VL CDR3 | QQYSNLPFT | 7 |
| | VL | EIQMTQTTSSLSASLGDRVTISCSASQGINNYLNWFQQKSDGTVKLLIYYTSS | 8 |
| | | LHSGVPSRFSGSGSGTDYSLTISNLEPEDIAIYYCQQYSNLPFTFGGGTELEI | |
| | | K | |
| Antibody | VH CDR1 | GYSITSGYYWN | 9 |
| 11B12 | VH CDR2 | YISYDGNNNYNPSLKN | 10 |
| | VH CDR3 | EESTMITTAWFAY | 11 |
| | VH | DVQLQESGPGLVKPSQSLSLTCSVTGYSITSGYYWNWIRQFPGNKLEWMGYIS | 12 |
| | | YDGNNNYNPSLKNRISITRDTSKNQFFLKLNSVTTEDTATYYCAREESTMITT | |
| | | AWFAYWGQGTLVTVSA | |
| | VL CDR1 | RASENIYSNLA | 13 |
| | VL CDR2 | GATNLAD | 415 |
| | VL CDR3 | QHFWDTPYT | 15 |
| | VL | DIQMTQSPASLSVSVGETVTITCRASENIYSNLAWYQQKQGKSPQLLVYGAIN | 16 |
| | | LADGVPSRFSGSGSGTQYSLKINSLQSEDFGSYFCQHFWDTPYTFGGGTKLEI | |
| | | K | |
| Antibody | VH CDR1 | GYAFTTYNMY | 17 |
| 11D9 | VH CDR2 | YIDPYNGGATYNLKFKG | 18 |
| | VH CDR3 | GDYFNY | 3 |
| | VH | EIQLQQSGPELVKPGASVKLSCKASGYAFTTYNMYWVKQSPGKSLEWIGYIDP | 19 |
| | | YNGGATYNLKFKGKATLTVDKSSSAAYMHLNGLTSEDSAVYYCAGGDYFNYWG | |
| | | QGTILTVSS | |
| | VL CDR1 | RASQVINNYLN | 20 |
| | VL CDR2 | YTSTLHS | 21 |
| | VL CDR3 | QQYSTLPYT | 22 |
| | VL | DIQMTQTTSSLSASLGDRVTISCRASQVINNYLNWFQQKPDGTVKLLIYYTST | 23 |
| | | LHSGVPSRFSGSGSGTDYSLTISNLEPEDFATYYCQQYSTLPYTFGGGTKLEI | |
| | | K | |
| Antibody | VH CDR1 | GSSFTDYNMY | 24 |
| 4A9 | VH CDR2 | YIDPYNGRTTYNQKFKG | 25 |
| | VH CDR3 | GDYFDY | 26 |
| | VH | EIQLQQSGPELVKPGASVKVSCKASGSSFTDYNMYWVKQIHGKSLDWIGYIDP | 27 |
| | | YNGRTTYNQKFKGKATLTVDKSSSTAFMHLNSLTSEDSAVYYCAGGDYFDYWG | |
| | | HGTSLTVSS | |
| | VL CDR1 | SASQGISNYLN | 28 |
| | VL CDR2 | YTSTLHS | 21 |
| | VL CDR3 | QQYSNLPYT | 29 |
| | VL | EIQMTQTTSSLSASLGDRVTISCSASQGISNYLNWFQQKPDGTVKLLIYYTST | 30 |
| | | LHSGVPSRFSGSGSGTDFSLTISNLDPEDFATYYCQQYSNLPYTFGGGTKLEI | |
| | | K | |
| Antibody | VH CDR1 | GFTFSSYGMS | 31 |
| 10D7 | VH CDR2 | TISGAFSYTYYPDSVKG | 32 |
| | VH CDR3 | HDNSLYYAMDY | 33 |
| | VH | EVKLVESGGGLVKPGGSLKLSCAASGFTFSSYGMSWVRQTPEKRLEWVATISG | 34 |
| | | AFSYTYYPDSVKGRFTISRDNAKNNLYLQMSSLRSEDTAFYYCARHDNSLYYA | |
| | | MDYWGQGTSVTVSS | |
| | VL CDR1 | RTSKSITKYLA | 35 |
| | VL CDR2 | SGSTLQS | 36 |
| | VL CDR3 | QQHNEYPWT | 37 |
| | VL | NVQITQSPSYLAASPGETITINCRISKSITKYLAWYQEQPGKINKLLIYSGST | 38 |
| | | LQSGIPSRFSGSGSGTDFTLTISSLEPEDFAMYYCQQHNEYPWTFGGGTKLEI | |
| | | K | |
| Antibody | VH CDR1 | GSSFTDYNMY | 24 |
| 13H5 | VH CDR2 | YIDPYNGRTTYNQKFRG | 39 |
| | VH CDR3 | GDYFDY | 26 |
| | VH | EIQLQQSGPELVKPGTSVKVSCKASGSSFTDYNMYWVKQSHGKSLDWIGYIDP | 40 |
| | | YNGRTTYNQKFRGKATLTVDKSSSTAFMHLNSLTSEDSAVYYCARGDYFDYWG | |
| | | QGTSLTVSS | |
| | VL CDR1 | SASQGISNYLN | 28 |
| | VL CDR2 | YTSTLHS | 21 |
| | VL CDR3 | QQYSNLPYT | 29 |
| | VL | EIQMTQTTSSLSASLGDRVTISCSASQGISNYLNWFQQKPDGTVKLLIYYTST | 30 |
| | | LHSGVPSRFSGSGSGTDFSLTISNLDPEDFATYYCQQYSNLPYTFGGGTKLEI | |
| | | K | |
| Antibody | VH CDR1 | GFTFSTYGMS | 41 |
| 15A6 | VH CDR2 | TISGSNSYTYYPDSVKG | 42 |
| | VH CDR3 | HENGYYFYAMDY | 43 |
| | VH | EVKLVESGGDLVKPGGSLKLSCAASGFTFSTYGMSWVRQTPEKRLEWVATISG | 44 |
| | | SNSYTYYPDSVKGRFTISRDNAKNNLYLQMSSLRSEDTALYYCTRHENGYYFY | |
| | | AMDYWGQGTSVTVSS | |
| | VL CDR1 | RSSKSITKYLA | 45 |
| | VL CDR2 | SGSTLQS | 36 |
| | VL CDR3 | QQHSEYPWT | 46 |
| | VL | DVQITQSPSYLTASPGETITINCRSSKSITKYLAWYQEKPGKINKLLIYSGST | 47 |
| | | LQSGIPSRFSGSGSGTDFTLTISSLEPEDFAMYYCQQHSEYPWTFGGGTRLEI | |
| | | K | |
| Antibody | VH CDR1 | GYTFTNYGMN | 48 |
| 6E8 | VH CDR2 | WMNTYIGEPTYADDFKG | 49 |
| | VH CDR3 | RGDYASYYFDY | 50 |
| | VH | QIHLVQSGPELKKPGETVKISCKASGYTFTNYGMNWVKQVPGKALKWMGWMNT | 51 |
| | | YIGEPTYADDFKGRFAFSLETSASTAYLQINNLKNEDMATYFCARRGDYASYY | |
| | | FDYWGQGTTLTVSS | |
| | VL CDR1 | RASQSISNNLH | 52 |
| | VL CDR2 | YASQSIS | 53 |
| | VL CDR3 | QQSNSWPHT | 54 |
| | VL | DIVLTQSPATLSVTPGDSVSLSCRASQSISNNLHWYHQKSHESPRLLIKYASQ | 55 |
| | | SISGIPSRVSGSGSGTDFTLSIITVETEDFGMYFCQQSNSWPHTFGGGTKLEI | |
| | | R | |
| Antibody | VH CDR1 | GYTFTNYGMN | 48 |
| 6H8 | VH CDR2 | WINTYTGEPTYADDFRG | 56 |
| | VH CDR3 | YYDYDYFD | 57 |
| | VH | QIQLVQSGPELKKPGETVRISCKASGYTFTNYGMNWVKQAPGKGLKWMGWINT | 58 |
| | | YTGEPTYADDERGRFALSLEPSASTASLQINNLKNEDTATYFCARYYDYDYED | |
| | | YWGQGTTLTVSS | |
| | VL CDR1 | KSSQSLLNGGNQKNYLA | 59 |
| | VL CDR2 | GASTRVS | 60 |
| | VL CDR3 | QNDLSYPLT | 61 |
| | VL | DIVMTQSPSSLRVSAGEKVTMSCKSSQSLLNGGNQKNYLAWYQQKPGQPPKVL | 62 |
| | | IYGASTRVSGVPDRFTGRGSGIDFTLTISSVEAEDLAVYYCQNDLSYPLTFGA | |
| | | GTKLELK | |
| Antibody | VH CDR1 | GYTFTNYGMN | 48 |
| 13C4 | VH CDR2 | WMNTYIGEPTYADDFKG | 49 |
| | VH CDR3 | RGDYASYYFDY | 50 |
| | VH | QIHLVQSGPELKKPGETVKISCKASGYTFTNYGMNWVKQAPGKALKWMGWMNT | 63 |
| | | YIGEPTYADDFKGRFAFSLETSASTAYLQINNLKNEDMATYFCARRGDYASYY | |
| | | FDYWGQGTTLTVSS | |
| | VL CDR1 | RASQNINNNLH | 64 |
| | VL CDR2 | YASQSIS | 53 |
| | VL CDR3 | QQSNSWPHT | 54 |
| | VL | DIVLTQSPATLSVTPGDSVSLSCRASQNINNNLHWYHQKSHESPRLLIKYASQ | 65 |
| | | SISGIPSRVSGSGSGTDFTLSIVTVETEDFGMFFCQQSNSWPHTFGGGTKLEI | |
| | | R | |
| Antibody | VH CDR1 | GYAFTSHNIY | 66 |
| 13D12 | VH CDR2 | YIDPYNGGSTYNQKFKD | 67 |
| | VH CDR3 | GDYRDF | 68 |
| | VH | EIQLQQSGPELVKPGASVKVSCKASGYAFTSHNIYWVKQSHGKSLEWIGYIDP | 69 |
| | | YNGGSTYNQKFKDKATLTVDKSSSTAYMHLKSLTSEDSAVYYCARGDYRDFWG | |
| | | QGTTLTVSS | |
| | VL CDR1 | SASQAINNYLN | 70 |
| | VL CDR2 | YTSSLHS | 6 |
| | VL CDR3 | QQYSHLPWT | 71 |
| | VL | DIQMTQTTSSLSASLGDRVTIRCSASQAINNYLNWYQQKPDGTVKLLIYYTSS | 72 |
| | | LHSGVPSRFSGSGSGADYSLTISNLEPEDIATYYCQQYSHLPWTFGGGTTLEI | |
| | | K | |
| Antibody | VH CDR1 | GFTFSNFGMH | 73 |
| 9B6 | VH CDR2 | YISSGSGTIYYSDTVRG | 74 |
| | VH CDR3 | SDYDGDWFFDV | 75 |
| | VH | DVQLVESGGGLVQPGWSRILSCAASGFTFSNFGMHWVRQAPEKGLEWVAYISS | 76 |
| | | GSGTIYYSDTVRGRFTISRDNPKNTLFLLMTSLRSEDTAMYYCTRSDYDGDWF | |
| | | FDVWGAGTTVTVSS | |
| | VL CDR1 | KASQDVTTAVA | 77 |
| | VL CDR2 | SASFRYT | 78 |
| | VL CDR3 | QQHFTTPLT | 79 |
| | VL | DIVMTQSHRFLSTSVGDRVTITCKASQDVTTAVAWCQQKPGQSPKLLIYSASF | 80 |
| | | RYTGVPDRFTGSGSGTDFTFTINNVQAEDLALYYCQQHFTTPLTFGAGTKLEL | |
| | | K | |
| Antibody | VH CDR1 | GFTFSD-FYMG | 81 |
| 2C1 | VH CDR2 | ASGNKAHDYATEYSASVKG | 82 |
| | VH CDR3 | SYYYGSSAWFTY | 83 |
| | VH | EVKLVEPGGGLVQPGGSLRLSCATSGFTFSDFYMGWVRQPPGKRLEWIAASGN | 84 |
| | | KAHDYATEYSASVKGRFFVSRDTFQSILYLQMNALRAEDTAIYYCARSYYYGS | |
| | | SAWFTYWGQGTLVTVSV | |
| | VL CDR1 | KASQDVGTAVA | 85 |
| | VL CDR2 | WASTRHA | 86 |
| | VL CDR3 | QQYSSYPLT | 87 |
| | VL | DIVMTQSHKFMSTSIRDRVTIACKASQDVGTAVAWYQQKPGQSPKLLIFWAST | 88 |
| | | RHAGVPDRFTGSGSGTDFTLTISNVQSEDLTDYFCQQYSSYPLTFGAGTKLEL | |
| | | K | |
| Antibody | VH CDR1 | GFSLTSYGVH | 89 |
| 3C12 | VH CDR2 | VIWAGGTTNYNSALMS | 90 |
| | VH CDR3 | GGYGHSMDY | 91 |
| | VH | QVQLKESGPGLVAPSQSLSITCTVSGFSLTSYGVHWVRQSPGKGLEWLGVIWA | 92 |
| | | GGTTNYNSALMSRLSISKDNSKSQVFLKMNSLQADDTAMYYCARGGYGHSMDY | |
| | | WGQGTSVTVSS | |
| | VL CDR1 | KASQDVSNAVA | 93 |
| | VL CDR2 | SASYRYT | 94 |
| | VL CDR3 | QQHYSTPFT | 95 |
| | VL | DIVMTQSHKFMSTSVGDRVSITCKASQDVSNAVAWYQQKPGQSPKLLIYSASY | 96 |
| | | RYTGVPDRFTGSGSGTDFIFTISSVQAEDLAVYYCQQHYSTPFTFGSGTKLEI | |
| | | K | |
| Antibody | VH CDR1 | GYIFTDFNMH | 97 |
| 7C12 | VH CDR2 | YIYPFNGGTGYNQKFEN | 98 |
| | VH CDR3 | RGGRGHFDY | 99 |
| | VH | EVQLQQSGPDLVKPGASVKISCKASGYIFTDFNMHWVKQSHGKSLEWIGYIYP | 100 |
| | | FNGGTGYNQKFENMATLTVDISSSTAYMEVRSLTSEDSAVYYCARRGGRGHED | |
| | | YWGQGTTLTVSS | |
| | VL CDR1 | RASGNIHNYLA | 101 |
| | VL CDR2 | NAKTLAD | 102 |
| | VL CDR3 | QHFWNTPFT | 103 |
| | VL | DIQMTQSPASLSASVGETVTVTCRASGNIHNYLAWYQQKQGKSPQLLVYNAKT | 104 |
| | | LADGVPSRFSGSGSGIQFSLKINSLQPEDFGSYYCQHFWNTPFTFGSGTKLEI | |
| | | K | |
| Antibody | VH CDR1 | GFSLATYGVH | 105 |
| 7G4 | VH CDR2 | VMWRDGSTDYNAVEMS | 106 |
| | VH CDR3 | NPYLYGSSHYYAMDY | 107 |
| | VH | QVQLKQSGPGLVQPSQSLSITCTVSGFSLATYGVHWVRQSPGKGLEWLGVMWR | 108 |
| | | DGSTDYNAVEMSRLSITKDDSKSQVFFKMNSLQADDTAIYYCAKNPYLYGSSH | |
| | | YYAMDYWGQGTSVTVSS | |
| | VL CDR1 | KSSQSLLNSENQKNYLA | 109 |
| | VL CDR2 | GASTRES | 110 |
| | VL CDR3 | QNDHIYPFT | 111 |
| | VL | GDIVMTQSPSSLSVSAGEKVTMSCKSSQSLLNSENQKNYLAWYQQKPGQPPKL | 112 |
| | | LIYGASTRESGVPDRFTGSGSGTDFTLTISSVQAEDLAVYYCQNDHIYPFTFG | |
| | | SGTKLEIK | |
| Antibody | VH CDR1 | GFSLTDYGVN | 113 |
| 8A4 | VH CDR2 | MIWGDGNTDYNSALKS | 114 |
| | VH CDR3 | SSYRYDENIYYYAMDY | 115 |
| | VH | QVQLKESGPGLVAPSQSLSITCTVSGFSLTDYGVNWVRQPPGKGLEWLGMIWG | 116 |
| | | DGNTDYNSALKSRLSISKVNSKSQVFLKMNSLQTDDTARYYCARSSYRYDENI | |
| | | YYYAMDYWGQGTSVTVSS | |
| | VL CDR1 | KSSQSLLNSGIQKNYLT | 117 |
| | VL CDR2 | WASTRES | 118 |
| | VL CDR3 | QNDYSLPFT | 119 |
| | VL | DIVMTQSPSSLTVTAGEEVTLSCKSSQSLLNSGIQKNYLTWYQQKPGQPPKLL | 120 |
| | | IYWASTRESGVPDRFTGSVSGTDFTLTISSVQAEDLAVYYCQNDYSLPFTFGS | |
| | | GTKLEIK | |
| Antibody | VH CDR1 | GFNVKDYYMH | 121 |
| 9H6 | VH CDR2 | WIDPKNGNGIYDPKFQG | 122 |
| | VH CDR3 | GDYFGSSYLFAY | 123 |
| | VH | EVQLQQSGAELVRPGALVKLSCKASGFNVKDYYMHWVKQRPEQGLEWIGWIDP | 124 |
| | | KNGNGIYDPKFQGKASITADTSSNTAYLQLSSLTSEDTSVYYCARGDYFGSSY | |
| | | LFAYWGQGTLVTVSA | |
| | VL CDR1 | KASQDVSTAVA | 125 |
| | VL CDR2 | SASYRYT | 94 |
| | VL CDR3 | QQHYSTPWT | 126 |
| | VL | DIVMTQSHKFMSTSVGDRVSITCKASQDVSTAVAWYQQKPGQSPKLLIYSASY | 127 |
| | | RYTGVPDRFTGSGSGTDFTFTISSVQAEDLAVYYCQQHYSTPWTFGGGAKLEI | |
| | | K | |
| Antibody | VH CDR1 | GFSLTSYGVH | 89 |
| 12D8 | VH CDR2 | VIWSGGITDYNAAFIS | 128 |
| | VH CDR3 | EIYGNPDY | 129 |
| | VH | QVQLRQSGPGLVQPSQSLSITCTVSGFSLTSYGVHWVRQSPGKGLEWLGVIWS | 130 |
| | | GGITDYNAAFISRLSISKDNSKSQVFFRMNSLQTNDTAIYYCAREIYGNPDYW | |
| | | GQGTTLTVSS | |
| | VL CDR1 | KASQDVSIAVA | 131 |
| | VL CDR2 | SASYRYT | 94 |
| | VL CDR3 | QQHYSTPRT | 132 |
| | VL | DIVMTQSHKFMSTSVGDRVSITCKASQDVSIAVAWFQQKPGQSPKLLIYSASY | 133 |
| | | RYTGVPDRFSGSGSGTDFTFTISSVQAEDLAVYYCQQHYSTPRTFGGGTKLEI | |
| | | K | |
| Antibody | VH CDR1 | GFSLTTFGVH | 134 |
| 13C5 | VH CDR2 | VIWRGGSTDYNAAFMS | 135 |
| | VH CDR3 | NPYQYGSSHYYAMDY | 136 |
| | VH | QVQLKQSGPGLVQPSQSLSITCTVSGFSLTTFGVHWVRQSPGKGLEWLGVIWR | 137 |
| | | GGSTDYNAAFMSRLSITKDNSKSQVFFKMNSLQADDTAIYYCAKNPYQYGSSH | |
| | | YYAMDYWGQGTSVTVSS | |
| | VL CDR1 | KSSQSLLNSGNQKNYLA | 138 |
| | VL CDR2 | GASTRES | 110 |
| | VL CDR3 | QNDHILPFT | 139 |
| | VL | DIVMTQSPSSLSVSAGEKVTMSCKSSQSLLNSGNQKNYLAWYQQKPGQPPKLL | 140 |
| | | IYGASTRESGVPDRFTGSGSGTVFTLTISSVQAEDLAVYYCQNDHILPFTFGS | |
| | | GTKLEIK | |
| Antibody | VH CDR1 | GFSLTTYGVH | 141 |
| 14F1 | VH CDR2 | VIWTGGSTNYNSALMS | 142 |
| | VH CDR3 | GGYGHTMDY | 143 |
| | VH | QVQLKESGPGLVAPSQSLSITCTVSGFSLTTYGVHWVRQPPGKGLEWLGVIWT | 144 |
| | | GGSTNYNSALMSRLSIIKDNSKSQVFLKMNSLQTDDTAIYYCARGGYGHTMDY | |
| | | WGQGTSVTVSS | |
| | VL CDR1 | KASQDVSIAVA | 131 |
| | VL CDR2 | SASYRYT | 94 |
| | VL CDR3 | QQHYSTPFT | 95 |
| | VL | DIVMTQSHKFMSTSVGDRVSITCKASQDVSIAVAWYQQKPGQFPKLLIYSASY | 145 |
| | | RYTGVPDRFTGGGSGTDFTFTISSVQAEDLAVYYCQQHYSTPFTFGSGTKLEI | |
| | | K | |
| Antibody | VH CDR1 | GFSLTSYGVH | 89 |
| 15E2 | VH CDR2 | VIWSGGITDYNAAFIS | 128 |
| | VH CDR3 | EIYGNPDY | 129 |
| | VH | QVQLRQSGPGLVQPSQSLSITCTVSGFSLTSYGVHWVRQSPGKGLEWLGVIWS | 130 |
| | | GGITDYNAAFISRLSISKDNSKSQVFFRMNSLQTNDTAIYYCAREIYGNPDYW | |
| | | GQGTTLTVSS | |
| | VL CDR1 | KASQDVSIAVA | 131 |
| | VL CDR2 | SASYRYT | 94 |
| | VL CDR3 | QQHYSTPRT | 132 |
| | VL | DIVMTQSHKFMSTSVGDRVSITCKASQDVSIAVAWFQQKPGQSPKLLIYSASY | 133 |
| | | RYTGVPDRFSGSGSGTDFTFTISSVQAEDLAVYYCQQHYSTPRTFGGGTKLEI | |
| | | K | |
| Antibody | VH CDR1 | GFTFSDAWMD | 146 |
| 2F1 | VH CDR2 | EIRSKSNNHTTYYAESVKG | 147 |
| | VH CDR3 | DWLTY | 148 |
| | VH | EVKLEESGGGLVQPGGSMKLSCAASGFTFSDAWMDWVRQSPKKGLEWVAEIRS | 149 |
| | | KSNNHTTYYAESVKGRFTISRDDSKSSVYLQMNSLRAEDTGIYYCTRDWLTYW | |
| | | GQGTLVTVSA | |
| | VL CDR1 | RASKSVSTSGYSYMN | 150 |
| | VL CDR2 | LASNLES | 151 |
| | VL CDR3 | QHSRELPLT | 152 |
| | VL | DIVLTQSPASLAVSLGQRATISCRASKSVSTSGYSYMNWYQQKPGQPPKLLIY | 153 |
| | | LASNLESGVPARFSGSGSGTDFTLNIHPVEEEDAATYYCQHSRELPLTFGAGT | |
| | | KLELK | |
| Antibody | VH CDR1 | GYTFTDYNMH | 154 |
| 3B5 | VH CDR2 | SIYPYNGDTDYNQKFKS | 155 |
| | VH CDR3 | ENDYDGPPFPY | 156 |
| | VH | EVQLQQSGPELVKPGASLKISCKASGYTFTDYNMHWVKQSHGKSLEWIGSIYP | 157 |
| | | YNGDTDYNQKFKSKATLTIDNSSSTAFMDLRSLTSEDSAVYYCARENDYDGPP | |
| | | FPYWGLGTLVTVSA | |
| | VL CDR1 | KASEDIYNRLA | 158 |
| | VL CDR2 | GATSLET | 159 |
| | VL CDR3 | QQYWSIRT | 160 |
| | VL | DIQMTQSSSSFSVSLGDRVTITCKASEDIYNRLAWYQQKSGNAPRLLISGATS | 161 |
| | | LETGVPSRFSGSGSGKDYTLSITSLQTEDVATYYCQQYWSIRTFGGGTKLEIK | |
| Antibody | VH CDR1 | GYIFTDFNMH | 97 |
| 14C11 | VH CDR2 | YIYPFNGGTGYNQKFEN | 98 |
| | VH CDR3 | RGGRGHFDY | 99 |
| | VH | EVQLQQSGPDLVKPGASVKISCKASGYIFTDFNMHWVKQSHGKSLEWIGYIYP | 162 |
| | | FNGGTGYNQKFENKATLTVDNSSSTAYMEVRSLTSEDSAVYYCARRGGRGHED | |
| | | YWGQGTTLTVSS | |
| | VL CDR1 | RASGNIHNYLA | 101 |
| | VL CDR2 | NAKTLAD | 102 |
| | VL CDR3 | QHFWNTPFT | 103 |
| | VL | DIQMTQSPASLSASVGETVTVTCRASGNIHNYLAWYQQKQGKSPQLLVYNAKT | 163 |
| | | LADGVPSRFSGSGSGTQFSLKINSLQPEDFGSYYCQHFWNTPFTFGSGTKLEI | |
| | | K | |
| Antibody | VH CDR1 | GYTFTSYWMH | 164 |
| SLL-1 | VH CDR2 | EINPTNGRINYNENFKS | 165 |
| | VH CDR3 | DTTATFAY | 166 |
| | VH | QVQLQQPGAELVKPGASVKLSCKASGYTFTSYWMHWVKQRPGQGLEWIGEINP | 167 |
| | | TNGRTNYNENFKSKATLTVDISSSTAYMQLSSLTSEDSAVYYCARDTTATFAY | |
| | | WGQGTLVTVSA | |
| | VL CDR1 | RASDNINSNLA | 168 |
| | VL CDR2 | AAANLAD | 169 |
| | VL CDR3 | QHFWGTPYT | 170 |
| | VL | DIQMTQSPASLSVSVGETVIITCRASDNINSNLAWYQQKQGKSPQLLVYAAAN | 171 |
| | | LADGVPSRFSGSGSGTQYSLKINSLQSEDFGTYYCQHFWGTPYTFGGGTKLEI | |
| | | K | |
| Antibody | VH CDR1 | GYSFTDSNMY | 172 |
| SLL-2 | VH CDR2 | YIDPYNGGSRYNQKFKG | 173 |
| | VH CDR3 | GDYFDY | 26 |
| | VH | EIQLQQSGPELVKPGASVKVSCKASGYSFTDSNMYWVKQSHGKSLEWIGYIDP | 174 |
| | | YNGGSRYNQKFKGKATLTVDKSSSTAFMHLNSLTSGDSAVYYCARGDYFDYWG | |
| | | RGTTLTVSS | |
| | VL CDR1 | SASQGISNYLN | 28 |
| | VL CDR2 | YTSSLYS | 175 |
| | VL CDR3 | QQYSNLPYT | 29 |
| | VL | DIQMTQTTSSLSASLGDRVTISCSASQGISNYLNWYQQKPDGTVKLLIYYTSS | 176 |
| | | LYSGVSSRFSGSGSGTDYSLIISNLEPEDIATYYCQQYSNLPYTFGGGTKLEI | |
| | | K | |
| Antibody | VH CDR1 | GFTFSDFYME | 177 |
| SLL-3 | VH CDR2 | ASRNKANDYTTDYSASVKG | 178 |
| | VH CDR3 | VYYRYWYFDV | 179 |
| | VH | EVKLVESGGGLVQPGGSLRLSCATSGFTFSDFYMEWVRQPPGERLEWIAASRN | 180 |
| | | KANDYTTDYSASVKGRFIVSRDTSQSILYLQMNALRAEDTAIYYCARVYYRYW | |
| | | YFDVWGAGTTVTVSS | |
| | VL CDR1 | KASQDVSFAVA | 181 |
| | VL CDR2 | WASTRHT | 182 |
| | VL CDR3 | QQHYSAPPT | 183 |
| | VL | DIVMTQSHKFMSTFVGDRVSITCKASQDVSFAVAWYQRKPGQSPKLLIYWAST | 184 |
| | | RHTGVPDRFTGSGSGTDYTLTISSVQAEDLALYYCQQHYSAPPTFGGGTKLEI | |
| | | K | |
| Antibody | VH CDR1 | GYSFTDSNMY | 172 |
| SLL-4 | VH CDR2 | YIDPYNGGTRYNQKFKG | 185 |
| | VH CDR3 | GDYFDC | 186 |
| | VH | EIQLQQSGPELVKPGASVKVSCKASGYSFTDSNMYWVKQSHGKSLEWIGYIDP | 187 |
| | | YNGGTRYNQKFKGKATLTVDKSSSTAFMHLNSLTSEDSAVYYCARGDYFDCWG | |
| | | QGTTLTVSS | |
| | VL CDR1 | SASQGISNYLN | 28 |
| | VL CDR2 | YTSSLYS | 175 |
| | VL CDR3 | QQYSNLPYT | 29 |
| | VL | DIQMTQTTSSLSASLGDRVTISCSASQGISNYLNWYQQKPDGTVKLLIYYTSS | 188 |
| | | LYSGVSSRFSGSGSGTDYSLTISNLEPEDVATYYCQQYSNLPYTFGGGTKLEI | |
| | | K | |
| Antibody | VH CDR1 | GYTFTSYRMH | 189 |
| SLL-5 | VH CDR2 | EINPINGRTNYNENFKS | 165 |
| | VH CDR3 | DTTATFAY | 166 |
| | VH | QVQLQQPGAELVKPGASVKLSCKASGYTFTSYRMHWVKQRPGQGLEWIGEINP | 190 |
| | | TNGRTNYNENFKSKATLTVDKSSSTAYMQLSSLTSEDSAVYYCARDTTATFAY | |
| | | WGQGTLVTVSA | |
| | VL CDR1 | RASDNINSNLA | 168 |
| | VL CDR2 | AAANLAD | 169 |
| | VL CDR3 | QHFWGTPYT | 170 |
| | VL | DIQMTQSPASLSVSVGETVIITCRASDNINSNLAWYQQKQGKSPQLLVYAAAN | 171 |
| | | LADGVPSRFSGSGSGTQYSLKINSLQSEDFGTYYCQHFWGTPYTFGGGTKLEI | |
| | | K | |

In some embodiments, the anti-LILRB2 antibodies described herein bind to the same epitope of a LILRB2 polypeptide as any of the exemplary (reference) antibodies described herein or compete against the exemplary antibody from binding to the LILRB2 antigen. An “epitope” refers to the site on a target antigen that is recognized and bound by an antibody. The site can be entirely composed of amino acid components, entirely composed of chemical modifications of amino acids of the protein (e.g., glycosyl moieties), or composed of combinations thereof. Overlapping epitopes include at least one common amino acid residue. An epitope can be linear, which is typically 6-15 amino acids in length. Alternatively, the epitope can be conformational. The epitope to which an antibody binds can be determined by routine technology, for example, the epitope mapping method (see, e.g., descriptions below). An antibody that binds the same epitope as an exemplary antibody described herein may bind to exactly the same epitope or a substantially overlapping epitope (e.g., containing less than 3 non-overlapping amino acid residues, less than 2 non-overlapping amino acid residues, or only 1 non-overlapping amino acid residue) as the exemplary antibody. Whether two antibodies compete against each other from binding to the cognate antigen can be determined by a competition assay, which is well known in the art.

In some examples, the anti-LILRB2 antibody comprises the same V_{H} and/or V_{L} CDRs as an exemplary antibody described herein. Two antibodies having the same V_{H} and/or V_{L} CDRs means that their CDRs are identical when determined by the same approach (e.g., the Kabat approach, the Chothia approach, the AbM approach, the Contact approach, or the JMGT approach as known in the art. See, e.g., bioinf.org.uk/abs/). Such anti-LILRB2 antibodies may have the same V_{H}i, the same V_{L}, or both as compared to an exemplary antibody described herein.

Exemplary Humanized Antibodies

In some embodiments, the anti-LILRB2 antibody disclosed herein can be a humanized antibody as disclosed herein, which may be derived from any of the exemplary antibodies provided in Table 1 above.

In some instances, the humanized antibody may be derived from the parent antibody 11B12. Such a humanized antibody may comprise the same heavy chain and light chain CDRs as those of antibody 11B12 (see Table 1 above) and the heavy chain and light chain framework regions (HC FR1-F4 and LC FR1-FR4) provided in Table 2 below.

TABLE 2
Humanized and Affinity Maturation Variants of Clone 11B12

| Antibody | | | |
|---|---|---|---|
| Elements | | SEQUENCES | SEQ ID NO |
| HC FR1 Motif | | EVQLVESGGGLVQPGGSLRLSCAZ₁S, in which Z₁ is A or V | 191 |
| HC CDR1 Motif | | GX₁SITSGYX₂WX₃, in which X₁ is Y or G, X₂ is Y, S, A, | 192 |
| | | or H, and X₃ is N, S, or W | |
| HC FR2 Motif | | WZ₂RQAPGKGLEWVA, in which Z₂ is I or V | 193 |
| HC CDR2 Motif | | X₄ISYDGNX₅X₆, in which X₄ is S, T, or Y, X₅ is A, I, | 194 |
| | | K, L, M, N, P, S, I, V, or W and X₆ is H, L, N, S, or | |
| | | W | |
| | | Optionally X₄ISYDGNX₅X₆YNPSLKN | 416 |
| HC FR3 Motif | | RFTISRDZ₃SKNTLZ₄LQMNSLRAEDTAVYYCZ₅R, in which Z₃ is A, | 195 |
| | | D or T, Z₄ is F, L, or V, and Z₅ is A or V | |
| HC CDR3 Motif | | EEX₇TMX₈TTX₉WFX₁₀Y, in which X₇ is A or S, X₈ is F or I, | 196 |
| | | X₉ is A or G, and X₁₀ is A, G, Q or S | |
| HC FR4 Motif | | WGQGTLVTVSS | 197 |
| LC FR1 Motif | | DIQZ₆TQSPSSLSASVGDRVTITC, in which Z₆ is L or M | 198 |
| LC CDR1 Motif | | X₁₁X₁₂SEX₁₃IX₁₄SNX₁₅A, in which X₁₁ is E, G, L, N, Q, R, | 199 |
| | | S, T or V, X₁₂ is A or G, X₁₃ is K, N, T, or V, X₁₄ is | |
| | | F or Y, and X₁₅ is L, N, or Q | |
| LC FR2 Motif | | WYQQKPGKAPKLLIY | 200 |
| LC CDR2 Motif | | GATX₁₆X₁₇X₁₈X₁₉, in which X₁₆ is E, N, W, or Y, X₁₇ is | 201 |
| | | F, H, L, P, or R, X₁₈ is A, N, R, or S, and X₁₉ is | |
| | | A, G, K, L, R, S, or V | |
| LC FR3 Motif | | GVPSRFSGSGSGTDZ₇TLTISSLQPEDFATYYC, in which Z₇ is F | 202 |
| | | or Y | |
| LC CDR3 Motif | | QX₂₀FWDX₂₁PYX₂₂, in which X₂₀ is H or Q, X₂₁ is L, S, | 203 |
| | | T, or Y, and X₂₂ is I, M, R, T, Y, or V | |
| LC FR4 Motif | | FGQGTKVEIK | 204 |
| Antibody | VH | EVQLVESGGGLVQPGGSLRLSCAASGYSITSGYYWNWVRQAPGKGLEWVAYI | VH CDR1-CDR3: |
| h#2a | | SYDGNNNYNPSLKNRFTISRDDSKNTLYLQMNSLRAEDTAVYYCAREESTMI | 9-11 |
| | | TTAWFAYWGQGTLVTVSS | VH: 205 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDFTLTISSLQPEDFATYYCQHFWDTPYTFGQGTKV | 13-15 |
| | | EIK | VL: 206 |
| Antibody | VH | EVQLVESGGGLVQPGGSLRLSCAVSGYSITSGYYWNWIRQAPGKGLEWVAYI | VH CDR1-CDR3: |
| h#2B1 | | SYDGNNNYNPSLKNRFTISRDASKNTVYLQMNSLRAEDTAVYYCAREESTMI | 9-11 |
| | | TTAWFAYWGQGTLVTVSS | VH: 207 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWVRQAPGKGLEWVAYI | VH CDR1-CDR3: |
| h#2C1 | | SYDGNNNYNPSLKNRFTISRDTSKNTFYLQMNSLRAEDTAVYYCAREESTMI | 9-11 |
| | | TTAWFAYWGQGTLVTVSS | VH: 209 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWVRQAPGKGLEWVAYI | VH CDR1-CDR3: |
| h#2D1 | | SYDGNNNYNPSLKNRFTISRDTSKNTVYLQMNSLRAEDTAVYYCVREESTMI | 9-11 |
| | | TTAWFAYWGQGTLVTVSS | VH: 210 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| h#2D2 | | SYDGNNNYNPSLKNRFTISRDASKNTFYLQMNSLRAEDTAVYYCAREESTMI | 9-11 |
| | | TTAWFAYWGQGTLVTVSS | VH: 211 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWVRQAPGKGLEWVAYI | VH CDR1-CDR3: |
| h#2D4 | | SYDGNNNYNPSLKNRFTISRDTSKNTFYLQMNSLRAEDTAVYYCVREESTMI | 9-11 |
| | | TTAWFAYWGQGTLVTVSS | VH: 212 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| h#2E1 | | SYDGNNNYNPSLKNRFTISRDTSKNTFYLQMNSLRAEDTAVYYCAREESTMI | 9-11 |
| | | TTAWFAYWGQGTLVTVSS | VH: 213 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| h#2E2 | | SYDGNNNYNPSLKNRFTISRDTSKNTVYLQMNSLRAEDTAVYYCAREESTMI | 9-11 |
| | | TTAWFAYWGQGTLVTVSS | VH: 214 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| h#2G3 | | SYDGNNNYNPSLKNRFTISRDTSKNTVYLQMNSLRAEDTAVYYCAREESTMI | 9-11 |
| | | TTAWFAYWGQGTLVTVSS | VH: 214 |
| | VL | DIQLTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 215 |
| Antibody | VH | EVQLVESGGGLVQPGGSLRLSCAVSGYSITSGYYWNWVRQAPGKGLEWVAYI | VH CDR1-CDR3: |
| h#2G6 | | SYDGNNNYNPSLKNRFTISRDASKNTFYLQMNSLRAEDTAVYYCAREESTMI | 9-11 |
| | | TTAWFAYWGQGTLVTVSS | VH: 216 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| 2E1_FC1 | | SYDGNVNYNPSLKNRFTISRDTSKNTFYLQMNSLRAEDTAVYYCAREESTMI | 9, 217, 11 |
| | | TTAWFAYWGQGTLVTVSS | VH: 218 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| 2E1_FG4 | | SYDGNVNYNPSLKNRFTISRDTSKNTFYLQMNSLRAEDTAVYYCAREESTMI | 9, 217, 11 |
| | | TTAWFAYWGQGTLVTVSS | VH: 218 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYIFGQGTKV | 13, 14, 219 |
| | | EIK | VL: 220 |
| Antibody | VH | EVQLVESGGGLVQPGGSLRLSCAVSGYSITSGYYWNWIRQAPGKGLEWVAYI | VH CDR1-CDR3: |
| 2E1_FG9 | | SYDGNVNYNPSLKNRFTISRDTSKNTFYLQMNSLRAEDTAVYYCAREESTMI | 9, 217, 11 |
| | | TTAWFAYWGQGTLVTVSS | VH: 218 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYRFGQGTKV | 13, 14, 221 |
| | | EIK | VL: 222 |
| Antibody | VH | EVQLVESGGGLVQPGGSLRLSCAVSGYSITSGYYWNWIRQAPGKGLEWVAYI | VH CDR1-CDR3: |
| 2E1_FE11 | | SYDGNVNYNPSLKNRFTISRDTSKNTFYLQMNSLRAEDTAVYYCAREESTMI | 9, 217, 11 |
| | | TTAWFAYWGQGTLVTVSS | VH: 218 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYYFGQGTKV | 13, 14, 223 |
| | | EIK | VL: 224 |
| Antibody | VH | EVQLVESGGGLVQPGGSLRLSCAVSGYSITSGYYWNWIRQAPGKGLEWVAYI | VH CDR1-CDR3: |
| 2E1_FE1 | | SYDGNMNYNPSLKNRFTISRDTSKNTFYLQMNSLRAEDTAVYYCAREESTMI | 9, 225, 11 |
| | | TTAWFAYWGQGTLVTVSS | VH: 226 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| 2E1_FD3 | | SYDGNANYNPSLKNRFTISRDTSKNTFYLQMNSLRAEDTAVYYCAREESTMI | 9, 227, 11 |
| | | TTAWFAYWGQGTLVTVSS | VH: 228 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| 2E1_FG2 | | SYDGNTNYNPSLKNRFTISRDTSKNTFYLQMNSLRAEDTAVYYCAREESTMI | 9, 229, 11 |
| | | TTAWFAYWGQGTLVTVSS | VH: 230 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYVFGQGTKV | 13, 14, 231 |
| | | EIK | VL: 232 |
| Antibody | VH | EVQLVESGGGLVQPGGSLRLSCAVSGYSITSGYYWNWIRQAPGKGLEWVAYI | VH CDR1-CDR3: |
| 2E1_FC3 | | SYDGNTNYNPSLKNRFTISRDTSKNTFYLQMNSLRAEDTAVYYCAREESTMI | 9, 229, 11 |
| | | TTAWFAYWGQGTLVTVSS | VH: 230 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVASI | VH CDR1-CDR3: |
| 2E1_FH2 | | SYDGNNNYNPSLKNRFTISRDTSKNTFYLQMNSLRAEDTAVYYCAREESTMI | 9, 233, 11 |
| | | TTAWFAYWGQGTLVTVSS | VH: 234 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| 2E1_FF3 | | SYDGNNSYNPSLKNRFTISRDTSKNTFYLQMNSLRAEDTAVYYCAREESTMI | 9, 235, 11 |
| | | TTAWFAYWGQGTLVTVSS | VH: 236 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| 2E1_FD4 | | SYDGNPNYNPSLKNRFTISRDTSKNTFYLQMNSLRAEDTAVYYCAREESTMI | 9, 237, 11 |
| | | TTAWFAYWGQGTLVTVSS | VH: 238 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VE | EVQLVESGGGLVQPGGSLRLSCAVSGYSITSGYYWNWIRQAPGKGLEWVAYI | VH CDR1-CDR3: |
| 2E1_FF4 | | SYDGNNWYNPSLKNRFTISRDTSKNTFYLQMNSLRAEDTAVYYCAREESTMI | 9, 239, 11 |
| | | TTAWFAYWGQGTLVTVSS | VH: 240 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| 2E1_FF5 | | SYDGNKNYNPSLKNRFTISRDTSKNTFYLQMNSLRAEDTAVYYCAREESTMI | 9, 241, 11 |
| | | TTAWFAYWGQGTLVTVSS | VH: 242 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| 2E1_FD6 | | SYDGNINYNPSLKNRFTISRDTSKNTFYLQMNSLRAEDTAVYYCAREESTMI | 9, 243, 11 |
| | | TTAWFAYWGQGTLVTVSS | VH: 244 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| 2E1_FF10 | | SYDGNINYNPSLKNRFTISRDTSKNTFYLQMNSLRAEDTAVYYCAREESTMI | 9, 243, 11 |
| | | TTAWFAYWGQGTLVTVSS | VH: 244 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYMFGQGTKV | 13, 14, 245 |
| | | EIK | VL: 246 |
| Antibody | VH | EVQLVESGGGLVQPGGSLRLSCAVSGYSITSGYYWNWIRQAPGKGLEWVAYI | VH CDR1-CDR3: |
| 2E1_FC11 | | SYDGNINYNPSLKNRFTISRDTSKNTFYLQMNSLRAEDTAVYYCAREESTMI | 9, 243, 11 |
| | | TTAWFAYWGQGTLVTVSS | VH: 244 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDYPYTFGQGTKV | 13, 14, 247 |
| | | EIK | VL: 248 |
| Antibody | VH | EVQLVESGGGLVQPGGSLRLSCAVSGYSITSGYYWNWIRQAPGKGLEWVAYI | VH CDR1-CDR3: |
| 2E1_FF9 | | SYDGNLNYNPSLKNRFTISRDTSKNTFYLQMNSLRAEDTAVYYCAREESTMI | 9, 249, 11 |
| | | TTAWFAYWGQGTLVTVSS | VH: 250 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| 2E1_FH9 | | SYDGNNLYNPSLKNRFTISRDTSKNTFYLQMNSLRAEDTAVYYCAREESTMI | 9, 251, 11 |
| | | TTAWFAYWGQGTLVTVSS | VH: 252 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| 2E1_FA1 | | SYDGNSNYNPSLKNRFTISRDTSKNTFYLQMNSLRAEDTAVYYCAREESTMI | 9, 253, 254 |
| | | TTAWFQYWGQGTLVTVSS | VH: 255 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| 2E1_FG8 | | SYDGNVNYNPSLKNRFTISRDTSKNTFYLQMNSLRAEDTAVYYCAREEATMI | 9, 217, 256 |
| | | TTAWFAYWGQGTLVTVSS | VH: 257 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| 2E1_FA11 | | SYDGNVNYNPSLKNRFTISRDTSKNTFYLQMNSLRAEDTAVYYCAREESTMI | 9, 217, 258 |
| | | TTAWFSYWGQGTLVTVSS | VH: 259 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| 2E1_FB11 | | SYDGNNHYNPSLKNRFTISRDTSKNTFYLQMNSLRAEDTAVYYCAREESTMF | 9, 260, 261 |
| | | TTAWFQYWGQGTLVTVSS | VH: 262 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| 2E1_GG3 | | SYDGNNNYNPSLKNRFTISRDTSKNTFYLQMNSLRAEDTAVYYCAREESTMI | 9-11 |
| | | TTAWFAYWGQGTLVTVSS | VH: 213 |
| | VL | DIQMTQSPSSLSASVGDRVTITCRASENIYSNLAWYQQKPGKAPKLLIYGAT | VL CDR1-CDR3: |
| | | NLAGGVPSRFSGSGSGTDYTLTISSLQPEDFATYYCQHFWDTPYTFGQGTKV | 13, 263, 15 |
| | | EIK | VL: 264 |
| Antibody | VH | EVQLVESGGGLVQPGGSLRLSCAVSGYSITSGYYWNWIRQAPGKGLEWVAYI | VH CDR1-CDR3: |
| 2E1_GH3 | | SYDGNNNYNPSLKNRFTISRDTSKNTFYLQMNSLRAEDTAVYYCAREESTMI | 9-11 |
| | | TTAWFAYWGQGTLVTVSS | VH: 213 |
| | VL | DIQMTQSPSSLSASVGDRVTITCRASENIYSNLAWYQQKPGKAPKLLIYGAT | VL CDR1-CDR3: |
| | | NLSSGVPSRFSGSGSGTDYTLTISSLQPEDFATYYCQHFWDTPYTFGQGTKV | 13, 265, 15 |
| | | EIK | VL: 266 |
| Antibody | VH | EVQLVESGGGLVQPGGSLRLSCAVSGYSITSGYYWNWIRQAPGKGLEWVAYI | VH CDR1-CDR3: |
| 2E1_GB5 | | SYDGNNNYNPSLKNRFTISRDTSKNTFYLQMNSLRAEDTAVYYCAREESTMI | 9-11 |
| | | TTAWFAYWGQGTLVTVSS | VH: 213 |
| | VL | DIQMTQSPSSLSASVGDRVTITCRASENIYSNLAWYQQKPGKAPKLLIYGAT | VL CDR1-CDR3: |
| | | NLNSGVPSRFSGSGSGTDYTLTISSLQPEDFATYYCQHFWDTPYTFGQGTKV | 13, 267, 15 |
| | | EIK | VL: 268 |
| Antibody | VH | EVQLVESGGGLVQPGGSLRLSCAVSGYSITSGYYWNWIRQAPGKGLEWVAYI | VH CDR1-CDR3: |
| 2E1_GC6 | | SYDGNNNYNPSLKNRFTISRDTSKNTFYLQMNSLRAEDTAVYYCAREESTMI | 9-11 |
| | | TTAWFAYWGQGTLVTVSS | VH: 213 |
| | VL | DIQMTQSPSSLSASVGDRVTITCRASENIYSNLAWYQQKPGKAPKLLIYGAT | VL CDR1-CDR3: |
| | | NHASGVPSRFSGSGSGTDYTLTISSLQPEDFATYYCQHFWDTPYTFGQGTKV | 13, 269, 15 |
| | | EIK | VL: 270 |
| Antibody | VH | EVQLVESGGGLVQPGGSLRLSCAVSGYSITSGYYWNWIRQAPGKGLEWVAYI | VH CDR1-CDR3: |
| 2E1_GH9 | | SYDGNNNYNPSLKNRFTISRDTSKNTFYLQMNSLRAEDTAVYYCAREESTMI | 9-11 |
| | | TTAWFAYWGQGTLVTVSS | VH: 213 |
| | VL | DIQMTQSPSSLSASVGDRVTITCRASENIYSNLAWYQQKPGKAPKLLIYGAT | VL CDR1-CDR3: |
| | | WLASGVPSRFSGSGSGTDYTLTISSLQPEDFATYYCQHFWDTPYTFGQGTKV | 13, 271, 15 |
| | | EIK | VL: 272 |
| Antibody | VH | EVQLVESGGGLVQPGGSLRLSCAVSGYSITSGYYWNWIRQAPGKGLEWVAYI | VH CDR1-CDR3: |
| 2E1_GB11 | | SYDGNNNYNPSLKNRFTISRDTSKNTFYLQMNSLRAEDTAVYYCAREESTMI | 9-11 |
| | | TTAWFAYWGQGTLVTVSS | VH: 213 |
| | VL | DIQMTQSPSSLSASVGDRVTITCRASENIYSNLAWYQQKPGKAPKLLIYGAT | VL CDR1-CDR3: |
| | | NLALGVPSRFSGSGSGTDYTLTISSLQPEDFATYYCQHFWDTPYTFGQGTKV | 13, 273, 15 |
| | | EIK | VL: 274 |
| Antibody | VH | EVQLVESGGGLVQPGGSLRLSCAVSGYSITSGYYWNWIRQAPGKGLEWVAYI | VH CDR1-CDR3: |
| 2E1_GC11 | | SYDGNNNYNPSLKNRFTISRDTSKNTFYLQMNSLRAEDTAVYYCAREESTMI | 9-11 |
| | | TTAWFAYWGQGTLVTVSS | VH: 213 |
| | VL | DIQMTQSPSSLSASVGDRVTITCRASENIYSNLAWYQQKPGKAPKLLIYGAT | VL CDR1-CDR3: |
| | | NLARGVPSRFSGSGSGTDYTLTISSLQPEDFATYYCQHFWDTPYTFGQGTKV | 13, 275, 15 |
| | | EIK | VL: 276 |
| Antibody | VH | EVQLVESGGGLVQPGGSLRLSCAVSGYSITSGYYWNWIRQAPGKGLEWVAYI | VH CDR1-CDR3: |
| 2E1_GF1 | | SYDGNNNYNPSLKNRFTISRDTSKNTFYLQMNSLRAEDTAVYYCAREESTMI | 9-11 |
| | | TTAWFAYWGQGTLVTVSS | VH: 213 |
| | VL | DIQMTQSPSSLSASVGDRVTITCRASEVIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 277, 14, 15 |
| | | EIK | VL: 278 |
| Antibody | VH | EVQLVESGGGLVQPGGSLRLSCAVSGYSITSGYYWNWIRQAPGKGLEWVAYI | VH CDR1-CDR3: |
| 2E1_GD2 | | SYDGNNNYNPSLKNRFTISRDTSKNTFYLQMNSLRAEDTAVYYCAREESTMI | 9-11 |
| | | TTAWFAYWGQGTLVTVSS | VH: 213 |
| | VL | DIQMTQSPSSLSASVGDRVTITCTASENIYSNLAWYQQKPGKAPKLLIYGAT | VL CDR1-CDR3: |
| | | NPASGVPSRFSGSGSGTDYTLTISSLQPEDFATYYCQHFWDTPYTFGQGTKV | 279, 280, 15 |
| | | EIK | VL: 281 |
| Antibody | VH | EVQLVESGGGLVQPGGSLRLSCAVSGYSITSGYYWNWIRQAPGKGLEWVAYI | VH CDR1-CDR3: |
| 2E1_GD6 | | SYDGNNNYNPSLKNRFTISRDTSKNTFYLQMNSLRAEDTAVYYCAREESTMI | 9-11 |
| | | TTAWFAYWGQGTLVTVSS | VH: 213 |
| | VL | DIQMTQSPSSLSASVGDRVTITCRASEKIYSNLAWYQQKPGKAPKLLIYGAT | VL CDR1-CDR3: |
| | | NLPSGVPSRFSGSGSGTDYTLTISSLQPEDFATYYCQHFWDTPYTFGQGTKV | 282, 283, 15 |
| | | EIK | VL: 284 |
| Antibody | VH | EVQLVESGGGLVQPGGSLRLSCAVSGYSITSGYYWNWIRQAPGKGLEWVAYI | VH CDR1-CDR3: |
| 2E1_GA9 | | SYDGNNNYNPSLKNRFTISRDTSKNTFYLQMNSLRAEDTAVYYCAREESTMI | 9-11 |
| | | TTAWFAYWGQGTLVTVSS | VH: 213 |
| | VL | DIQMTQSPSSLSASVGDRVTITCEASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 285, 14, 15 |
| | | EIK | VL: 286 |
| Antibody | VH | EVQLVESGGGLVQPGGSLRLSCAVSGYSITSGYYWNWIRQAPGKGLEWVAYI | VH CDR1-CDR3: |
| 2E1_GD9 | | SYDGNNNYNPSLKNRFTISRDTSKNTFYLQMNSLRAEDTAVYYCAREESTMI | 9-11 |
| | | TTAWFAYWGQGTLVTVSS | VH: 213 |
| | VL | DIQMTQSPSSLSASVGDRVTITCRASENIFSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 287, 14, 15 |
| | | EIK | VL: 288 |
| Antibody | VH | EVQLVESGGGLVQPGGSLRLSCAVSGYSITSGYYWWWIRQAPGKGLEWVAYI | VH CDR1-CDR3: |
| 2E1_GH11 | | SYDGNNNYNPSLKNRFTISRDTSKNTFYLQMNSLRAEDTAVYYCAREESTMI | 289, 290, 11 |
| | | TTAWFAYWGQGTLVTVSS | VH: 291 |
| | VL | DIQMTQSPSSLSASVGDRVTITCNASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 292, 14, 15 |
| | | EIK | VL: 293 |
| Antibody | VH | EVQLVESGGGLVQPGGSLRLSCAVSGYSITSGYYWWWIRQAPGKGLEWVAYI | VH CDR1-CDR3: |
| 2E1_GA1 | | SYDGNNNYNPSLKNRFTISRDTSKNTFYLQMNSLRAEDTAVYYCAREESTMI | 289, 290, 11 |
| | | TTAWFAYWGQGTLVTVSS | VH: 291 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWWWIRQAPGKGLEWVAYI | VH CDR1-CDR3: |
| 2E1_GH4 | | SYDGNNNYNPSLKNRFTISRDTSKNTFYLQMNSLRAEDTAVYYCAREESTMI | 289, 290, 11 |
| | | TTAWFAYWGQGTLVTVSS | VH: 291 |
| | VL | DIQMTQSPSSLSASVGDRVTITCRASENIYSNLAWYQQKPGKAPKLLIYGAT | VL CDR1-CDR3: |
| | | NFASGVPSRFSGSGSGTDYTLTISSLQPEDFATYYCQHFWDTPYTFGQGTKV | 13, 294, 15 |
| | | EIK | VL: 295 |
| Antibody | VH | EVQLVESGGGLVQPGGSLRLSCAVSGYSITSGYYWWWIRQAPGKGLEWVAYI | VH CDR1-CDR3: |
| 2E1_GE6 | | SYDGNNNYNPSLKNRFTISRDTSKNTFYLQMNSLRAEDTAVYYCAREESTMI | 289, 290, 11 |
| | | TTAWFAYWGQGTLVTVSS | VH: 291 |
| | VL | DIQMTQSPSSLSASVGDRVTITCRASENIYSNNAWYQQKPGKAPKLLIYGAT | VL CDR1-CDR3: |
| | | YLASGVPSRFSGSGSGTDYTLTISSLQPEDFATYYCQHFWDTPYTFGQGTKV | 296, 297, 15 |
| | | EIK | VL: 298 |
| Antibody | VH | EVQLVESGGGLVQPGGSLRLSCAVSGYSITSGYYWWWIRQAPGKGLEWVAYI | VH CDR1-CDR3: |
| 2E1_GB7 | | SYDGNNNYNPSLKNRFTISRDTSKNTFYLQMNSLRAEDTAVYYCAREESTMI | 289, 290, 11 |
| | | TTAWFAYWGQGTLVTVSS | VH: 291 |
| | VL | DIQMTQSPSSLSASVGDRVTITCLASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 299, 14, 15 |
| | | EIK | VL: 300 |
| Antibody | VH | EVQLVESGGGLVQPGGSLRLSCAVSGYSITSGYYWWWIRQAPGKGLEWVAYI | VH CDR1-CDR3: |
| 2E1_GB8 | | SYDGNNNYNPSLKNRFTISRDTSKNTFYLQMNSLRAEDTAVYYCAREESTMI | 289, 290, 11 |
| | | TTAWFAYWGQGTLVTVSS | VH: 291 |
| | VL | DIQMTQSPSSLSASVGDRVTITCSASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 301, 14, 15 |
| | | EIK | VL: 302 |
| Antibody | VH | EVQLVESGGGLVQPGGSLRLSCAVSGYSITSGYYWWWIRQAPGKGLEWVAYI | VH CDR1-CDR3: |
| 2E1_GC8 | | SYDGNNNYNPSLKNRFTISRDTSKNTFYLQMNSLRAEDTAVYYCAREESTMI | 289, 290, 11 |
| | | TTAWFAYWGQGTLVTVSS | VH: 291 |
| | VL | DIQMTQSPSSLSASVGDRVTITCRASENIYSNQAWYQQKPGKAPKLLIYGAT | VL CDR1-CDR3: |
| | | NRASGVPSRFSGSGSGTDYTLTISSLQPEDFATYYCQHFWDTPYTFGQGTKV | 303, 304, 15 |
| | | EIK | VL: 305 |
| Antibody | VH | EVQLVESGGGLVQPGGSLRLSCAVSGYSITSGYYWWWIRQAPGKGLEWVAYI | VH CDR1-CDR3: |
| 2E1_GF8 | | SYDGNNNYNPSLKNRFTISRDTSKNTFYLQMNSLRAEDTAVYYCAREESTMI | 289, 290, 11 |
| | | TTAWFAYWGQGTLVTVSS | VH: 291 |
| | VL | DIQMTQSPSSLSASVGDRVTITCRGSENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 306, 14, 15 |
| | | EIK | VL: 307 |
| Antibody | VH | EVQLVESGGGLVQPGGSLRLSCAVSGYSITSGYSWNWIRQAPGKGLEWVAYI | VH CDR1-CDR3: |
| 2E1_GB1 | | SYDGNNNYNPSLKNRFTISRDTSKNTFYLQMNSLRAEDTAVYYCAREESTMI | 308, 290, 11 |
| | | TTAWFAYWGQGTLVTVSS | VH: 309 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYSWNWIRQAPGKGLEWVAYI | VH CDR1-CDR3: |
| 2E1_GF9 | | SYDGNNNYNPSLKNRFTISRDTSKNTFYLQMNSLRAEDTAVYYCAREESTMI | 308, 290, 11 |
| | | TTAWFAYWGQGTLVTVSS | VH: 309 |
| | VL | DIQMTQSPSSLSASVGDRVTITCRASETIYSNLAWYQQKPGKAPKLLIYGAT | VL CDR1-CDR3: |
| | | NLAAGVPSRFSGSGSGTDYTLTISSLQPEDFATYYCQHFWDTPYTFGQGTKV | 310, 311, 15 |
| | | EIK | VL: 312 |
| Antibody | VH | EVQLVESGGGLVQPGGSLRLSCAVSGYSITSGYSWNWIRQAPGKGLEWVAYI | VH CDR1-CDR3: |
| 2E1_GE11 | | SYDGNNNYNPSLKNRFTISRDTSKNTFYLQMNSLRAEDTAVYYCAREESTMI | 308, 290, 11 |
| | | TTAWFAYWGQGTLVTVSS | VH: 309 |
| | VL | DIQMTQSPSSLSASVGDRVTITCGASENIYSNLAWYQQKPGKAPKLLIYGAT | VL CDR1-CDR3: |
| | | ELASGVPSRFSGSGSGTDYTLTISSLQPEDFATYYCQHFWDTPYTFGQGTKV | 313, 314, 15 |
| | | EIK | VL: 315 |
| Antibody | VH | EVQLVESGGGLVQPGGSLRLSCAVSGYSITSGYAWNWIRQAPGKGLEWVAYI | VH CDR1-CDR3: |
| 2E1_GG1 | | SYDGNNNYNPSLKNRFTISRDTSKNTFYLQMNSLRAEDTAVYYCAREESTMI | 316, 290, 11 |
| | | TTAWFAYWGQGTLVTVSS | VH: 317 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYAWNWIRQAPGKGLEWVAYI | VH CDR1-CDR3: |
| 2E1_GA4 | | SYDGNNNYNPSLKNRFTISRDTSKNTFYLQMNSLRAEDTAVYYCAREESTMI | 316, 290, 11 |
| | | TTAWFAYWGQGTLVTVSS | VH: 317 |
| | VL | DIQMTQSPSSLSASVGDRVTITCVASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 318, 14, 15 |
| | | EIK | VL: 319 |
| Antibody | VH | EVQLVESGGGLVQPGGSLRLSCAVSGGSITSGYYWWWIRQAPGKGLEWVAYI | VH CDR1-CDR3: |
| 2E1_GD5 | | SYDGNNNYNPSLKNRFTISRDTSKNTFYLQMNSLRAEDTAVYYCAREESTMI | 320, 290, 11 |
| | | TTAWFAYWGQGTLVTVSS | VH: 321 |
| | VL | DIQMTQSPSSLSASVGDRVTITCRASENIYSNLAWYQQKPGKAPKLLIYGAT | VL CDR1-CDR3: |
| | | NLAKGVPSRFSGSGSGTDYTLTISSLQPEDFATYYCQHFWDTPYTFGQGTKV | 13, 322, 15 |
| | | EIK | VL: 323 |
| Antibody | VH | EVQLVESGGGLVQPGGSLRLSCAVSGGSITSGYYWWWIRQAPGKGLEWVAYI | VH CDR1-CDR3: |
| 2E1_GE8 | | SYDGNNNYNPSLKNRFTISRDTSKNTFYLQMNSLRAEDTAVYYCAREESTMI | 320, 290, 11 |
| | | TTAWFAYWGQGTLVTVSS | VH: 321 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYHWNWIRQAPGKGLEWVAYI | VH CDR1-CDR3: |
| 2E1_GH6 | | SYDGNNNYNPSLKNRFTISRDTSKNTFYLQMNSLRAEDTAVYYCAREESTMI | 324, 290, 11 |
| | | TTAWFAYWGQGTLVTVSS | VH: 325 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| AFH6 | | SYDGNTNYNPSLKNRFTISRDTSKNTFYLQMNSLRAEDTAVYYCAREESTMI | 9, 326, 11 |
| | | TTAWFAYWGQGTLVTVSS | VH: 230 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQQFWDTPYTFGQGTKV | 13, 14, 327 |
| | | EIK | VL: 328 |
| Antibody | VH | EVQLVESGGGLVQPGGSLRLSCAVSGYSITSGYSWNWIRQAPGKGLEWVAYI | VH CDR1-CDR3: |
| AGH6 | | SYDGNNNYNPSLKNRFTISRDTSKNTFYLQMNSLRAEDTAVYYCAREESTMI | 308, 290, 11 |
| | | TTAWFAYWGQGTLVTVSS | VH: 309 |
| | VL | DIQMTQSPSSLSASVGDRVTITCQASENIYSNLAWYQQKPGKAPKLLIYGAT | VL CDR1-CDR3: |
| | | YLASGVPSRFSGSGSGTDYTLTISSLQPEDFATYYCQHFWDTPYTFGQGTKV | 329, 297, 15 |
| | | EIK | VL: 330 |
| Antibody | VH | EVQLVESGGGLVQPGGSLRLSCAVSGYSITSGYYWSWIRQAPGKGLEWVAYI | VH CDR1-CDR3: |
| AGH4 | | SYDGNNNYNPSLKNRFTISRDTSKNTFYLQMNSLRAEDTAVYYCAREESTMI | 331, 290, 11 |
| | | TTAWFAYWGQGTLVTVSS | VH: 332 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| 2AFH5 | | SYDGNVNYNPSLKNRFTISRDTSKNTFYLQMNSLRAEDTAVYYCAREESTMI | 9, 333, 11 |
| | | TTAWFAYWGQGTLVTVSS | VH: 218 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDSPYTFGQGTKV | 13, 14, 334 |
| | | EIK | VL: 335 |
| Antibody | VH | EVQLVESGGGLVQPGGSLRLSCAVSGYSITSGYYWNWIRQAPGKGLEWVATI | VH CDR1-CDR3: |
| 2AFH10 | | SYDGNPNYNPSLKNRFTISRDTSKNTFYLQMNSLRAEDTAVYYCAREESTMI | 9, 336, 11 |
| | | TTAWFAYWGQGTLVTVSS | VH: 337 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| 2AGH2 | | SYDGNNNYNPSLKNRFTISRDTSKNTFYLQMNSLRAEDTAVYYCAREESTMI | 9, 290, 11 |
| | | TTAWFAYWGQGTLVTVSS | VH: 213 |
| | VL | DIQMTQSPSSLSASVGDRVTITCRASENIYSNLAWYQQKPGKAPKLLIYGAT | VL CDR1-CDR3: |
| | | NLAVGVPSRFSGSGSGTDYTLTISSLQPEDFATYYCQHFWDTPYTFGQGTKV | 13, 338, 15 |
| | | EIK | VL: 339 |
| Antibody | VH | EVQLVESGGGLVQPGGSLRLSCAVSGYSITSGYYWNWIRQAPGKGLEWVAYI | VH CDR1-CDR3: |
| 3AFA2 | | SYDGNVNYNPSLKNRFTISRDTSKNTFYLQMNSLRAEDTAVYYCAREESTMI | 9, 333, 11 |
| | | TTAWFAYWGQGTLVTVSS | VH: 218 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQQFWDTPYTFGQGTKV | 13, 14 ,327 |
| | | EIK | VL: 328 |
| Antibody | VE | EVQLVESGGGLVQPGGSLRLSCAVSGYSITSGYYWNWIRQAPGKGLEWVAYI | VH CDR1-CDR3: |
| 3AFH9 | | SYDGNINYNPSLKNRFTISRDTSKNTFYLQMNSLRAEDTAVYYCAREESTMI | 9, 340, 11 |
| | | TTAWFAYWGQGTLVTVSS | VH: 244 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYRFGQGTKV | 13, 14, 21 |
| | | EIK | VL: 222 |
| Antibody | VH | EVQLVESGGGLVQPGGSLRLSCAVSGYSITSGYYWNWIRQAPGKGLEWVAYI | VH CDR1-CDR3: |
| 3AFA11 | | SYDGNSNYNPSLKNRFTISRDTSKNTFYLQMNSLRAEDTAVYYCAREESTMI | 9, 341, 11 |
| | | TTAWFAYWGQGTLVTVSS | VH: 342 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| 3AFG1 | | SYDGNNWYNPSLKNRFTISRDTSKNTFYLQMNSLRAEDTAVYYCAREESTMI | 9, 343, 344 |
| | | TTGWFAYWGQGTLVTVSS | VH: 345 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVAYI | VH CDR1-CDR3: |
| 4AFF8 | | SYDGNVNYNPSLKNRFTISRDTSKNTFYLQMNSLRAEDTAVYYCAREESTMI | 9, 333, 11 |
| | | TTAWFAYWGQGTLVTVSS | VH: 218 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDLPYTFGQGTKV | 13, 14, 346 |
| | | EIK | VL: 347 |
| Antibody | VH | EVQLVESGGGLVQPGGSLRLSCAVSGYSITSGYYWNWIRQAPGKGLEWVAYI | VH CDR1-CDR3: |
| 4AFH3 | | SYDGNWNYNPSLKNRFTISRDTSKNTFYLQMNSLRAEDTAVYYCAREESTMF | 9, 348, 349 |
| | | TTAWFAYWGQGTLVTVSS | VH: 350 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDTPYTFGQGTKV | 13-15 |
| | | EIK | VL: 208 |
| Antibody | VH | EVQLVESGGGLVQPGGSLRLSCAVSGYSITSGYYWNWIRQAPGKGLEWVASI | VH CDR1-CDR3: |
| 4AFH7 | | SYDGNNNYNPSLKNRFTISRDTSKNTFYLQMNSLRAEDTAVYYCAREESTMI | 9, 351, 352 |
| | | TTAWFGYWGQGTLVTVSS | VH: 353 |
| | VL | DIQMTQSPSSLSASVGDRVTITCRASENIYSNLAWYQQKPGKAPKLLIYGAT | VL CDR1-CDR3: |
| | | NLASGVPSRFSGSGSGTDYTLTISSLQPEDFATYYCQHFWDYPYTFGQGTKV | 13, 14, 247 |
| | | EIK | VL: 248 |

In some examples, the anti-LILRB2 antibody is h#2E1.

In some instances, the humanized antibody may be derived from the parent antibody 9B36. Such a humanized antibody may comprise the same heavy chain and light chain CDRs as those of antibody 9B36 (see Table 1 above and Table 3 below) and the heavy chain and light chain framework regions (HC FR1-F4 and LC FR1-FR4) provided in Table 3 below.

TABLE 3
Humanized Variants of Clone 9B6

| | | | SEQ |
|---|---|---|---|
| Antibody | | SEQUENCE | ID NO |
| HC FR1 Motif | | EVQLVESGGZ₁LVQPGGSLRLSCAZ₂S, in which Z₁ is D or G, and | 354 |
| | | Z₂ is A, I, or V | |
| HC CDR1 | | GFTFSNFGMH | 73 |
| HC FR2 Motif | | WZ₃RQAPGKGLEWVA, in which Z₃ is I or V | 193 |
| HC CDR2 | | YISSGSGTIYYSDTVRG | 74 |
| HC FR3 Motif | | RZ₄TISRDZ₅SKNTLYLQMNSLRAEDTAVYYCZ₆R, in which Z₄ is A, | 355 |
| | | F, I, L S, or V, Z₅ is D or N, and Z₆ is A or S | |
| HC CDR3 | | SDYDGDWFFDV | 75 |
| HC FR4 Motif | | WGQGTLVTVSS | 197 |
| LC FR1 Motif | | DIQZ₇TQSPSSLSASVGDRVTITC, in which Z₇ is L or M | 198 |
| LC CDR1 | | KASQDVTTAVA | 77 |
| LC FR2 Motif | | WZ₈QQKPGKAPKLLIY, in which Z₈ is F or Y | 356 |
| LC CDR2 | | SASFRYT | 78 |
| LC FR3 Motif | | GVPSRFSGSGSGTDZ₉TLTISSLOPEDZ₁₀ATYYC, in which Z₉ is F | 357 |
| | | or Y, and Z₁₀ is F or L | |
| LC CDR3 | | QQHFTTPLT | 79 |
| LC FR4 Motif | | FGQGTKVEIK | 204 |
| Antibody | VH | EVQLVESGGGLVQPGGSLRLSCAASGFTFSNFGMHWVRQAPGKGLEWVAYISSG | 358 |
| h#12a | | SGTIYYSDTVRGRFTISRDDSKNTLYLQMNSLRAEDTAVYYCARSDYDGDWFFD | |
| | | VWGQGTLVTVSS | |
| | VL | DIQMTQSPSSLSASVGDRVTITCKASQDVTTAVAWYQQKPGKAPKLLIYSASFR | 359 |
| | | YTGVPSRFSGSGSGTDFTLTISSLQPEDFATYYCQQHFTTPLTFGQGTKVEIK | |
| Antibody | VH | EVQLVESGGGLVQPGGSLRLSCAASGFTFSNFGMHWVRQAPGKGLEWVAYISSG | 360 |
| h#12b | | SGTIYYSDTVRGRVTISRDNSKNTLYLQMNSLRAEDTAVYYCARSDYDGDWFFD | |
| | | VWGQGTLVTVSS | |
| | VL | DIQLTQSPSSLSASVGDRVTITCKASQDVTTAVAWYQQKPGKAPKLLIYSASER | 361 |
| | | YTGVPSRFSGSGSGTDFTLTISSLQPEDFATYYCQQHFTTPLTFGQGTKVEIK | |
| Antibody | VH | EVQLVESGGGLVQPGGSLRLSCATSGFTFSNFGMHWVRQAPGKGLEWVAYISSG | 362 |
| h#12c | | SGTIYYSDTVRGRFTISRDNSKNTLYLQMNSLRAEDTAVYYCARSDYDGDWFFD | |
| | | VWGQGTLVTVSS | |
| | VL | DIQMTQSPSSLSASVGDRVTITCKASQDVTTAVAWFQQKPGKAPKLLIYSASFR | 363 |
| | | YTGVPSRFSGSGSGTDFTLTISSLQPEDFATYYCQQHFTTPLTFGQGTKVEIK | |
| Antibody | VH | EVQLVESGGGLVQPGGSLRLSCAASGFTFSNFGMHWVRQAPGKGLEWVAYISSG | 364 |
| h#12d | | SGTIYYSDTVRGRFTISRDNSKNTLYLQMNSLRAEDTAVYYCARSDYDGDWFFD | |
| | | VWGQGTLVTVSS | |
| | VL | DIQMTQSPSSLSASVGDRVTITCKASQDVTTAVAWYQQKPGKAPKLLIYSASFR | 365 |
| | | YTGVPSRFSGSGSGTDYTLTISSLQPEDFATYYCQQHFTTPLTFGQGTKVEIK | |
| Antibody | VH | EVQLVESGGGLVQPGGSLRLSCAASGFTFSNFGMHWVRQAPGKGLEWVAYISSG | 364 |
| h#12e | | SGTIYYSDTVRGRFTISRDNSKNTLYLQMNSLRAEDTAVYYCARSDYDGDWFFD | |
| | | VWGQGTLVTVSS | |
| | VL | DIQMTQSPSSLSASVGDRVTITCKASQDVTTAVAWYQQKPGKAPKLLIYSASFR | 366 |
| | | YTGVPSRFSGSGSGTDFTLTISSLQPEDLATYYCQQHFTTPLTFGQGTKVEIK | |
| Antibody | VH | EVQLVESGGGLVQPGGSLRLSCATSGFTFSNFGMHWIRQAPGKGLEWVAYISSG | 367 |
| h#12f | | SGTIYYSDTVRGRFTISRDNSKNTLYLQMNSLRAEDTAVYYCARSDYDGDWFFD | |
| | | VWGQGTLVTVSS | |
| | VL | DIQMTQSPSSLSASVGDRVTITCKASQDVTTAVAWFQQKPGKAPKLLIYSASFR | 368 |
| | | YTGVPSRFSGSGSGTDFTLTISSLQPEDLATYYCQQHFTTPLTFGQGTKVEIK | |
| Antibody | VH | EVQLVESGGDLVQPGGSLRLSCAASGFTFSNFGMHWVRQAPGKGLEWVAYISSG | 369 |
| h#12g | | SGTIYYSDTVRGRFTISRDNSKNTLYLQMNSLRAEDTAVYYCARSDYDGDWFFD | |
| | | VWGQGTLVTVSS | |
| | VL | DIQMTQSPSSLSASVGDRVTITCKASQDVTTAVAWFQQKPGKAPKLLIYSASFR | 363 |
| | | YTGVPSRFSGSGSGTDFTLTISSLQPEDFATYYCQQHFTTPLTFGQGTKVEIK | |
| Antibody | VH | EVQLVESGGGLVQPGGSLRLSCAASGFTFSNFGMHWVRQAPGKGLEWVAYISSG | 364 |
| h#12h | | SGTIYYSDTVRGRFTISRDNSKNTLYLQMNSLRAEDTAVYYCARSDYDGDWFFD | |
| | | VWGQGTLVTVSS | |
| | VL | DIQMTQSPSSLSASVGDRVTITCKASQDVTTAVAWFQQKPGKAPKLLIYSASFR | 363 |
| | | YTGVPSRFSGSGSGTDFTLTISSLQPEDFATYYCQQHFTTPLTFGQGTKVEIK | |
| Antibody | VH | EVQLVESGGGLVQPGGSLRLSCAASGFTFSNFGMHWVRQAPGKGLEWVAYISSG | 364 |
| h#12i | | SGTIYYSDTVRGRFTISRDNSKNTLYLQMNSLRAEDTAVYYCARSDYDGDWFFD | |
| | | VWGQGTLVTVSS | |
| | VL | DIQMTQSPSSLSASVGDRVTITCKASQDVTTAVAWFQQKPGKAPKLLIYSASFR | 368 |
| | | YTGVPSRFSGSGSGTDFTLTISSLQPEDLATYYCQQHFTTPLTFGQGTKVEIK | |
| Antibody | VH | EVQLVESGGGLVQPGGSLRLSCATSGFTFSNFGMHWVRQAPGKGLEWVAYISSG | 362 |
| h#12j | | SGTIYYSDTVRGRFTISRDNSKNTLYLQMNSLRAEDTAVYYCARSDYDGDWFFD | |
| | | VWGQGTLVTVSS | |
| | VL | DIQMTQSPSSLSASVGDRVTITCKASQDVTTAVAWYQQKPGKAPKLLIYSASFR | 359 |
| | | YTGVPSRFSGSGSGTDFTLTISSLQPEDFATYYCQQHFTTPLTFGQGTKVEIK | |
| Antibody | VH | EVQLVESGGGLVQPGGSLRLSCAASGFTFSNFGMHWVRQAPGKGLEWVAYISSG | 360 |
| h#12k | | SGTIYYSDTVRGRVTISRDNSKNTLYLQMNSLRAEDTAVYYCARSDYDGDWFFD | |
| | | VWGQGTLVTVSS | |
| | VL | DIQMTQSPSSLSASVGDRVTITCKASQDVTTAVAWYQQKPGKAPKLLIYSASFR | 359 |
| | | YTGVPSRFSGSGSGTDFTLTISSLQPEDFATYYCQQHFTTPLTFGQGTKVEIK | |
| Antibody | VH | EVQLVESGGGLVQPGGSLRLSCAASGFTFSNFGMHWVRQAPGKGLEWVAYISSG | 370 |
| h#12l | | SGTIYYSDTVRGRITISRDNSKNTLYLQMNSLRAEDTAVYYCARSDYDGDWFFD | |
| | | VWGQGTLVTVSS | |
| | VL | DIQMTQSPSSLSASVGDRVTITCKASQDVTTAVAWYQQKPGKAPKLLIYSASFR | 359 |
| | | YTGVPSRFSGSGSGTDFTLTISSLQPEDFATYYCQQHFTTPLTFGQGTKVEIK | |
| Antibody | VH | EVQLVESGGGLVQPGGSLRLSCATSGFTFSNFGMHWVRQAPGKGLEWVAYISSG | 371 |
| h#12m | | SGTIYYSDTVRGRLTISRDNSKNTLYLQMNSLRAEDTAVYYCARSDYDGDWFFD | |
| | | VWGQGTLVTVSS | |
| | VL | DIQMTQSPSSLSASVGDRVTITCKASQDVTTAVAWYQQKPGKAPKLLIYSASFR | 359 |
| | | YTGVPSRFSGSGSGTDFTLTISSLQPEDFATYYCQQHFTTPLTFGQGTKVEIK | |
| Antibody | VH | EVQLVESGGGLVQPGGSLRLSCATSGFTFSNFGMHWVRQAPGKGLEWVAYISSG | 372 |
| h#12n | | SGTIYYSDTVRGRATISRDNSKNTLYLQMNSLRAEDTAVYYCSRSDYDGDWFFD | |
| | | VWGQGTLVTVSS | |
| | VL | DIQMTQSPSSLSASVGDRVTITCKASQDVTTAVAWYQQKPGKAPKLLIYSASFR | 359 |
| | | YTGVPSRFSGSGSGTDFTLTISSLQPEDFATYYCQQHFTTPLTFGQGTKVEIK | |
| Antibody | VH | EVQLVESGGGLVQPGGSLRLSCAASGFTFSNFGMHWVRQAPGKGLEWVAYISSG | 364 |
| h#12A1 | | SGTIYYSDTVRGRFTISRDNSKNTLYLQMNSLRAEDTAVYYCARSDYDGDWFFD | |
| | | VWGQGTLVTVSS | |
| | VL | DIQMTQSPSSLSASVGDRVTITCKASQDVTTAVAWYQQKPGKAPKLLIYSASFR | 359 |
| | | YTGVPSRFSGSGSGTDFTLTISSLQPEDFATYYCQQHFTTPLTFGQGTKVEIK | |
| Antibody | VH | EVQLVESGGGLVQPGGSLRLSCAASGFTFSNFGMHWVRQAPGKGLEWVAYISSG | 364 |
| h#12B2 | | SGTIYYSDTVRGRFTISRDNSKNTLYLQMNSLRAEDTAVYYCARSDYDGDWFFD | |
| | | VWGQGTLVTVSS | |
| | VL | DIQLTQSPSSLSASVGDRVTITCKASQDVTTAVAWYQQKPGKAPKLLIYSASFR | 361 |
| | | YTGVPSRFSGSGSGTDFTLTISSLQPEDFATYYCQQHFTTPLTFGQGTKVEIK | |
| Antibody | VH | EVQLVESGGGLVQPGGSLRLSCAASGFTFSNFGMHWVRQAPGKGLEWVAYISSG | 364 |
| h#12D3 | | SGTIYYSDTVRGRFTISRDNSKNTLYLQMNSLRAEDTAVYYCARSDYDGDWFFD | |
| | | VWGQGTLVTVSS | |
| | VL | DIQLTQSPSSLSASVGDRVTITCKASQDVTTAVAWFQQKPGKAPKLLIYSASFR | 373 |
| | | YTGVPSRFSGSGSGTDYTLTISSLQPEDLATYYCQQHFTTPLTFGQGTKVEIK | |
| Antibody | VH | EVQLVESGGGLVQPGGSLRLSCAASGFTFSNFGMHWVRQAPGKGLEWVAYISSG | 374 |
| h#12G1 | | SGTIYYSDTVRGRLTISRDNSKNTLYLQMNSLRAEDTAVYYCARSDYDGDWFFD | |
| | | VWGQGTLVTVSS | |
| | VL | DIQMTQSPSSLSASVGDRVTITCKASQDVTTAVAWYQQKPGKAPKLLIYSASFR | 359 |
| | | YTGVPSRFSGSGSGTDFTLTISSLQPEDFATYYCQQHFTTPLTFGQGTKVEIK | |
| Antibody | VH | EVQLVESGGGLVQPGGSLRLSCAASGFTFSNFGMHWVRQAPGKGLEWVAYISSG | 375 |
| h#12G3 | | SGTIYYSDTVRGRSTISRDNSKNTLYLQMNSLRAEDTAVYYCARSDYDGDWFFD | |
| | | VWGQGTLVTVSS | |
| | VL | DIQMTQSPSSLSASVGDRVTITCKASQDVTTAVAWYQQKPGKAPKLLIYSASER | 359 |
| | | YTGVPSRFSGSGSGTDFTLTISSLQPEDFATYYCQQHFTTPLTFGQGTKVEIK | |
| Antibody | VH | EVQLVESGGGLVQPGGSLRLSCAASGFTFSNFGMHWVRQAPGKGLEWVAYISSG | 360 |
| h#12G4 | | SGTIYYSDTVRGRVTISRDNSKNTLYLQMNSLRAEDTAVYYCARSDYDGDWFFD | |
| | | VWGQGTLVTVSS | |
| | VL | DIQMTQSPSSLSASVGDRVTITCKASQDVTTAVAWFQQKPGKAPKLLIYSASFR | 368 |
| | | YTGVPSRFSGSGSGTDFTLTISSLQPEDLATYYCQQHFTTPLTFGQGTKVEIK | |
| Antibody | VH | EVQLVESGGGLVQPGGSLRLSCAASGFTFSNFGMHWIRQAPGKGLEWVAYISSG | 376 |
| h#12H1 | | SGTIYYSDTVRGRFTISRDNSKNTLYLQMNSLRAEDTAVYYCARSDYDGDWFFD | |
| | | VWGQGTLVTVSS | |
| | VL | DIQMTQSPSSLSASVGDRVTITCKASQDVTTAVAWYQQKPGKAPKLLIYSASFR | 366 |
| | | YTGVPSRFSGSGSGTDFTLTISSLQPEDLATYYCQQHFTTPLTFGQGTKVEIK | |
| Antibody | VH | EVQLVESGGGLVQPGGSLRLSCAASGFTFSNFGMHWIRQAPGKGLEWVAYISSG | 376 |
| h#12A2 | | SGTIYYSDTVRGRFTISRDNSKNTLYLQMNSLRAEDTAVYYCARSDYDGDWFFD | |
| | | VWGQGTLVTVSS | |
| | VL | DIQLTQSPSSLSASVGDRVTITCKASQDVTTAVAWYQQKPGKAPKLLIYSASFR | 361 |
| | | YTGVPSRFSGSGSGTDFTLTISSLQPEDFATYYCQQHFTTPLTFGQGTKVEIK | |
| Antibody | VH | EVQLVESGGGLVQPGGSLRLSCATSGFTFSNFGMHWVRQAPGKGLEWVAYISSG | 377 |
| h#12C2 | | SGTIYYSDTVRGRITISRDNSKNTLYLQMNSLRAEDTAVYYCSRSDYDGDWFFD | |
| | | VWGQGTLVTVSS | |
| | VL | DIQMTQSPSSLSASVGDRVTITCKASQDVTTAVAWFQQKPGKAPKLLIYSASFR | 363 |
| | | YTGVPSRFSGSGSGTDFTLTISSLQPEDFATYYCQQHFTTPLTFGQGTKVEIK | |
| Antibody | VH | EVQLVESGGGLVQPGGSLRLSCAVSGFTFSNFGMHWVRQAPGKGLEWVAYISSG | 378 |
| h#12G2 | | SGTIYYSDTVRGRFTISRDNSKNTLYLQMNSLRAEDTAVYYCARSDYDGDWFFD | |
| | | VWGQGTLVTVSS | |
| | VL | DIQMTQSPSSLSASVGDRVTITCKASQDVTTAVAWYQQKPGKAPKLLIYSASFR | 366 |
| | | YTGVPSRFSGSGSGTDFTLTISSLQPEDLATYYCQQHFTTPLTFGQGTKVEIK | |
| Antibody | VH | EVQLVESGGGLVQPGGSLRLSCAVSGFTFSNFGMHWVRQAPGKGLEWVAYISSG | 378 |
| h#12C3 | | SGTIYYSDTVRGRFTISRDNSKNTLYLQMNSLRAEDTAVYYCARSDYDGDWFFD | |
| | | VWGQGTLVTVSS | |
| | VL | DIQMTQSPSSLSASVGDRVTITCKASQDVTTAVAWYQQKPGKAPKLLIYSASFR | 359 |
| | | YTGVPSRFSGSGSGTDFTLTISSLQPEDFATYYCQQHFTTPLTFGQGTKVEIK | |
| Antibody | VH | EVQLVESGGGLVQPGGSLRLSCATSGFTFSNFGMHWIRQAPGKGLEWVAYISSG | 367 |
| h#12F6 | | SGTIYYSDTVRGRFTISRDNSKNTLYLQMNSLRAEDTAVYYCARSDYDGDWFFD | |
| | | VWGQGTLVTVSS | |
| | VL | DIQMTQSPSSLSASVGDRVTITCKASQDVTTAVAWYQQKPGKAPKLLIYSASFR | 359 |
| | | YTGVPSRFSGSGSGTDFTLTISSLQPEDFATYYCQQHFTTPLTFGQGTKVEIK | |

In some instances, the humanized antibody may be derived from the parent antibody 14F1. Such a humanized antibody may comprise the same heavy chain and light chain CDRs as those of antibody 14F1 (see Table 1 above and Table 4 below) and the heavy chain and light chain framework regions (HC FR1-F4 and LC FR1-FR4) provided in Table 4 below.

TABLE 4
Humanized Variants of Clone 14F1

| | | | SEQ ID |
|---|---|---|---|
| Antibody | | SEQUENCE | NO |
| HC FR1 Motif | | EVQLVESGGGLVQPGGSLRLSCAZ₁S, in which Z₁ is A, I, or V | 379 |
| HC CDR1 | | GFSLTTYGVH | 141 |
| HC FR2 Motif | | WZ₂RQAPGKGLEWVA, in which Z₂ is I or V | 193 |
| HC CDR2 | | VITGGSTNYNSALMS | 380 |
| HC FR3 Motif | | RZ₃TISZ₄DZ₅SKZ₆TZ₇YLQMNSLRAEDTAVYYCAZ₈, in which Z₃ is | 381 |
| | | A, F, I, T, or V, Z₄ is K or R, Z₅ is D or N, Z₆ is | |
| | | A, L, or V, Z₇ is A, L, or V, and Z₈ is K or R | |
| HC CDR3 | | GGYGHTMDY | 143 |
| HC FR4 Motif | | WGQGTLVTVSS | 197 |
| LC FR1 Motif | | DIQZ₉TQSPSSLSASVGDRVTITC, in which Z₉ is L or M | 198 |
| LC CDR1 | | KASQDVSIAVA | 131 |
| LC FR2 Motif | | WYQQKPGKAPKLLIY | 200 |
| LC CDR2 | | SASYLYS | 382 |
| LC FR3 Motif | | GVPSRFSGSGSGTDFTLTISSLQPEDZ₁₀ATYYC, in which Z₁₀ is F | 383 |
| | | or L | |
| LC CDR3 | | QQHYSTPFT | 95 |
| LC FR4 Motif | | FGQGTKVEIK | 204 |
| Antibody | VH | EVQLVESGGGLVQPGGSLRLSCAASGFSLTTYGVHWVRQAPGKGLEWVAVIW | 384 |
| | | TGGSTNYNSALMSRFTISRDDSKNTLYLQMNSLRAEDTAVYYCARGGYGHTM | |
| | | DYWGQGTLVTVSS | |
| h#22a | VL | DIQMTQSPSSLSASVGDRVTITCKASQDVSIAVAWYQQKPGKAPKLLIYSAS | 385 |
| | | YLYSGVPSRFSGSGSGTDFTLTISSLQPEDFATYYCQQHYSTPFTFGQGTKV | |
| | | EIK | |
| Antibody | VH | EVQLVESGGGLVQPGGSLRLSCAASGFSLTTYGVHWVRQAPGKGLEWVAVIW | 386 |
| | | TGGSTNYNSALMSRFTISKDDSKNTLYLQMNSLRAEDTAVYYCARGGYGHTM | |
| | | DYWGQGTLVTVSS | |
| h#22C1 | VL | DIQMTQSPSSLSASVGDRVTITCKASQDVSIAVAWYQQKPGKAPKLLIYSAS | 385 |
| | | YLYSGVPSRFSGSGSGTDFTLTISSLQPEDFATYYCQQHYSTPFTFGQGTKV | |
| | | EIK | |
| Antibody | VH | EVQLVESGGGLVQPGGSLRLSCAASGFSLTTYGVHWVRQAPGKGLEWVAVIW | 386 |
| h#22H1 | | TGGSTNYNSALMSRFTISKDDSKNTLYLQMNSLRAEDTAVYYCARGGYGHTM | |
| | | DYWGQGTLVTVSS | |
| | VL | DIQMTQSPSSLSASVGDRVTITCKASQDVSIAVAWYQQKPGKAPKLLIYSAS | 387 |
| | | YLYSGVPSRFSGSGSGTDFTLTISSLQPEDLATYYCQQHYSTPFTFGQGTKV | |
| | | EIK | |
| Antibody | VH | EVQLVESGGGLVQPGGSLRLSCAASGFSLTTYGVHWVRQAPGKGLEWVAVIW | 388 |
| h#22B2 | | TGGSTNYNSALMSRFTISKDDSKNTVYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 385 |
| | | YLYSGVPSRFSGSGSGTDFTLTISSLQPEDFATYYCQQHYSTPFTFGQGTKV | |
| | | EIK | |
| Antibody | VH | EVQLVESGGGLVQPGGSLRLSCAVSGFSLTTYGVHWVRQAPGKGLEWVAVIW | 389 |
| h#22A1 | | TGGSTNYNSALMSRFTISKDDSKNTLYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 387 |
| | | YLYSGVPSRFSGSGSGTDFTLTISSLQPEDLATYYCQQHYSTPFTFGQGTKV | |
| | | EIK | |
| Antibody | VH | EVQLVESGGGLVQPGGSLRLSCAVSGFSLTTYGVHWVRQAPGKGLEWVAVIW | 389 |
| h#22A5 | | TGGSTNYNSALMSRFTISKDDSKNTLYLQMNSLRAEDTAVYYCAKGGYGHTM | |
| | | DYWGQGTLVTVSS | |
| | VL | DIQLTQSPSSLSASVGDRVTITCKASQDVSIAVAWYQQKPGKAPKLLIYSAS | 390 |
| | | YLYSGVPSRFSGSGSGTDFTLTISSLQPEDFATYYCQQHYSTPFTFGQGTKV | |
| | | EIK | |
| Antibody | VH | EVQLVESGGGLVQPGGSLRLSCAVSGFSLTTYGVHWVRQAPGKGLEWVAVIW | 391 |
| h#22A2 | | TGGSTNYNSALMSRTTISKDNSKNTVYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 385 |
| | | YLYSGVPSRFSGSGSGTDFTLTISSLQPEDFATYYCQQHYSTPFTFGQGTKV | |
| | | EIK | |
| Antibody | VH | EVQLVESGGGLVQPGGSLRLSCAVSGFSLTTYGVHWVRQAPGKGLEWVAVIW | 392 |
| h#22D2 | | TGGSTNYNSALMSRATISKDNSKNTVYLQMNSLRAEDTAVYYCARGGYGHTM | |
| | | DYWGQGTLVTVSS | |
| | VL | DIQMTQSPSSLSASVGDRVTITCKASQDVSIAVAWYQQKPGKAPKLLIYSAS | 387 |
| | | YLYSGVPSRFSGSGSGTDFTLTISSLQPEDLATYYCQQHYSTPFTFGQGTKV | |
| | | EIK | |
| Antibody | VH | EVQLVESGGGLVQPGGSLRLSCAVSGFSLTTYGVHWVRQAPGKGLEWVAVIW | 393 |
| h#22E2 | | TGGSTNYNSALMSRFTISKDDSKNTAYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 387 |
| | | YLYSGVPSRFSGSGSGTDFTLTISSLQPEDLATYYCQQHYSTPFTFGQGTKV | |
| | | EIK | |
| Antibody | VH | EVQLVESGGGLVQPGGSLRLSCAVSGFSLTTYGVHWVRQAPGKGLEWVAVIW | 394 |
| h#22G2 | | TGGSTNYNSALMSRFTISKDDSKSTAYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 387 |
| | | YLYSGVPSRFSGSGSGTDFTLTISSLQPEDLATYYCQQHYSTPFTFGQGTKV | |
| | | EIK | |
| Antibody | VH | EVQLVESGGGLVQPGGSLRLSCAVSGFSLTTYGVHWVRQAPGKGLEWVAVIW | 395 |
| h#22H2 | | TGGSTNYNSALMSRVTISKDDSKNTVYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 387 |
| | | YLYSGVPSRFSGSGSGTDFTLTISSLQPEDLATYYCQQHYSTPFTFGQGTKV | |
| | | EIK | |
| Antibody | VH | EVQLVESGGGLVQPGGSLRLSCAVSGFSLTTYGVHWVRQAPGKGLEWVAVIW | 395 |
| h#22H4 | | TGGSTNYNSALMSRVTISKDDSKNTVYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 385 |
| | | YLYSGVPSRFSGSGSGTDFTLTISSLQPEDFATYYCQQHYSTPFTFGQGTKV | |
| | | EIK | |
| Antibody | VH | EVQLVESGGGLVQPGGSLRLSCAVSGFSLTTYGVHWVRQAPGKGLEWVAVIW | 396 |
| h#22C3 | | TGGSTNYNSALMSRFTISKDDSKNTVYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 387 |
| | | YLYSGVPSRFSGSGSGTDFTLTISSLQPEDLATYYCQQHYSTPFTFGQGTKV | |
| | | EIK | |
| Antibody | VH | EVQLVESGGGLVQPGGSLRLSCAVSGFSLTTYGVHWVRQAPGKGLEWVAVIW | 396 |
| h#22G4 | | TGGSTNYNSALMSRFTISKDDSKNTVYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 385 |
| | | YLYSGVPSRFSGSGSGTDFTLTISSLQPEDFATYYCQQHYSTPFTFGQGTKV | |
| | | EIK | |
| Antibody | VH | EVQLVESGGGLVQPGGSLRLSCAVSGFSLTTYGVHWVRQAPGKGLEWVAVIW | 397 |
| h#22F3 | | TGGSTNYNSALMSRATISKDDSKNTVYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 387 |
| | | YLYSGVPSRFSGSGSGTDFTLTISSLQPEDLATYYCQQHYSTPFTFGQGTKV | |
| | | EIK | |
| Antibody | VH | EVQLVESGGGLVQPGGSLRLSCAVSGFSLTTYGVHWVRQAPGKGLEWVAVIW | 398 |
| h#22H3 | | TGGSTNYNSALMSRITISKDDSKSTVYLQMNSLRAEDTAVYYCARGGYGHTM | |
| | | DYWGQGTLVTVSS | |
| | VL | DIQMTQSPSSLSASVGDRVTITCKASQDVSIAVAWYQQKPGKAPKLLIYSAS | 387 |
| | | YLYSGVPSRFSGSGSGTDFTLTISSLQPEDLATYYCQQHYSTPFTFGQGTKV | |
| | | EIK | |
| Antibody | VH | EVQLVESGGGLVQPGGSLRLSCAVSGFSLTTYGVHWVRQAPGKGLEWVAVIW | 399 |
| h#22A4 | | TGGSTNYNSALMSRATISKDDSKSTVYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 385 |
| | | YLYSGVPSRFSGSGSGTDFTLTISSLQPEDFATYYCQQHYSTPFTFGQGTKV | |
| | | EIK | |
| Antibody | VH | EVQLVESGGGLVQPGGSLRLSCAVSGFSLTTYGVHWVRQAPGKGLEWVAVIW | 400 |
| h#22C4 | | TGGSTNYNSALMSRFTISKDDSKSTVYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 385 |
| | | YLYSGVPSRFSGSGSGTDFTLTISSLQPEDFATYYCQQHYSTPFTFGQGTKV | |
| | | EIK | |
| Antibody | VH | EVQLVESGGGLVQPGGSLRLSCAVSGFSLTTYGVHWVRQAPGKGLEWVAVIW | 401 |
| h#22H5 | | TGGSTNYNSALMSRFTISKDNSKNTVYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 385 |
| | | YLYSGVPSRFSGSGSGTDFTLTISSLQPEDFATYYCQQHYSTPFTFGQGTKV | |
| | | EIK | |
| Antibody | VH | EVQLVESGGGLVQPGGSLRLSCAVSGFSLTTYGVHWVRQAPGKGLEWVAVIW | 401 |
| h#22B6 | | TGGSTNYNSALMSRFTISKDNSKNTVYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 387 |
| | | YLYSGVPSRFSGSGSGTDFTLTISSLQPEDLATYYCQQHYSTPFTFGQGTKV | |
| | | EIK | |
| Antibody | VH | EVQLVESGGGLVQPGGSLRLSCAVSGFSLTTYGVHWVRQAPGKGLEWVAVIW | 402 |
| h#22C6 | | TGGSTNYNSALMSRTTISKDDSKNTVYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 385 |
| | | YLYSGVPSRFSGSGSGTDFTLTISSLQPEDFATYYCQQHYSTPFTFGQGTKV | |
| | | EIK | |
| Antibody | VH | EVQLVESGGGLVQPGGSLRLSCAVSGFSLTTYGVHWVRQAPGKGLEWVAVIW | 403 |
| h#22D6 | | TGGSTNYNSALMSRVTISKDNSKNTVYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 385 |
| | | YLYSGVPSRFSGSGSGTDFTLTISSLQPEDFATYYCQQHYSTPFTFGQGTKV | |
| | | EIK | |
| Antibody | VH | EVQLVESGGGLVQPGGSLRLSCAISGFSLTTYGVHWVRQAPGKGLEWVAVIW | 404 |
| h#22B1 | | TGGSTNYNSALMSRTTISKDDSKNTVYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 385 |
| | | YLYSGVPSRFSGSGSGTDFTLTISSLQPEDFATYYCQQHYSTPFTFGQGTKV | |
| | | EIK | |
| Antibody | VH | EVQLVESGGGLVQPGGSLRLSCAISGFSLTTYGVHWVRQAPGKGLEWVAVIW | 405 |
| h#22E1 | | TGGSTNYNSALMSRFTISKDDSKSTAYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 385 |
| | | YLYSGVPSRFSGSGSGTDFTLTISSLQPEDFATYYCQQHYSTPFTFGQGTKV | |
| | | EIK | |
| Antibody | VH | EVQLVESGGGLVQPGGSLRLSCAISGFSLTTYGVHWVRQAPGKGLEWVAVIW | 406 |
| h#22F1 | | TGGSTNYNSALMSRFTISKDDSKNTVYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 385 |
| | | YLYSGVPSRFSGSGSGTDFTLTISSLQPEDFATYYCQQHYSTPFTFGQGTKV | |
| | | EIK | |
| Antibody | VH | EVQLVESGGGLVQPGGSLRLSCAISGFSLTTYGVHWVRQAPGKGLEWVAVIW | 406 |
| h#22F2 | | TGGSTNYNSALMSRFTISKDDSKNTVYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 387 |
| | | YLYSGVPSRFSGSGSGTDFTLTISSLQPEDLATYYCQQHYSTPFTFGQGTKV | |
| | | EIK | |
| Antibody | VH | EVQLVESGGGLVQPGGSLRLSCAISGFSLTTYGVHWVRQAPGKGLEWVAVIW | 407 |
| h#22G1 | | TGGSTNYNSALMSRFTISKDNSKNTVYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 385 |
| | | YLYSGVPSRFSGSGSGTDFTLTISSLQPEDFATYYCQQHYSTPFTFGQGTKV | |
| | | EIK | |
| Antibody | VH | EVQLVESGGGLVQPGGSLRLSCAISGFSLTTYGVHWVRQAPGKGLEWVAVIW | 408 |
| h#22G3 | | TGGSTNYNSALMSRFTISKDNSKNTVYLQMNSLRAEDTAVYYCARGGYGHTM | |
| | | DYWGQGTLVTVSS | |
| | VL | DIQMTQSPSSLSASVGDRVTITCKASQDVSIAVAWYQQKPGKAPKLLIYSAS | 385 |
| | | YLYSGVPSRFSGSGSGTDFTLTISSLQPEDFATYYCQQHYSTPFTFGQGTKV | |
| | | EIK | |
| Antibody | VH | EVQLVESGGGLVQPGGSLRLSCAISGFSLTTYGVHWVRQAPGKGLEWVAVIW | 408 |
| h#22D5 | | TGGSTNYNSALMSRFTISKDNSKNTVYLQMNSLRAEDTAVYYCARGGYGHTM | |
| | | DYWGQGTLVTVSS | |
| | VL | DIQMTQSPSSLSASVGDRVTITCKASQDVSIAVAWYQQKPGKAPKLLIYSAS | 387 |
| | | YLYSGVPSRFSGSGSGTDFTLTISSLQPEDLATYYCQQHYSTPFTFGQGTKV | |
| | | EIK | |
| Antibody | VH | EVQLVESGGGLVQPGGSLRLSCAISGFSLTTYGVHWVRQAPGKGLEWVAVIW | 409 |
| h#22A3 | | TGGSTNYNSALMSRVTISKDNSKNTVYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 385 |
| | | YLYSGVPSRFSGSGSGTDFTLTISSLQPEDFATYYCQQHYSTPFTFGQGTKV | |
| | | EIK | |
| Antibody | VH | EVQLVESGGGLVQPGGSLRLSCAISGFSLTTYGVHWVRQAPGKGLEWVAVIW | 410 |
| h#22F4 | | TGGSTNYNSALMSRVTISKDDSKNTVYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 387 |
| | | YLYSGVPSRFSGSGSGTDFTLTISSLQPEDLATYYCQQHYSTPFTFGQGTKV | |
| | | EIK | |
| Antibody | VH | EVQLVESGGGLVQPGGSLRLSCAISGFSLTTYGVHWVRQAPGKGLEWVAVIW | 411 |
| h#22E5 | | TGGSTNYNSALMSRFTISKDNSKNTAYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 385 |
| | | YLYSGVPSRFSGSGSGTDFTLTISSLQPEDFATYYCQQHYSTPFTFGQGTKV | |
| | | EIK | |
| Antibody | VH | EVQLVESGGGLVQPGGSLRLSCAISGFSLTTYGVHWVRQAPGKGLEWVAVIW | 412 |
| h#22F6 | | TGGSTNYNSALMSRTTISKDNSKNTVYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 385 |
| | | YLYSGVPSRFSGSGSGTDFTLTISSLQPEDFATYYCQQHYSTPFTFGQGTKV | |
| | | EIK | |
| Antibody | VH | EVQLVESGGGLVQPGGSLRLSCAISGFSLTTYGVHWVRQAPGKGLEWVAVIW | 413 |
| h#22G6 | | TGGSTNYNSALMSRFTISKDDSKNTAYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 385 |
| | | YLYSGVPSRFSGSGSGTDFTLTISSLQPEDFATYYCQQHYSTPFTFGQGTKV | |
| | | EIK | |
| Antibody | VH | EVQLVESGGGLVQPGGSLRLSCAVSGFSLTTYGVHWIRQAPGKGLEWVAVIW | 414 |
| h#22C2 | | TGGSTNYNSALMSRTTISKDDSKNTVYLQMNSLRAEDTAVYYCAKGGYGHTM | |
| | | DYWGQGTLVTVSS | |
| | VL | DIQMTQSPSSLSASVGDRVTITCKASQDVSIAVAWYQQKPGKAPKLLIYSAS | 387 |
| | | YLYSGVPSRFSGSGSGTDFTLTISSLQPEDLATYYCQQHYSTPFTFGQGTKV | |
| | | EIK | |

Functional Variants

Also within the scope of the present disclosure are functional variants of any of the exemplary anti-LILRB2 antibodies as disclosed herein. Such functional variants are substantially similar to the exemplary antibody, both structurally and functionally. A functional variant comprises substantially the same V_{H} and V_{L} CDRs as the exemplary antibody. For example, it may comprise only up to 8 (e.g., 8, 7, 6, 5, 4, 3, 2, or 1) amino acid residue variations in the total CDR regions of the antibody and binds the same epitope of LILRB2 with substantially similar affinity (e.g., having a K_{D} value in the same order). In some instances, the functional variants may have the same heavy chain CDR3 as the exemplary antibody, and optionally the same light chain CDR3 as the exemplary antibody. Alternatively or in addition, the functional variants may have the same heavy chain CDR2 as the exemplary antibody. Such an anti-LILRB2 antibody may comprise a V_{H} fragment having CDR amino acid residue variations in only the heavy chain CDR1, the heavy chain CDR2, or both as compared with the V_{H} of the exemplary antibody. In some examples, the anti-LILRB2 antibody may further comprise a V_{L} fragment, which may have the same V_{L} CDR3 as the exemplary antibody. In some examples, the antibody may have the same V_{L} CDR1 or V_{L} CDR₂ as the exemplary antibody. Alternatively, the antibody may comprise a V_{L} fragment having CDR amino acid residue variations in only the light chain CDR1, the light chain CDR2, or both as compared with the V_{L} of the exemplary antibody.

Alternatively or in addition, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-LILRB2 antibody may comprise heavy chain CDRs that are at least 80% (e.g., 85%, 90%, 95%, or 98%) sequence identity, individually or collectively, as compared with the V_{H} CDRs of an exemplary antibody described herein. Alternatively or in addition, the anti-LILRB2 antibody may comprise light chain CDRs that are at least 80% (e.g., 85%, 90%, 95%, or 98%) sequence identity, individually or collectively, as compared with the V_{L} CDRs as an exemplary antibody described herein. As used herein, “individually” means that one CDR of an antibody shares the indicated sequence identity relative to the corresponding CDR of the exemplary antibody. “Collectively” means that three V_{H} or V_{L} CDRs of an antibody in combination share the indicated sequence identity relative the corresponding three V_{H} or V_{L} CDRs of the exemplary antibody in combination.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the heavy chain of any of the anti-LILRB2 antibodies as described herein may further comprise a heavy chain constant region (CH) or a portion thereof (e.g., CH1, CH2, CH3, or a combination thereof). The heavy chain constant region can of any suitable origin, e.g., human, mouse, rat, or rabbit. Alternatively or in addition, the light chain of the anti-LILRB2 antibody may further comprise a light chain constant region (CL), which can be any CL known in the art. In some examples, the CL is a kappa light chain. In other examples, the CL is a lambda light chain. Antibody heavy and light chain constant regions are well known in the art, e.g., those provided in the IMGT database (www.imgt.org) or at www.vbase2.org/vbstat.php., both of which are incorporated by reference herein.

In some instances, the anti-LILRB2 antibody disclosed herein is an affinity maturation variant of an exemplary antibody provided in Table 1 above. In some examples, the anti-LILRB2 antibody disclosed herein is an affinity maturation variant of antibody 11B12. Such an 11B12 variant may comprise the HC CDR1-CDR3 and LC CDR1-CDR3 motifs listed in Table 2 above. In some instances, such an 11B12 variant may further comprise one or more of the HC FR1-FR4 and LC FR1-FR4 motifs also provided in Table 2 above. Alternatively, the 11B12 variant may comprise the same heavy chain and light chain frameworks as those of 11B12 (see Table 1 above).

Also provided herein are functional variants of h#2E1 (the humanized version of clone 11B12) derived from affinity maturation. Exemplary h#2E1 affinity maturation variants are provided in Table 2 above, each of which is within the scope of the present disclosure. In one specific example, the anti-LILRB2 antibody provided herein is 2E1_FC11.

Such functional variants share a high sequence homology with respect to the heavy chain and/or light chain CDR regions as those of the parent clone h#2E1. For example, a functional variant may comprise up to five amino acid variations in heavy chain CDRs as relative to the heavy chain CDRs of the parent clone (collectively). Alternatively or in addition, the functional variant may comprise up to five amino acid variations in light chain CDRs as relative to the heavy chain CDRs of the parent clone (collectively).

In some examples, the anti-LILRB2 antibody is an affinity maturation variant of h#2E1, for example, clone 2E1_FC11 or those having substantially similar bioactivities as 2E1_FC11. Such anti-LILRB2 antibodies exhibited superior binding affinities to human LILRB2 across a range of binding assays, including ELISA, BLI, SPR, and in-cell binding assays. Further, they exhibited parallel abilities in blocking the interactions of LILRB2 with its major ligands, such as HLA-G and HLA-A2. When compared with known antibodies such as MK4830, JTX₈₀₆₄, and NGM707, 2E1_FC was found to outperform these known antibodies in terms of its functional activities, for example, in the polarization of M2 macrophages towards the pro-inflammatory M1 phase, as observed in the PBMC/LPS model and HMDM/LPS models across multiple healthy human donors.

The anti-LILRB2 antibody 2E1_FC11 or variants thereof may comprise a heavy chain variable region (V_{H}), which comprises (ai) a heavy chain CDR1 comprising GX₁SITSGYX₂WX₃ (SEQ ID NO: 192), in which X₁ is Y or G, X₂ is Y, S, A, or H, and X₃ is N, S, or W; (aii) a heavy chain CDR2 comprising X₄ISYDGNX₅X₆(SEQ ID NO: 194), in which X₄ is S, T, or Y, X₅ is A, I, K, L, M, N, P, S, T, V, or W and X₆ is H, L, N, S, or W; (aiii) a heavy chain CDR3 comprising EEX₇TMX₈TTX₉WFX₁₀Y (SEQ ID NO: 196), in which X₇ is A or S, X₈ is F or I, X₉ is A or G, and X₁₀ is A, G, Q or S. In some examples, the heavy chain CDR2 may comprise X₄ISYDGNX₅X₆YNPSLKN (SEQ ID NO: 416), each of the X variables is as defined herein. In addition, the anti-LILRB2 antibody 2E1_FC11 or variants thereof may comprise (b) a light chain variable region (V_{L}), which comprises (bi) a light chain CDR1 comprising X₁₁X₁₂SEX₁₃IX₁₄SNX₁₅A (SEQ ID NO: 199), in which X₁₁ is E, G, L, N, Q, R, S, T or V, X₁₂ is A or G, X₁₃ is K, N, T, or V, X₁₄ is F or Y, and X₁₅ is L, N, or Q; (bii) a light chain CDR2 comprising GATX₁₆X₁₇X₁₈X₁₉ (SEQ ID NO: 201), in which X₁₆ is E, N, W, or Y, X₁₇ is F, H, L, P, or R, X₁₈ is A, N, R, or S, and X₁₉ is A, G, K, L, R, S, or V; and (biii) a light chain CDR3 comprising QX₂₀FWDX₂₁PYX₂₂ (SEQ ID NO: 203), in which X₂₀ is H or Q, X₂₁ is L, S, T, or Y, and X₂₂ is I, M, R, T, Y, or V.

In some embodiments, the X variables in the heavy chain CDRs may be identical to one or more of those in clone 2E1_FC11: X₁ is Y, X₂ is Y, X₃ is N, X₄ is Y, X₅ is I, X₆ is N, X₇ is S, X₈ is I, X₉ is A, and/or X₁₀ is A. Alternatively or in addition, the X variables in the light chain CDRs may be identical to one or more of those of 2E1_FC11: X₁₁ is R, X₁₂ is A, X₁₃ is N, X₁₄ is Y, X₁₅ is L, X₁₆ is N, X₁₇ is L, X₁₈ is A, X₁₉ is S, X₂₀ is H, X₂₁ is Y, X₂₂ is T, or a combination thereof.

The anti-LILRB2 antibody 2E1_FC11 or variants thereof may comprise the heavy chain and light chain CDRs as described above and human framework regions as also disclosed herein, for example, the heavy and light chain framework regions provided in Table 2 above. In some instances, the anti-LILRB2 antibody 2E1_FC11 or variants thereof may comprise a V_{H} at least 80% (e.g., at least 80%, at least 90%, at least 95%, or above) identical to the V_{H} of 2E1_FC11 (SEQ ID NO: 244); and/or a V_{L} at least 80% (e.g., at least 80%, at least 90%, at least 95%, or above) identical to the V_{L} of 2E1_FC11 (SEQ ID NO: 248).

### II. Methods for Producing Anti-LILRB2 Antibodies

Any of the anti-LILRB2 antibodies described herein can be made by any method known in the art. See, for example, Harlow and Lane, (1998) Antibodies: A Laboratory Manual, Cold Spring Harbor Laboratory, New York. In some embodiments, the anti-LILRB2 antibody may be produced by the conventional hybridoma technology. Alternatively, the anti-LILRB2 antibody may be identified from a suitable library (e.g., a human antibody library). In some instances, high affinity fully human LILRB2 binders may be obtained from a human antibody library, for example, affinity maturation libraries (e.g., having variations in one or more of the CDR regions). See also Examples below. There are a number of routine methods known in the art to identify and isolate antibodies capable of binding to the target antigens described herein, including phage display, yeast display, ribosomal display, or mammalian display technology.

If desired, an antibody (monoclonal or polyclonal) of interest (e.g., produced by a hybridoma cell line or isolated from an antibody library) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to, e.g., humanize the antibody or to improve the affinity (affinity maturation), or other characteristics of the antibody. For example, the constant region may be engineered to more resemble human constant regions to avoid immune response if the antibody is from a non-human source and is to be used in clinical trials and treatments in humans. Alternatively or in addition, it may be desirable to genetically manipulate the antibody sequence to obtain greater affinity and/or specificity to the target antigen and greater efficacy in enhancing the activity of LILRB2. It will be apparent to one of skill in the art that one or more polynucleotide changes can be made to the antibody and still maintain its binding specificity to the target antigen.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions of V_{H} and V_{L} of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human V_{H} and V_{L} chains having amino acid sequences that are homologous to those of the parent non-human antibody are identified from any antibody gene database using the parent V_{H} and V_{L} sequences as search queries. Human V_{H} and V_{L} acceptor genes are then selected. The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage-display, yeast-display, mammalian cell-display, or mRNA-display scFv library and scFv clones specific to LILRB2 can be identified from the library following routine procedures.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence, to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries).

Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of LILRB2 have been replaced (swapped) with sequences from a closely related, but antigenically distinct protein (such as another member of the tumor necrosis factor receptor family). By assessing binding of the antibody to the mutant LILRB2, the importance of the particular antigen fragment to antibody binding can be assessed.

Alternatively, competition assays can be performed using other antibodies known to bind to the same antigen to determine whether an antibody binds to the same epitope as the other antibodies. Competition assays are well known to those of skill in the art. In some examples, an anti-LILRB2 antibody or a bispecific antibody as disclosed herein can be prepared by recombinant technology as exemplified below.

Nucleic acids encoding the heavy and light chain of an anti-LILRB2 antibody or a bispecific antibody as described herein can be cloned into one expression vector, each nucleotide sequence being in operable linkage to a suitable promoter. In one example, each of the nucleotide sequences encoding the heavy chain and light chain is in operable linkage to a distinct prompter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987); Gossen and Bujard (1992); M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an anti-LILRB2 antibody or encodes both chains of a two-chain bispecific antibody as also described herein. The recombinant expression vector can be introduced into a suitable host cell (e.g., a dhfr-CHO cell) by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the anti-LILRB2 antibody or one of the two chains of a two-chain bispecific antibody disclosed herein and the other encoding the light chain of the anti-LILRB2 antibody or the other chain of the bispecific antibody. Both of the two recombinant expression vectors can be introduced into a suitable host cell (e.g., dhfr-CHO cell) by a conventional method, e.g., calcium phosphate-mediated transfection. Alternatively, each of the expression vectors can be introduced into a suitable host cell. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-LILRB2 antibody or any of the bispecific antibodies as described herein, vectors (e.g., expression vectors) containing such; and host cells comprising the vectors are within the scope of the present disclosure.

### III. Uses of Anti-LILRB2 Antibodies

Any of the anti-LILRB2 antibodies disclosed herein can be used for therapeutic, diagnostic, and/or research purposes, all of which are within the scope of the present disclosure.

### Pharmaceutical Compositions

The antibodies, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The antibodies, or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation. For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing an antibody with Intralipid™ or the components thereof (soybean oil, egg phospholipids, glycerol and water). Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

Therapeutic Applications

To practice the method disclosed herein, an effective amount of the pharmaceutical composition described herein, comprising any of the anti-LILRB2 antibodies can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes.

The subject to be treated by the methods described herein can be a mammal, more preferably a human or a non-human primate. Mammals include, but are not limited to, farm animals, sport animals, pets, primates, horses, dogs, cats, mice and rats. A human subject who needs the treatment may be a human patient having, at risk for, or suspected of having a target disease/disorder (a cancer such as a solid tumor), which would benefit from modulating immune responses mediated by LILRB2⁺ immune cells such as macrophages, e.g., enhancing anti-tumor immune responses and/or reducing tumor cell immune evasion. Examples of such target cancers include, gastric cancer, esophageal cancer, pancreatic cancer, non-small cell lung cancer, ovarian cancer, colon cancer, hepatic cancer, head-neck cancer, and cancer of the gallbladder.

A subject having a target cancer can be identified by routine medical examination, e.g., laboratory tests, organ functional tests, CT scans, or ultrasounds. In some embodiments, the subject to be treated by the method described herein may be a human cancer patient who has undergone or is subjecting to an anti-cancer therapy, for example, chemotherapy, radiotherapy, immunotherapy, or surgery.

A subject suspected of having any of such target disease/disorder might show one or more symptoms of the disease/disorder. A subject at risk for the disease/disorder can be a subject having one or more of the risk factors for that disease/disorder.

As used herein, “an effective amount” refers to the amount of each active agent required to confer therapeutic effect on the subject, either alone or in combination with one or more other active agents. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein may be determined empirically in individuals who have been given one or more administration(s) of the antibody. Individuals are given incremental dosages of the agonist. To assess efficacy of the agonist, an indicator of the disease/disorder can be followed.

Generally, for administration of any of the antibodies described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of an antibody as described herein will depend on the specific antibody, antibodies, and/or non-antibody peptide (or compositions thereof) employed, the type and severity of the disease/disorder, whether the antibody is administered for preventive or therapeutic purposes, previous therapy, the patient's clinical history and response to the agonist, and the discretion of the attending physician. Typically, the clinician will administer an antibody, until a dosage is reached that achieves the desired result. In some embodiments, the desired result is an increase in anti-tumor immune response in the tumor microenvironment. Methods of determining whether a dosage resulted in the desired result would be evident to one of skill in the art. Administration of one or more antibodies can be continuous or intermittent, depending, for example, upon the recipient's physiological condition, whether the purpose of the administration is therapeutic or prophylactic, and other factors known to skilled practitioners. The administration of an antibody may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, any of the antibodies disclosed herein may be administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Targeted delivery of therapeutic compositions containing one or more nucleic acids such as expression vectors for producing any of the anti-LILRB2 antibodies or bispecific antibodies can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

Therapeutic compositions containing a polynucleotide (e.g., those encoding the antibodies described herein) can be administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

The therapeutic polynucleotides described herein can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent No. 0524968. Additional approaches are described in Philip, Mol. Cell. Biol. (1994) 14:2411, and in Woffendin, Proc. Natl. Acad. Sci. (1994) 91:1581.

The particular dosage regimen, i.e., dose, timing and repetition, used in the method described herein will depend on the particular subject and that subject's medical history.

In some embodiments, more than one antibody, or a combination of an antibody and another suitable therapeutic agent, may be administered to a subject in need of the treatment. The antibody can also be used in conjunction with other agents that serve to enhance and/or complement the effectiveness of the agents. Treatment efficacy for a target disease/disorder such as those disclosed herein can be assessed by methods well-known in the art.

In some embodiments, any of the anti-LILRB2 disclosed herein may be co-used with another anti-cancer agent, for example, a chemotherapeutic agent, an immunotherapeutic agent, or a combination thereof. As used herein, the term “combination,” “combined,” and related terms refers to the simultaneous or sequential administration of multiple therapeutic agents in accordance with this disclosure. For example, any of the anti-LILRB2 antibodies or any of the bispecific antibodies as disclosed herein may be administered with another therapeutic agent simultaneously or sequentially in separate unit dosage forms or together in a single unit dosage form.

Diagnostic Applications

Any of the anti-LILRB2 antibodies disclosed here may be used for detecting and quantifying LILRB2 protein levels in a biological sample using a conventional method, for example, any immunohistological method known to those of skill in the art (see, e.g., Jalkanen, et al., J. Cell. Biol. 101:976-985 (1985); Jalkanen et al., J. Cell Biol. 105:3087-3096 (1987)). Other antibody-based methods useful for detecting LILRB2 protein expression include immunoassays, such as the enzyme linked immunosorbent assay (ELISA), immunoprecipitation, or Western blotting. Suitable assays are described in more detail elsewhere herein.

The term “biological sample” means any biological sample obtained from an individual, cell line, tissue culture, or other source of cells potentially expressing LILRB2. Methods for obtaining tissue biopsies and body fluids from mammals are well known in the art.

To perform the method disclosed herein, any of the anti-LILRB2 antibodies as disclosed herein can be brought in contact with a sample suspected of containing a target antigen as disclosed herein, for example, a human LILRB2 protein or a LILRB2⁺ cell. In general, the term “contacting” or “in contact” refers to an exposure of the anti-LILRB2 antibody disclosed herein with the sample suspected of containing the target antigen for a suitable period under suitable conditions sufficient for the formation of a complex between the anti-LILRB2 antibody and the target antigen in the sample, if any. The antibody-antigen complex thus formed, if any, can be determined via a routine approach. Detection of such an antibody-antigen complex after the incubation is indicative of the presence of the target antigen in the sample. When needed, the amount of the antibody-antigen complex can be quantified, which is indicative of the level of the target antigen in the sample.

In some examples, the anti-LILRB2 antibodies as described herein can be conjugated to a detectable label, which can be any agent capable of releasing a detectable signal directly or indirectly. The presence of such a detectable signal or intensity of the signal is indicative of presence or quantity of the target antigen in the sample. Alternatively, a secondary antibody specific to the anti-LILRB2 antibody or specific to the target antigen may be used in the methods disclosed herein. For example, when the anti-LILRB2 antibody used in the method is a full-length antibody, the secondary antibody may bind to the constant region of the anti-LILRB2 antibody. In other instances, the secondary antibody may bind to an epitope of the target antigen that is different from the binding epitope of the anti-LILRB2 antibody. Any of the secondary antibodies disclosed herein may be conjugated to a detectable label.

Any suitable detectable label known in the art can be used in the assay methods described herein. In some embodiments, a detectable label can be a label that directly releases a detectable signal. Examples include a fluorescent label or a dye. A fluorescent label comprises a fluorophore, which is a fluorescent chemical compound that can re-emit light upon light excitation. Examples of fluorescent label include, but are not limited to, xanthene derivatives (e.g., fluorescein, rhodamine, Oregon green, eosin, and Texas red), cyanine derivatives (e.g., cyanine, indocarbocyanine, oxacarbocyanine, thiacarbocyanine, and merocyanine), squaraine derivatives and ring-substituted squaraines (e.g., Seta and Square dyes), squaraine rotaxane derivatives such as SeTau dyes, naphthalene derivatives (e.g., dansyl and prodan derivatives), coumarin derivatives, oxadiazole derivatives (e.g., pyridyloxazole, nitrobenzoxadiazole and benzoxadiazole), anthracene derivatives (e.g., anthraquinones, including DRAQ5, DRAQ7 and CyTRAK Orange), pyrene derivatives such as cascade blue, oxazine derivatives (e.g., Nile red, Nile blue, cresyl violet, and oxazine 170), acridine derivatives (e.g., proflavin, acridine orange, and acridine yellow), arylmethine derivatives (e.g., auramine, crystal violet, and malachite green), and tetrapyrrole derivatives (e.g., porphin, phthalocyanine, and bilirubin). A dye can be a molecule comprising a chromophore, which is responsible for the color of the dye. In some examples, the detectable label can be fluorescein isothiocyanate (FITC), phycoerythrin (PE), biotin, Allophycocyanin (APC) or Alexa Fluor® 488.

In some embodiments, the detectable label may be a molecule that releases a detectable signal indirectly, for example, via conversion of a reagent to a product that directly releases the detectable signal. In some examples, such a detectable label may be an enzyme (e.g., β-galactosidase, HRP or AP) capable of producing a colored product from a colorless substrate.

### IV. Kits Comprising Anti-LILRB2 Antibodies and Uses Thereof

The present disclosure also provides kits comprising any of the anti-LILRB2 antibodies disclosed herein. Such kits can be used for any of the applications of such antibodies as disclosed herein, for example, for use in treating or alleviating a target disease, such as a cancer as disclosed herein, or for detecting presence or measuring the amount of LILRB2 protein or LILRB2⁺ cells in a biological sample. Such kits can include one or more containers comprising an anti-LILRB2 antibody or a bispecific antibody as those described herein.

In some embodiments, the kit can comprise instructions for use in accordance with any of the methods described herein. The included instructions can comprise a description of administration of the anti-LILRB2 antibody or the bispecific antibody to treat, delay the onset, or alleviate a target disease as those described herein. The kit may further comprise a description of selecting an individual suitable for treatment based on identifying whether that individual has the target disease. In still other embodiments, the instructions comprise a description of administering an antibody to an individual at risk of the target disease.

The instructions relating to the use of the anti-LILRB2 antibody generally include information as to dosage, dosing schedule, and route of administration for the intended treatment. The containers may be unit doses, bulk packages (e.g., multi-dose packages) or sub-unit doses. Instructions supplied in the kits of the present disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease, such as cancer or immune disorders (e.g., an autoimmune disease). Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-LILRB2 antibody or a bispecific antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Generation and Characterization of Mouse Anti-LILRB2 Antibodies

This example illustrates generation of mouse anti-LILRB2 antibodies via conventional hybridoma technology and characterization of exemplary clones for binding specificities and affinities to target antigens.

A. Generation of Anti-LILRB2 Antibodies

Plasmid encoding full length human LILRB2 was mixed with in vivo-jetPEI™-Gal (Polyplus-transfection, Cat #202-10G) and injected into female BALB/c mice intravenously through tail vein followed the instructions. A total of 3 injections were given within a 3-week interval. Ten days after the final DNA injection, additional 40 μg plasmid was injected intravenously with the same method above and 5 million of 293T cells overexpressing human LILRB2 (HEK293T-LILRB2) were also intraperitoneal injected for boosting. Three days later, the mouse was euthanized and the spleen cells were harvested for fusion.

To produce monoclonal hybridomas, mouse myeloma cell Sp2/0 were grown to a logarithmic growth phase and fused with the immunized mouse spleen cells at a ratio of 1:2 or 1:3 in the presence of polyethylene glycol/Dimethyl sulfoxide (PEG/DMSO; 45%/5%) solution (Hybri-max, Sigma, P7181, D2650). The cells were seeded in 96 well plate and cultured selectively with hypoxanthine-aminopterin-thymidine (HAT) (Sigma, H0262) medium for 5-7 days. Then the HAT medium was replaced with HT medium and continually cultured for 5-7 days. When there are hybridoma cell clones in the 96 well plate, the culture supernatant was used for cell ELISA with HEK293T-LILRB2 and HEK293T concurrently. The clone which reacted with HEK293T-LILRB2 only was subcloned conventionally. After 3 to 5 rounds of subcloning, until the all of the single clones were positive with HEK293T-LILRB2 only, selected a clone to establish the hybridoma cell line.

To produce monoclonal antibody for characterization, the selected monoclonal hybridoma cells were injected into the peritoneal cavity of Balb/C mice to produce monoclonal antibodies in the ascitic fluid. The monoclonal antibody was purified from the ascitic fluid with protein G for further characterization.

B. Characterization of Exemplary Anti-LILRB2 Antibodies

(i) Binding Specificities Across LILRB1, LILRB2, and LILRB3 Via ELISA

2×10⁴ 293T cells, 293T-LILRB1 cells, 293T-LILRB2 cells, or 293T-LILRB3 cells were placed in each well in a 96-well plate and cultured for 18 hours. The cells were then immobilized with glutaraldehyde in the presence of 5% defatted milk at room temperature for 2 hours. An anti-LILRB2 antibody (2 μg/ml) was added to each well and incubated at room temperature for 2 hours. An HRP-labelled goat anti-mouse secondary antibody was then added into each well and incubated at room temperature for one hour. An HRP substrate, o-phenylenediamine dihydrochloride (OPD) was then added to each well. The plate was incubated at room temperature for 15 min to allow color development and OD₄₉₀ values were determined.

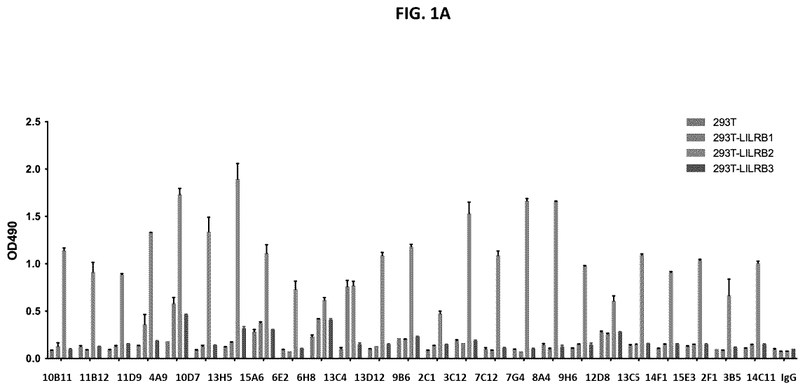

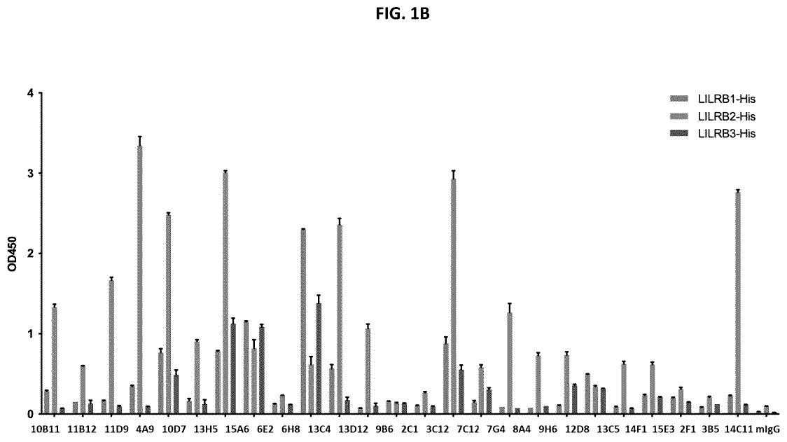

As shown in FIG. 1A, most of the clones showed high binding specificities to LILRB2 relative to LILRB1 and/or LILRB3 (e.g., for clone 9B6, the OD490s of LILRB2, LILRB1, and LILRB3 binding are 1.426, 0.1215, and 0.1255, respectively; For clone 14F1, the OD490s of LILRB2, LILRB1, and LILRB3 binding are 1.2115, 0.1715, and 0.1725, respectively; for clone 9H6, the OD490 values of LILRB2, LILRB1, and LILRB3 binding are 1.8625, 0.177, and 0.139, respectively), and clones such as 13C4 showed some cross-reactivities across the LILRB antigens.

The binding specificities were also determined using recombinant LILRB1, LILRB2, and LILRB3 proteins in an ELISA assay. Similar results were observed as shown in FIG. 1B.

(ii) Binding Specificities Across Human and Non-Human Primate LILRB2 Via Flow Cytometry

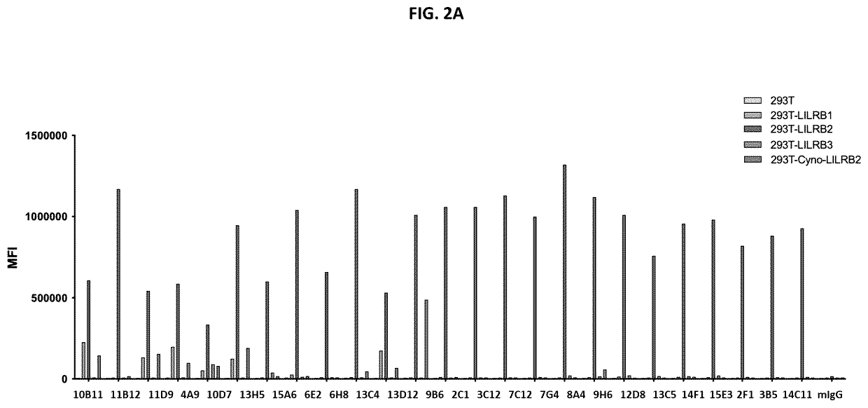

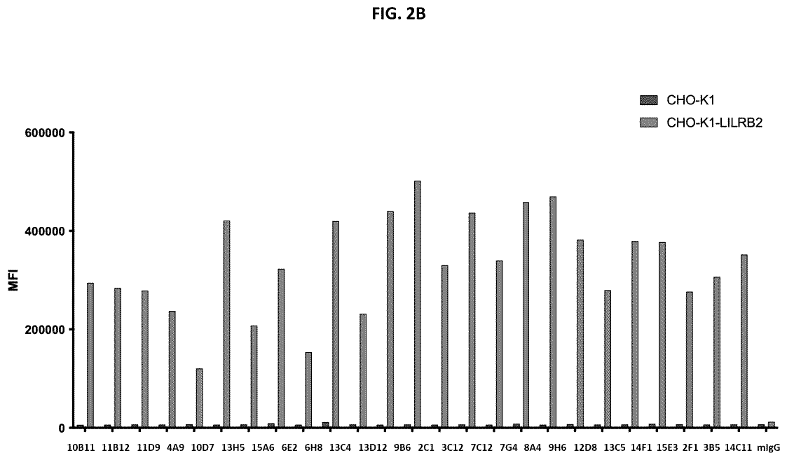

2×10⁵ 293T cells, 293T-LILRB1 cells, 293T-LILRB2 cells, 293T-LILRB3, or 293T-cyno-LILRB2 cells were placed in each well in a 96-well plate and cultured for 18 hours. The cells were then immobilized with glutaraldehyde in the presence of 5% defatted milk at room temperature for 2 hours. An anti-LILRB2 antibody (5 μg/ml) was added to each well and incubated at room temperature for 1 hours. An Alex Flour®-labelled goat anti-mouse secondary antibody (1:2,000 dilution) was then added into each well and incubated at room temperature for 30′. The cells were washed twice with PBS after the staining. Fluorescence intensities (MFI) were measured. The results are shown in FIG. 2A. Certain clones (e.g., 9B6 and 9H6) showed high binding specificities to LILRB2 and cross-reacted with both human and cynomolgus monkey LILRB2 antigens.

(iii) Binding Activity by Flow Cytometry

2×10⁵ CHO-K1 cells and CHO-KI cells expressing human LILRB2 (CHO-K1-LILRB2) cells were placed in each well in a 96-well plate and cultured for 18 hours. The cells were then immobilized with glutaraldehyde in the presence of 5% defatted milk at room temperature for 2 hours. An anti-LILRB2 antibody (5 μg/ml) was added to each well and incubated at room temperature for 1 hours. An Alex Flour®-labelled goat anti-mouse secondary antibody was then added into each well and incubated at room temperature for 30′.

The cells were then analyzed by flow cytometry. The results are shown in FIG. 2B. All tested clones showed no binding to the parent CHO-K1 cells and all showed high binding activities to the CHO-K1-LILRB2 cells.

(iv) Binding Affinities of Exemplary Anti-LILRB2 Antibodies Via ELISA

Anti-LILRB2 antibodies at different concentrations were used in ELISA assays as described above. The binding affinities were determined following conventional methods and are provided in Table 5 below.

TABLE 5
Binding Affinities of Exemplary Anti0LILRB2 Antibodies

| Clones | Kd (M) | Clones | Kd (M) |
|---|---|---|---|
| 10B11 | 1.95E−10 | 3C12 | 5.01E−09 |
| 11B12 | 3.20E−09 | 7C12 | 6.31E−10 |
| 11D9 | 1.43E−09 | 7G4 | 1.53E−09 |
| 4A9 | 2.16E−10 | 8A4 | 4.93E−10 |
| 10D7 | 7.05E−10 | 9H6 | 1.04E−09 |
| 13H5 | 1.39E−09 | 12D8 | 4.37E−09 |
| 15A6 | 7.25E−10 | 13C5 | 3.14E−10 |
| 6E2 | 7.25E−10 | 14F1 | 1.24E−09 |
| 6H8 | 5.70E−09 | 15E3 | 4.06E−10 |
| 13C4 | 1.17E−08 | 2F1 | 2.18E−09 |
| 13D12 | 2.48E−09 | 3B5 | 2.93E−09 |
| 9B6 | 9.43E−10 | 14C11 | 8.04E−10 |
| 2C1 | 4.74E−10 | | |

(v) Binding Affinities of Exemplary Anti-LILRB2 Antibodies Via SPR

Surface plasmon resonance (SPR) was used to examine binding activities of exemplary anti-LILBR2 antibodies disclosed herein. Briefly, the anti-LILBR2 antibody was diluted in a coating buffer to a concentration of 30 μg/ml, loaded onto CM5 chips at 10 μl/min to allow coating of the antibody on the surface of the CM5 chip. His-tagged human LILBR2 polypeptide was diluted in a loading buffer to produces samples with antigen concentrations of 125-0.244 nM. Binding affinity was measured based on the 1:1 binding model. The results are shown in Table 6 below.

TABLE 6
Anti-LILBR2 Antibody Binding Activity Determined by SPR

| | Capture | Analyte | | | | | |
|---|---|---|---|---|---|---|---|
| | Level | Conc. | | | | Rmax | Chi² |
| Antibody | (RU) | (nM) | ka (1/Ms) | kd (1/s) | KD (M) | (RU) | (RU²) |
| 11B12 | 158.9 | 0.244-31.25 | 9.05E+05 | 3.20E−04 | 3.53E−10 | 73.5 | 0.35 |
| 10D7 | 156.7 | 0.977-125 | 3.38E+05 | 1.07E−04 | 3.17E−10 | 68.7 | 0.91 |
| 15A6 | 170.4 | 0.977-125 | 2.51E+05 | 1.37E−03 | 5.45E−09 | 41.1 | 0.29 |
| 9B6 | 153.2 | 0.488-62.5 | 8.09E+05 | 8.64E−04 | 1.07E−09 | 70.8 | 1.5 |
| 7C12 | 167.1 | 0.977-125 | 2.63E+05 | 2.09E−04 | 7.92E−10 | 65.9 | 1.17 |
| 7G4 | 155.4 | 0.488-62.5 | 4.32E+05 | 6.77E−04 | 1.57E−09 | 76.9 | 1.35 |
| 8A4 | 162.5 | 0.244-31.25 | 9.40E+05 | 1.74E−04 | 1.85E−10 | 76 | 0.28 |
| 9H6 | 168.9 | 0.977-125 | 1.83E+05 | 4.72E−06 | 2.57E−11 | 76.1 | 0.22 |
| 12D8 | 178.2 | 0.488-62.5 | 6.18E+05 | 1.40E−04 | 2.27E−10 | 86.9 | 1.08 |
| 14C11 | 177.4 | 0.977-125 | 2.60E+05 | 2.50E−04 | 9.62E−10 | 72.1 | 1.01 |

(vi) Exemplary Anti-LILBR2 Antibodies Block HLA-A2 Binding in CHO-K1-huLILRB2 Cells

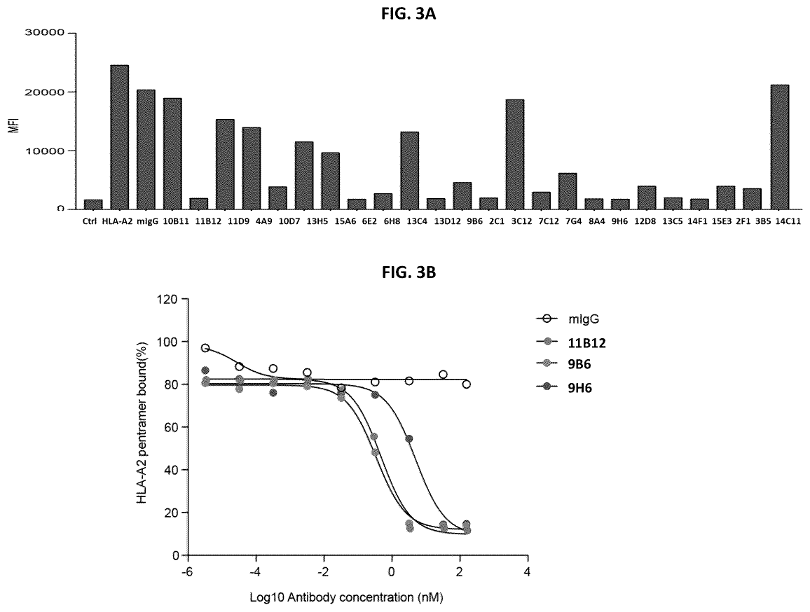

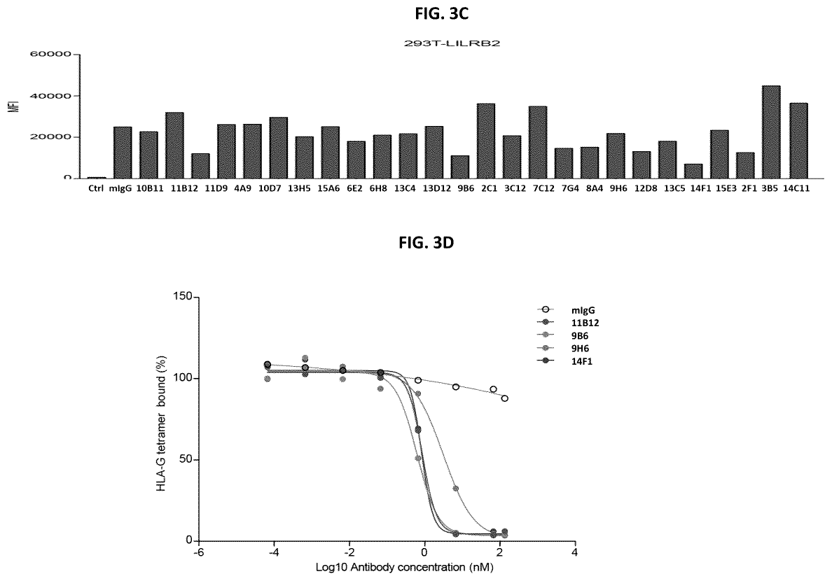

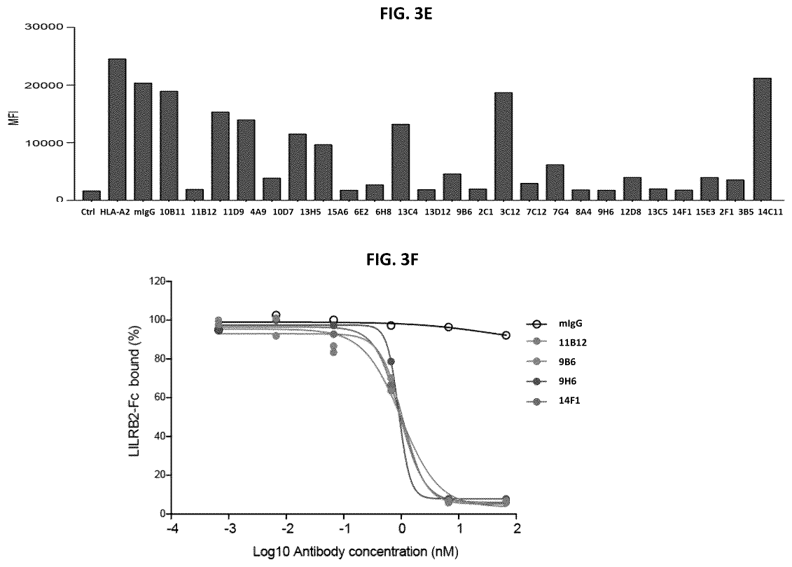

Pre-experimentation was performed to determine parameters for this assay. PE-labelled HLA-A2 tetramer at the concentration of 0.25 μg/ml and an anti-LILRB2 antibody at the concentration of 2.5 μg/ml were incubated with CHO-K1-huLILRB2 cells for 10 minutes. The cells were washed twice and then analysed by flow cytometry. A number of anti-LILBR2 antibodies showed high levels of blockade against HLA-A2 binding to CHO-K1-huLILRB2 cells. FIG. 3A.

Clones 11B12, 9B6, and 9H6 were selected for dose-dependent blockade assays against HLA-A2 from binding to CHO-K1-huLILRB2 cells. A mouse IgG (mIgG) antibody was used as a negative control. The results are shown in FIG. 3B. It is determined that the IC₅₀ values of clones 11B12, 9B6 and 9H6 are 0.475 nM, 0.34 nM and 4.972 nM, respectively.

(vi) Exemplary Anti-LILBR2 Antibodies Block HLA-G Binding in 293T-huLILRB2 and THP1-huLILRB2 Cells

Pre-experimentation was performed to determine parameters for this assay. PE-labelled HLA-G tetramer at the concentration of 0.3 μg/ml and an anti-LILRB2 antibody at the concentration of 0.02 μg/ml or 0.0002 μg/ml were incubated with 293T-huLILBR2 cells or THP1-huLILRB2 cells for 10 minutes. The cells were washed twice and then analysed by flow cytometry. A number of anti-LILBR2 antibodies showed high levels of blockade against HLA-A2 binding to CHO-K1-huLILRB2 cells. FIG. 3C.

Clones 11B12, 9B6, 9H6, and 14F1 were selected for dose-dependent blockade assays against HLA-G from binding to 293T-huLILBR2 cells. A mouse IgG (mIgG) antibody was used as a negative control. The results are shown in FIG. 3D. All tested anti-LILBR2 clones showed dose-dependent blockade against HLA-G tetramer from binding to 293T-huLILBR2 cells. The IC50 values of clone 11B12, 9B6, 9H6, and 14F1 are 0.8566 nM, 0.6181 nM, 3.121 nM, and 0.82 nM, respectively. Clone 9B6 showed the best blockade effect.

(viii) Exemplary Anti-LILBR2 Antibodies Block ANGPTL2 Binding with Human LILRB2

His-tagged ANGPTL2 protein (1 μg/ml) was placed in a 96-well plate (100 μg/well) and incubated at 4° C. overnight. Afterwards, LILRB2-Fc fusion protein at 0.25 mg/ml was incubated with the anti-LILRB2 antibody at various concentrations at room temperature for one hour and then added into the His-ANGPTL2 coated plate. The plate was placed in a shaker at 500 rpm for 2 hours at room temperature. HRP-labelled goat anti-human Fc antibody (1:10,000 dilution) was added to the plate (100 μl/well). After incubation at room temperature for 1 hour, TMB was added for color development. As shown in FIG. 3E, a number of exemplary anti-LILRB2 antibodies tested showed blockade effect against binding of ANGPTL2 to LILRB2.

Clones 11B12, 9B6, 9H6, and 14F1 were selected for dose-dependent blockade assays. A mouse IgG (mIgG) antibody was used as a negative control. The results are shown in FIG. 3F. All tested anti-LILBR2 clones showed dose-dependent blockade against HLA-G tetramer from binding to 293T-huLILBR2 cells. The IC₅₀ values are around 1 nM. The IC50 values of clone 11B12, 9B6, 9H6, and 14F1 are 1.001 nM, 1.007 nM, 0.945 nM, and 0.942 nM, respectively.
(ix) Exemplary Anti-LILBR2 Antibodies Enhanced TNFα Expression and Suppressed IL-10 Expression in LPS-Stimulated PBMCs

Freshly isolated human peripheral blood mononuclear cells (PBMCs) were cultured in RPMI1640 supplemented with 10% fetal bovine serum (FBS). The cells were first treated with exemplary anti0LILRB2 antibody 11B12, 9B6, 9H6, or 14F1 and then stimulated with lipopolysaccharides (LPS) at 100 ng/ml. The supernatant was collected from the cell culture and subjected to an ELISA assay to measure levels of human tumor necrosis factor alpha (TNFα) and IL-10. A mouse IgG isotype antibody was used as a control.

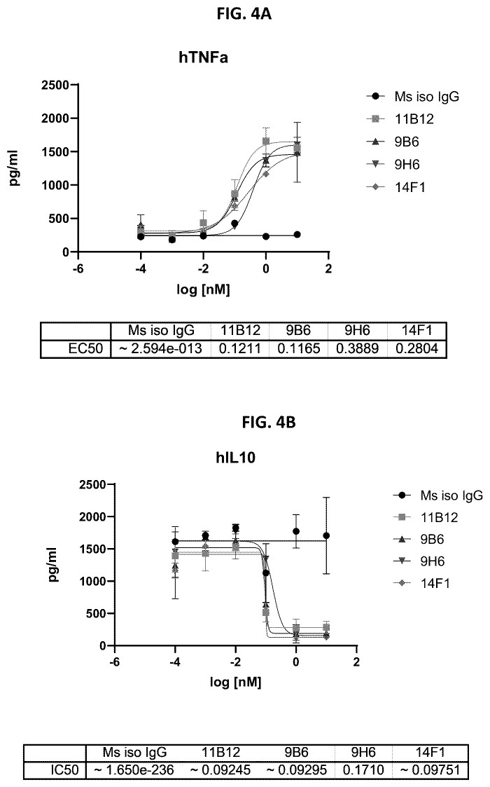

As shown in FIGS. 4A and 4B, the exemplary anti-LILRB2 antibodies enhanced levels of TNFα secreted by the LPS-stimulated PBMCs but suppressed IL-10 expression by the LPS-stimulated PBMCs.

### Example 2: Generation and Characterization of Humanized Anti-LILRB2 Antibodies

This example illustrates generation of humanized anti-LILRB2 antibodies and characterization of their binding and biological activities.

A. Generation of Humanized Antibodies

Clones 11B12, 9B6, and 14F1 were chosen as exemplary parent mouse anti-LILRB2 antibodies for humanization following conventional antibody humanization approaches. See, e.g., disclosures herein. Briefly, the heavy chain and light chain complementarity determining regions of the mouse parent clone were grafted into a suitable human V_{H} and V_{L} acceptor chains. In some instances, back mutations were performed at selected framework positions to revert the amino acid residue in the human acceptor chains to that of the mouse parent clone. Exemplary humanized antibodies of clone 11B12, 9B6, and 14F1 are provided in Tables 2-4 above.

B. Characterization of Humanized Antibodies

(i) Binding Activity to Cell Surface LILRB2

The binding affinities of humanized LILRB2 antibodies to 293T-LILRB2 cells were measured by a flow cytometry assay. Briefly, serial diluted humanized anti-LILRB2 antibodies were incubated with 293T-LILRB2 cells at room temperature for 30 minutes, followed by staining of a secondary APC labeled anti-human IgG antibody. The cells were washed and then subject to flow cytometry to measure levels of fluorescent signals.

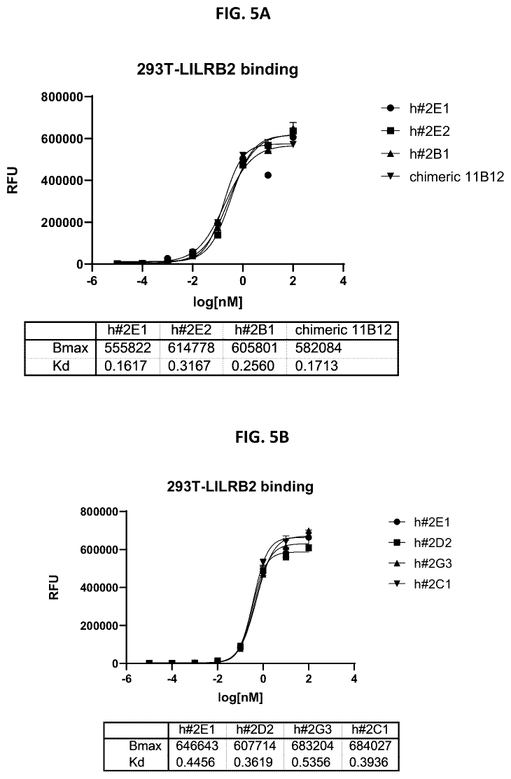

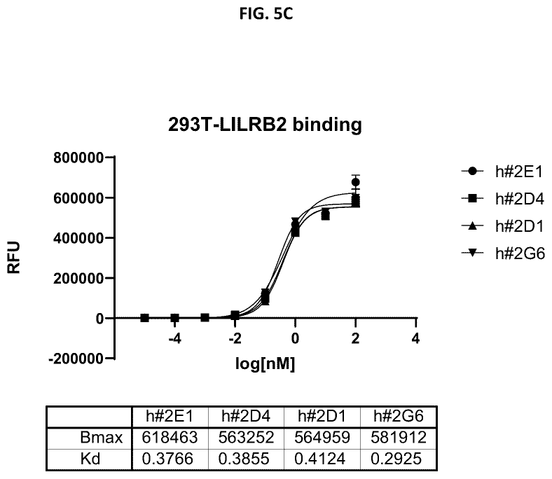

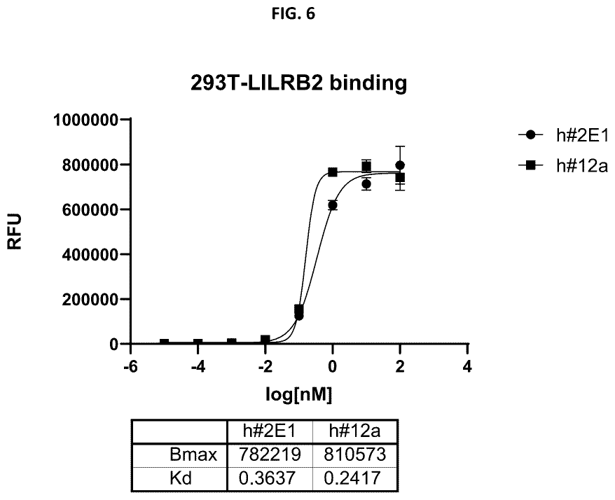

The binding activity of exemplary humanized antibodies derived from parent clone 11B12 is shown in FIGS. 5A-5C. FIG. 6 shows binding activity of exemplary humanized antibodies derived from parent clone 9B6.

(ii) Blockade of HLA-G Binding to 293T-LILRB2 Cells

0.2 μg His-tagged HLA-G tetramer was incubated with 293T-LILRB2 cells at room temperature for 45 minutes to allow for HLA-G/LILRB2 binding. Serial diluted humanized LILRB2 antibodies were then added to the mixture and incubated at room temperature for another 45 minutes. The binding of HLA-G/LILRB2 was detected afterwards using a secondary PE-labeled anti-His tag antibody via flow cytometry.

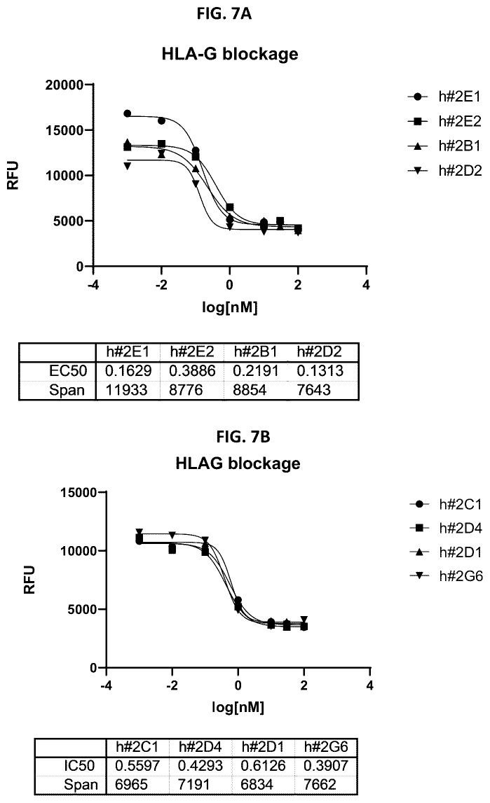

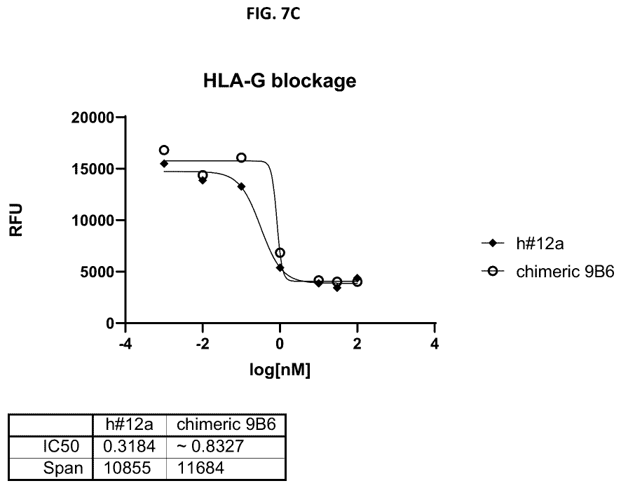

FIGS. 7A and 7B show that exemplary humanized antibodies derived from 11B12 all blocked binding of HLA-G to 293T-LILRB2 cells. FIG. 7C show that exemplary humanized antibodies derived from 9B6 blocked binding of HLA-G to 293T-LILRB2 cells.

(iii) Blockade of HLA-A2 Binding to 293T-LILRB2 Cells

0.2 ug His-tagged HLA-A2 tetramer was incubated with 293T-LILRB2 cells at room temperature for 45 minutes to allow for HLA-G/LILRB2 binding. Serial diluted h#2E1 antibodies were then added to the mixture and incubated at room temperature for another 45 minutes. The binding of HLA-G/LILRB2 was detected afterwards using a secondary PE-labeled anti-His tag antibody via flow cytometry.

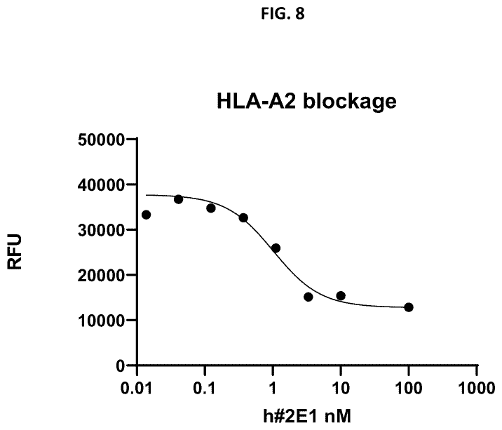

FIG. 8 show that exemplary humanized antibodies derived from 11B12 blocked binding of HLA-A2 to 293T-LILRB2 cells.

### Example 3. Affinity Maturation and Characterization

Humanized antibody h#2E1 (derived from mouse parent 11B12) was selected for affinity maturation. Briefly, an affinity maturation library comprising mutations at certain positions of heavy and light chain CDRs was constructed via conventional methods.

Exemplary matured anti-LILRB2 antibodies were provided in Table 2 above.

(i) Binding Activity

This affinity maturation library was screened against human LILRB2 to identify antibodies having high binding affinity to LILRB2. Briefly, an ELISA plate was coated with 0.1 ug/well recombinant human LILRB2 protein. Serial diluted maturated h#2E1 antibodies were incubated in the LILRB2 coated plate for 2 hrs. The plate was washed and a secondary HRP-labeled anti-human IgG was then added to each well. After being incubated at room temperature for 1 hour, the colorimetric ELISA signals were developed by adding the TMB substrate and reaction was stopped by 1M H3PO4. Table 7 below provides binding activities of exemplary matured antibodies to recombinant LILRB2 protein.

Further, binding of the matured antibodies to 293T-LILRB2 cells was examined using flow cytometry as follows. Serial diluted exemplary matured anti-LILRB2 antibodies were incubated with 293T-LILRB2 cells, followed by incubation with a secondary APC labeled anti-human IgG antibody. The fluorescent signals were detected by flow cytometry. Table 8 below provides binding activities of exemplary matured antibodies to 293T-LILRB2 cells.

(ii) Blockade of HLA-G Binding to 293T-LILRB2 Cells

0.2 μg His-tagged HLA-G tetramer was incubated with 293T-LILRB2 cells at room temperature for 45 minutes to allow for HLA-G/LILRB2 binding. Serial diluted humanized LILRB2 antibodies were then added to the mixture and incubated at room temperature for another 45 minutes. The binding of HLA-G/LILRB2 was detected afterwards using a secondary PE-labeled anti-His tag antibody via flow cytometry.

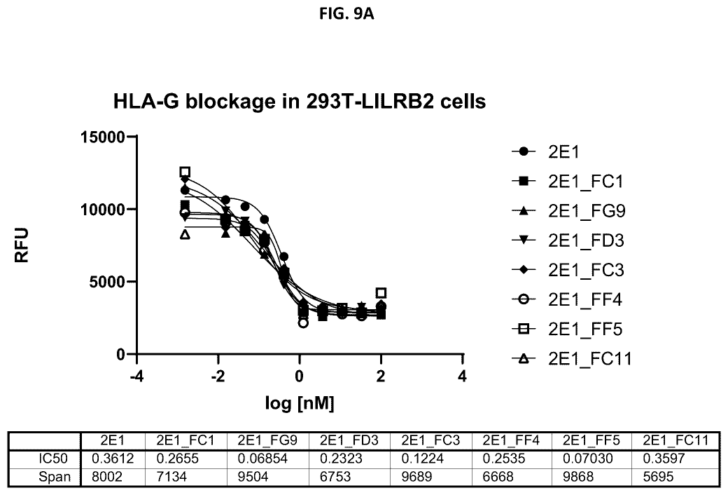

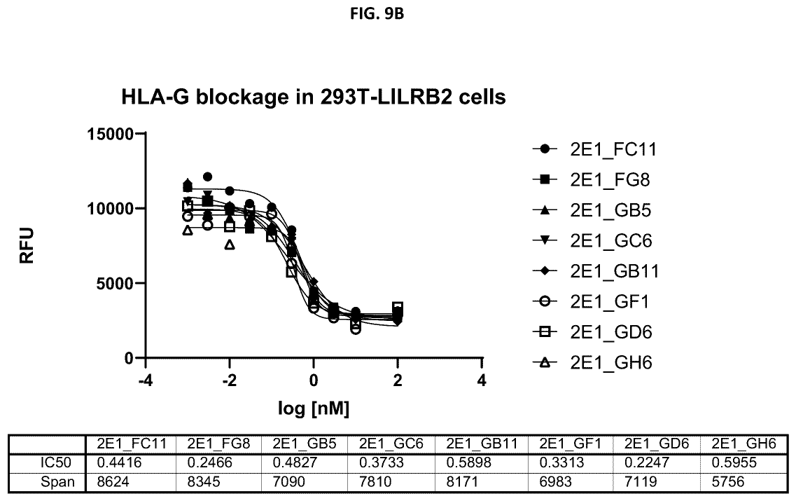

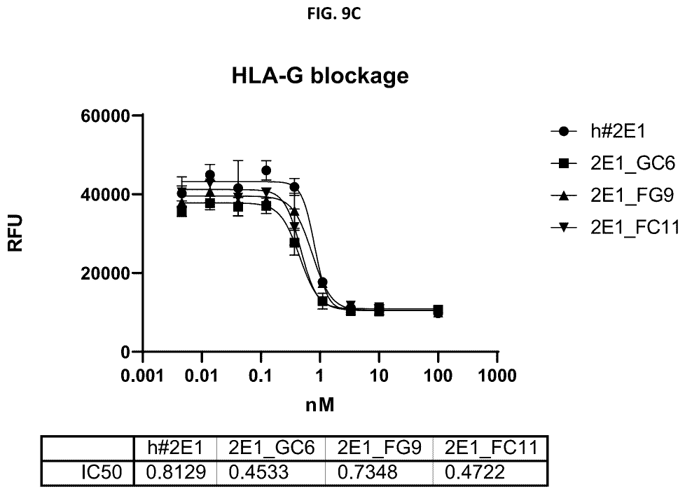

All tested matured anti-LILRB2 antibodies successfully blocked HLA-G binding to 293T-LILRB2 cells as shown in FIGS. 9A-9C.

TABLE 7
Binding Activity of Exemplary Matured Anti-LILRB2 Antibodies to Recombinant LILRB2 Protein

| clone # | 2E1 | 2E1_FC1 | 2E1_FG4 | 2E1_FG9 | 2E1_FE11 | 2E1_FD3 | 2E1_FG2 | 2E1_FC3 |
|---|---|---|---|---|---|---|---|---|
| Bmax | 1.386 | 1.348 | 1.397 | 1.37 | 1.233 | 1.279 | 1.261 | 1.286 |
| KD | 0.2673 | 0.2263 | 0.3206 | 0.2239 | 0.3004 | 0.2685 | 0.2866 | 0.2455 |

| clone # | 2E1 | 2E1_FH2 | 2E1_FF3 | 2E1_FD4 | 2E1_FF4 | 2E1_FF5 | 2E1_FD6 | 2E1_FF10 |
|---|---|---|---|---|---|---|---|---|
| Bmax | 1.411 | 0.9891 | 1.167 | 1.137 | 1.588 | 1.3 | 1.279 | 1.257 |
| KD | 0.1891 | 0.3578 | 0.3393 | 0.1386 | 0.1609 | 0.2149 | 0.2129 | 0.2071 |

| clone # | 2E1 | 2E1_FC11 | 2E1_FF9 | 2E1_FH9 | 2E1_FA1 | 2E1_FG8 | 2E1_FA11 | 2E1_FB11 |
|---|---|---|---|---|---|---|---|---|
| Bmax | 1.384 | 1.374 | 1.197 | 1.236 | 1.374 | 1.263 | 1.407 | 1.391 |
| KD | 0.2887 | 0.1665 | 0.2191 | 0.3168 | 0.2921 | 0.2753 | 0.3806 | 0.2039 |

| clone # | 2E1 | 2E1_GG3 | 2E1_GH3 | 2E1_GB5 | 2E1_GC6 | 2E1_GH9 | 2E1_GB11 | 2E1_GC11 |
|---|---|---|---|---|---|---|---|---|
| Bmax | 1.22 | 1.265 | 1.144 | 1.228 | 1.186 | 1.307 | 1.162 | 1.249 |
| KD | 0.2988 | 0.3376 | 0.2472 | 0.2556 | 0.1829 | 0.2925 | 0.3759 | 0.4239 |

| clone # | 2E1 | 2E1_GF1 | 2E1_GD2 | 2E1_GD6 | 2E1_GA9 | 2E1_GD9 | 2E1_GH11 | 2E1_GA1 |
|---|---|---|---|---|---|---|---|---|
| Bmax | 1.446 | 1.495 | 1.496 | 1.477 | 1.536 | 1.385 | 1.579 | 1.619 |
| KD | 0.2836 | 0.1562 | 0.3077 | 0.2066 | 0.2145 | 0.3373 | 0.2557 | 0.2544 |

| clone # | 2E1 | 2E1_GH4 | 2E1_GE6 | 2E1_GB7 | 2E1_GB8 | 2E1_GB1 | 2E1_GF9 | 2E1_GH6 |
|---|---|---|---|---|---|---|---|---|
| Bmax | 1.476 | 1.512 | 1.647 | 1.484 | 1.471 | 1.009 | 1.141 | 1.265 |
| KD | 0.3215 | 0.285 | 0.1971 | 0.2978 | 0.2093 | 0.3379 | 0.304 | 0.4379 |

| clone # | 2E1 | 2E1_GC8 | 2E1_GF8 | 2E1_GE11 | 2E1_GG1 | 2E1_GA4 |
|---|---|---|---|---|---|---|
| Bmax | 1.546 | 1.443 | 1.558 | 1.369 | 1.559 | 1.613 |
| KD | 0.2546 | 0.436 | 0.3075 | 0.2917 | 0.2749 | 0.2026 |

TABLE 8
Binding Activity of Exemplary Matured Anti-LILRB2 Antibodies to 293T-LILRB2 Cells

| clone # | 2E1 | 2E1_FC1 | 2E1_FG9 | 2E1_FD3 | 2E1_FC3 | 2E1_FF4 | 2E1_FF5 | 2E1_FC11 |
|---|---|---|---|---|---|---|---|---|
| Bmax | 488806 | 457474 | 463297 | 460283 | 455955 | 462390 | 468962 | 460659 |
| KD | 0.3166 | 0.2177 | 0.1937 | 0.1822 | 0.2199 | 0.272 | 0.2107 | 0.223 |
| clone # | 2E1 | 2E1_FG4 | 2E1_FE11 | 2E1_FG2 | 2E1_FH2 | 2E1_FF3 | 2E1_FD4 | 2E1_FD6 |
| Bmax | 424587 | 434159 | 450099 | 417182 | 420492 | 434449 | 431256 | 432055 |
| KD | 0.4205 | 0.4222 | 0.3824 | 0.2248 | 0.3489 | 0.4174 | 0.3285 | 0.3783 |
| clone # | 2E1 | 2E1_FF10 | 2E1_FF9 | 2E1_FH9 | 2E1_FA1 | 2E1_FG8 | 2E1_FA11 | 2E1_FB11 |
| Bmax | 646936 | 697956 | 708472 | 683836 | 688307 | 687849 | 697627 | 710089 |
| KD | 0.5857 | 0.256 | 0.2481 | 0.3126 | 0.2303 | 0.1364 | 0.1547 | 0.2343 |

| clone # | 2E1 | 2E1_GG3 | 2E1_GH3 | 2E1_GB5 | 2E1_GC6 | 2E1_GH9 | 2E1_GB11 | 2E1_GC11 |
|---|---|---|---|---|---|---|---|---|
| Bmax | 669643 | 641315 | 640563 | 645459 | 632059 | 636694 | 627241 | 640678 |
| KD | 0.2852 | 0.1336 | 0.09598 | 0.05471 | 0.06446 | 0.1336 | 0.06667 | 0.2326 |

| clone # | 2E1 | 2E1_GF1 | 2E1_GD2 | 2E1_GD6 | 2E1_GA9 | 2E1_GD9 | 2E1_GH11 | 2E1_GA1 |
|---|---|---|---|---|---|---|---|---|
| Bmax | 718377 | 706166 | 718492 | 698961 | 728650 | 723945 | 742850 | 739020 |
| KD | 0.32 | 0.1214 | 0.2206 | 0.1105 | 0.1387 | 0.1867 | 0.2354 | 0.2449 |
| clone # | 2E1 | 2E1_GH4 | 2E1_GE6 | 2E1_GB7 | 2E1_GB8 | 2E1_GB1 | 2E1_GF9 | 2E1_GE11 |
| Bmax | 645761 | 645923 | 664777 | 636227 | 659059 | 637456 | 638894 | 658029 |
| KD | 0.5605 | 0.4105 | 0.3142 | 0.3811 | 0.3318 | 0.379 | 0.2826 | 0.3581 |
| clone # | 2E1 | 2E1_GG1 | 2E1_GA4 | 2E1_GH6 | 2E1_FE1 | 2E1_GC8 | 2E1_GF8 | 2E1_GB5 |
| Bmax | 657930 | 687585 | 724806 | 678212 | 689825 | 696974 | 688058 | 670157 |
| KD | 0.5734 | 0.6657 | 0.6737 | 0.181 | 0.5656 | 0.519 | 0.4024 | 0.3767 |

(iii) Blockade of HLA-A2 Binding to 293T-LILRB2 Cells

0.2 ug His-tagged HLA-A2 tetramer was incubated with 293T-LILRB2 cells at room temperature for 45 minutes to allow for HLA-G/LILRB2 binding. Serial diluted h#2E1 antibodies were then added to the mixture and incubated at room temperature for another 45 minutes. The binding of HLA-G/LILRB2 was detected afterwards using a secondary PE-labeled anti-His tag antibody via flow cytometry.

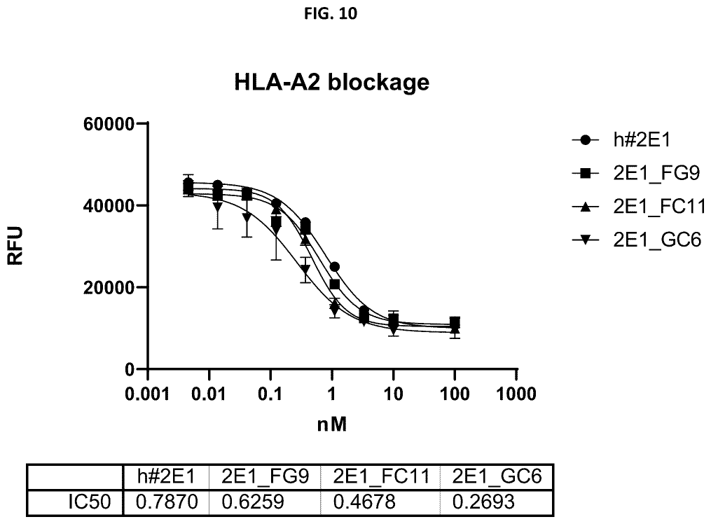

All tested matured anti-LILRB2 antibodies successfully blocked HLA-A2 binding to 293T-LILRB2 cells as shown in Table 9 below and FIG. 10.

TABLE 9
Blocking Activity of Exemplary Matured Anti-LILRB2 Antibodies
Against Binding of HLA-A2 to 293T-LILRB2 Cells

| | 2E1_FC1 | 2E1_FG4 | 2E1_FG9 | 2E1_FE11 | 2E1_FD3 | 2E1_FG2 | 2E1_FC3 |
|---|---|---|---|---|---|---|---|
| % no Ab | 56.70304506 | 55.37313441 | 53.50513504 | 50.12554443 | 50.9910761 | 53.5856898 | 52.1478084 |
| | 2E1_FF5 | 2E1_FD6 | 2E1_FF10 | 2E1_FC11 | 2E1_FF9 | 2E1_FH9 | 2E1_FA1 |
| % no Ab | 54.81016953 | 53.32249975 | 49.1531227 | 50.65424831 | 48.6257518 | 47.1341371 | 48.7549961 |
| | 2E1_GG3 | 2E1_GB5 | 2E1_GC6 | 2E1_GH9 | 2E1_GB11 | 2E1_GC11 | 2E1_GF1 |
| % no Ab | 53.34051289 | 50.6769809 | 48.30463143 | 53.40735968 | 50.1376493 | 45.128049 | 47.8529698 |
| | 2E1_GD9 | 2E1_GB1 | 2E1_GF9 | 2E1_GE11 | 2E1_GH6 | 2E1_FH2 | 2E1_FG8 |
| % no Ab | 52.90169462 | 50.94044119 | 46.20853157 | 46.1778732 | 46.6347767 | 51.46755991 | 47.24625096 |

| | 2E1_FD4 | 2E1_FF4 | 2E1_FA11 | 2E1_FB11 | 2E1_GD6 | 2E1_GA9 | 2E1_GD2 |
|---|---|---|---|---|---|---|---|
| % no Ab | 49.034272 | 53.25417588 | 49.3013349 | 47.69115768 | 45.5534475 | 45.3214021 | 51.93062388 |

### Example 4. Humanized Anti-LILRB2 Antibodies Induced Macrophage M1 Polarization in a PBMC/LPS Model

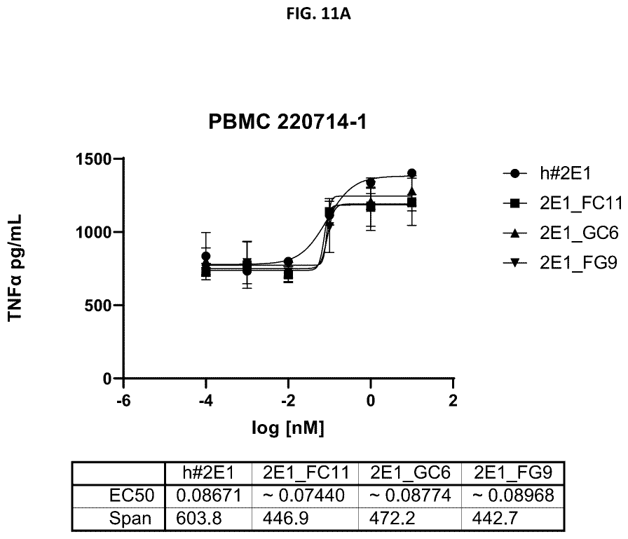

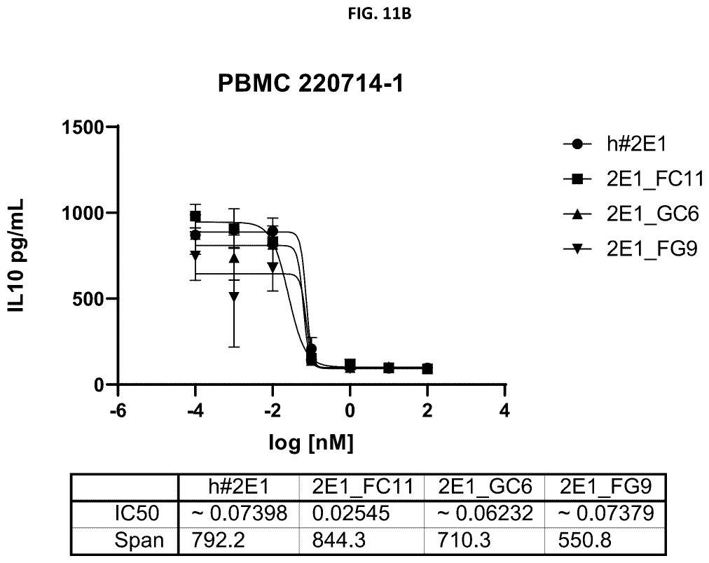

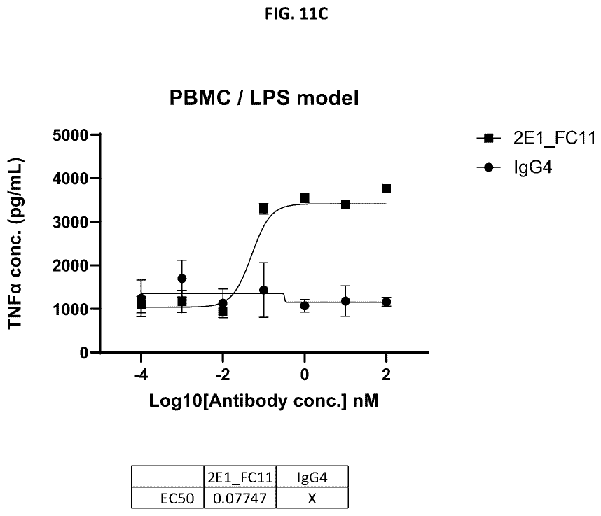

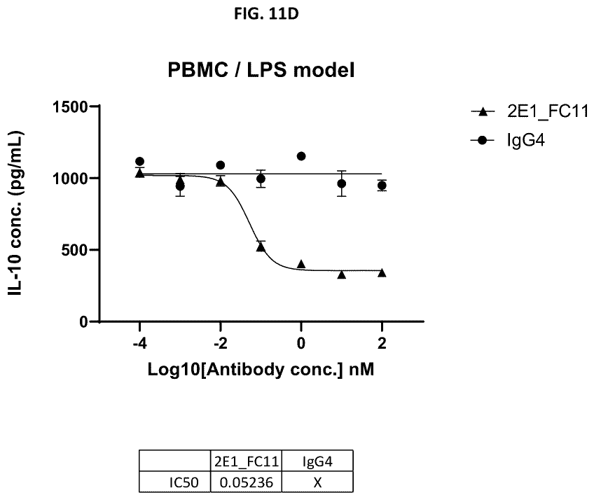

This example explores the ability of exemplary humanized anti-LILRB2 antibodies in inducing macrophage M1 polarization in a PBMC/LPS model. Briefly, freshly isolated human peripheral blood mononuclear cells (PBMCs) were cultured in RPMI1640 supplemented with 10% FBS. The cells were treated with exemplary humanized anti-LILRB2 antibodies, including clones 2E1, 2E1_FC11 (A16), 2E1_GC6 (A26), and 2E1_FG9 (A3), for 48 hours, and then stimulated with lipopolysaccharide (LPS) at 100 ng/ml for 24 hours. The culture supernatant was collected and examined for levels of hTNFα and IL10 using ELISA assays. As shown in FIGS. 11A-11D, the tested exemplary humanized anti-LILRB2 antibodies, specifically clone 2E1_FC11 (A16), induced macrophage polarization to a pro-inflammatory M1 stage as evidenced by the increase of TNFα levels and decrease of the IL10 levels.

### Example 5. Humanized Anti-LILRB2 Antibodies Induced Macrophage M1 Polarization in a Human Monocytes-Derived Macrophase HMDM/LPS Model

Human peripheral blood mononuclear cells (PBMCs) were isolated from a human donor following conventional practice. Monocytes were isolated from the PBMCs using anti-CD33 beads. The isolated monocytes were incubated in a medium containing M-CSF for 6 days to allow for differentiation into macrophages. The cells were then treated with clone 2E1_FC11 as an example, or with human IgG4 as a control, for 24 hours. The treated cells were stimulated by LPS at 100 ng/ml for 24 hours and the level of TNFα was measured in the culture supernatant by ELISA as a readout of M1 polarization.

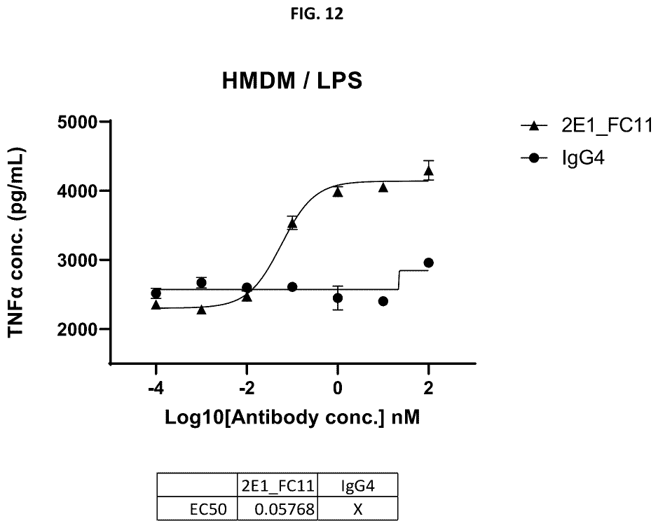

As shown in FIG. 12, clone 2E1_FC11 successfully induced macrophage M1 polarization as indicated by the secretion of TNFα in the culture supernatant.

### Example 6. Humanized Anti-LILRB2 Antibodies CD8+ T Cell Activation in an Autologous Mixed Lymphocyte Reaction Assay

Human peripheral blood mononuclear cells (PBMCs) were isolated from a human donor following conventional practice. CD33+ monocyte was isolated and differentiated to macrophage as 6 days culture with CSF and testing antibodies. CD8+ T cells were isolated and added into the differentiated macrophage with testing antibodies and an anti-CD3 antibody for 4 days culture. Single domain anti-PDL1 antibody A105 or anti-PD1 antibody nivolumab work as controls to activate T cells in auto-MLR systems. The levels of IFNγ and GM-CSF were measured in the culture supernatant using ELISA as a readout of T cell activation.

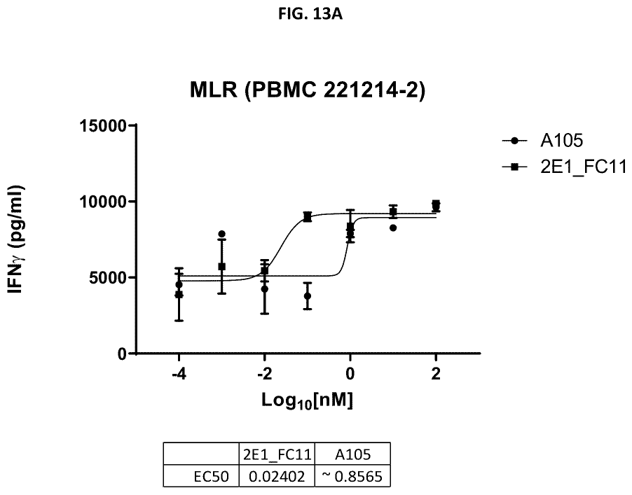

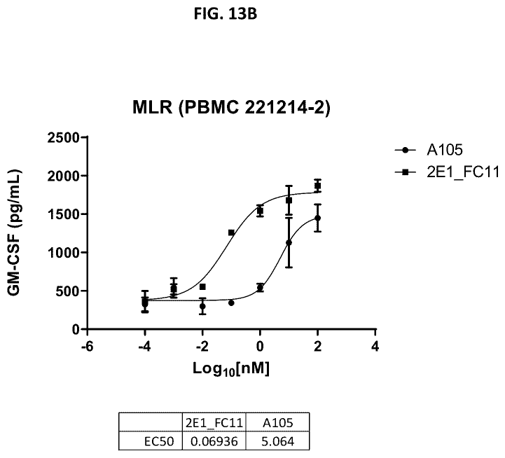

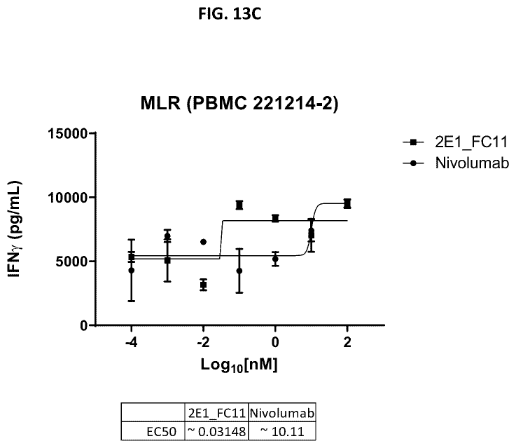

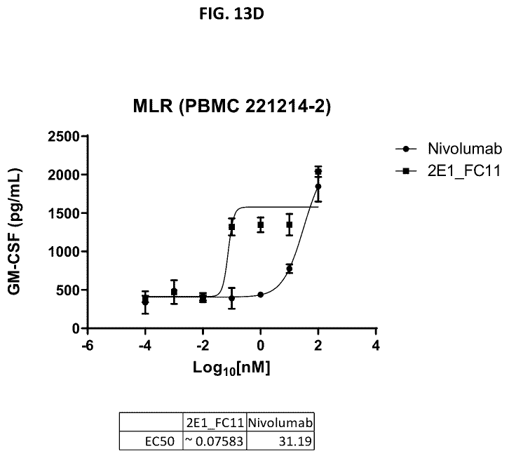

As shown in FIGS. 13A-13D, clone 2E1_FC11 showed better activity in inducing CD8+ T cell activation as compared with PD1 inhibitors A105 and nivolumab.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. An antibody that binds human leukocyte immunoglobulin-like receptor 2 (LILRB2) (anti-LILRB2 antibody), wherein the anti-LILRB2 antibody comprises:
(a) a heavy chain variable region (V_{H}), which comprises
(ai) a heavy chain CDR1 comprising GX₁SITSGYX₂WX₃ (SEQ ID NO 192), in which X₁ is Y or G, X₂ is Y, S, A, or H, and X₃ is N, S, or W;
(aii) a heavy chain CDR2 comprising X₄ISYDGNX₅X₆ (SEQ ID NO: 194), in which X₄ is S, T, or Y, X₅ is A, I, K, L, M, N, P, S, T, V, or W and X₆ is H, L, N, S, or W; optionally wherein the heavy chain CDR2 comprises X₄ISYDGNX₅X₆YNPSLKN (SEQ ID NO: 416);
(aiii) a heavy chain CDR3 comprising EEX₇TMX₈TTX₉WFX₁₀Y (SEQ ID NO: 196), in which X₇ is A or S, X₈ is F or I, X₉ is A or G, and X₁₀ is A, G, Q or S; and
(b) a light chain variable region (V_{L}), which comprises
(bi) a light chain CDR1 comprising X₁₁X₁₂SEX₁₃IX₁₄SNX₁₅A (SEQ ID NO: 199), in which X₁₁ is E, G, L, N, Q, R, S, T or V, X₁₂ is A or G, X₁₃ is K, N, T, or V, X₁₄ is F or Y, and X₁₅ is L, N, or Q;
(bii) a light chain CDR2 comprising GATX₁₆X₁₇X₁₈X₁₉ (SEQ ID NO: 201), in which X₁₆ is E, N, W, or Y, X₁₇ is F, H, L, P, or R, X₁₈ is A, N, R, or S, and X₁₉ is A, G, K, L, R, S, or V; and
(biii) a light chain CDR3 comprising QX₂₀FWDX₂₁PYX₂₂ (SEQ ID NO:
203), in which X₂₀ is H or Q, X₂₁ is L, S, T, or Y, and X₂₂ is I, M, R, T, Y, or V.

2. The antibody of claim 1, wherein X₁ is Y, X₂ is Y, X₃ is N, X₄ is Y, X₅ is I, X₆ is N, X₇ is S, X₈ is I, X₉ is A, X₁₀ is A, or a combination thereof; and/or wherein X₁₁ is R, X₁₂ is A, X₁₃ is N, X₁₄ is Y, X₁₅ is L, X₁₆ is N, X₁₇ is L, X₁ₛ is A, X₁₉ is S, X₂₀ is H, X₂₁ is Y, X₂₂ is T, or a combination thereof.

3. The antibody of claim 1 or claim 2, wherein the heavy chain CDR3 is EESTMITTAWFAY (SEQ ID NO: 11); and/or the light chain CDR3 is QHFWDYPYT (SEQ ID NO: 247).

4. The antibody of claim 1, wherein the anti-LILRB2 antibody comprises the same heavy chain CDR1, CDR2, and CDR3, and the same light chain CDR1, CDR2, and CDR3 as the antibodies listed in Table 2.

5. The antibody of claim 1, wherein the anti-LILRB2 antibody comprises the heavy chain CDR1, CDR2, and CDR3 set forth in SEQ ID NOs: 9, 243, and 11, respectively, and the light chain CDR1, CDR2, and CDR3 set forth in SEQ ID NOs: 13, 14, and 247.

6. The antibody of claim 1, wherein the heavy chain CDR1, CDR2, and CDR3 of the anti-LILRB2 antibody comprises up to five amino acid residue variations as relative to SEQ IDs: 9, 243, and 11, and/or the light chain CDR1, CDR2, and CDR3 of the anti-LILRB2 antibody comprises up to five amino acid variations relative to SEQ IDs: 13, 14, and 247.

7. The antibody of any one of claims 1-6, wherein the anti-LILRB2 antibody is a humanized antibody.

8. The antibody of claim 7, wherein the V_{H} comprises a V_{H} framework 1 (FR1), a V_{H} framework 2 (FR2), a V_{H} framework 3 (FR3), and a V_{H} framework 4 (FR4) and the V_{L} comprises a V_{L} FR1, a V_{L} FR2, a V_{L} FR3, and a V_{L} FR4;
wherein:
the V_{H} FR1 comprises EVQLVESGGGLVQPGGSLRLSCAZ₁S, in which Z₁ is A or V,
the V_{H} FR2 comprises WZ₂RQAPGKGLEWVA, in which Z₂ is I or V,
the V_{H} FR3 comprises RFTISRDZ₃SKNTLZ₄LQMNSLRAEDTAVYYCZ₅R, in which Z₃ is A, D or T, Z₄ is F, L, or V, and Z₅ is A or V, and
the V_{H} FR4 comprises WGQGTLVTVSS; and
wherein:
the V_{L} FR1 comprises DIQZ₆TQSPSSLSASVGDRVTITC, in which Z₆ is L or M,
the V_{L} FR2 comprises WYQQKPGKAPKLLIY,
the V_{L} FR3 comprises GVPSRFSGSGSGTDZ₇TLTISSLQPEDFATYYC, in which Z₇ is F or Y, and
the V_{L} FR4 comprises FGQGTKVEIK.

9. The antibody of claim 5, wherein the V_{H} of the anti-LILRB2 antibody comprises an amino acid sequence at least 85% identical to SEQ ID NO: 244; and/or wherein the V_{L} of the anti-LILRB2 antibody comprises an amino acid sequence at least 85% identical to SEQ ID NO: 248.

10. The antibody of claim 9, wherein the V_{H} of the anti-LILRB2 antibody comprises the amino acid sequence of SEQ ID NO: 244; and/or wherein the V_{L} of the anti-LILRB2 antibody comprises the amino acid sequence of SEQ ID NO: 248.

11. The antibody of claim 1, wherein the anti-LILRB2 antibody is one of those listed in Table 2.

12. The antibody of any one of claims 1-11, wherein anti-LILRB2 antibody is a full-length antibody or an antigen-binding fragment thereof.

13. A nucleic acid or a set of nucleic acids, comprising a nucleotide sequence(s) encoding the anti-LILRB2 antibody of any one of claims 1-12.

14. The nucleic acid or set of nucleic acids of claim 13, which is a vector or a set of vectors comprising the nucleotide sequence(s) encoding the anti-LiLRB2 antibody.

15. The nucleic acid or set of nucleic acids of claim 14, wherein the vector(s) is an expression vector(s).

16. A host cell or host cell set, comprising the nucleic acid(s) of any one of claims 13-15.

17. The host cell or host cell set of claim 16, which is a mammalian cell(s), a yeast cell(s), or a bacterial cell(s).

18. A pharmaceutical composition comprising (a) an anti-LILRB2 antibody of any one of claims 1-12, or a nucleic acid or set of nucleic acids encoding the antibody, and a pharmaceutically acceptable carrier.

19. A method for modulating immune responses, comprising administering to a subject in need thereof an effective amount of the anti-LILRB2 antibody of any one of claims 1-12, the nucleic acid or set of nucleic acids encoding the antibody, or a pharmaceutical composition comprising the antibody or the encoding nucleic acid(s).

20. The method of claim 19, wherein the subject is a human patient having or suspected of having cancer.

21. A method for preparing an anti-LILRB2 antibody, comprising:
culturing the host cell or host cell set of claim 16 or claim 17 under conditions allowing for expression of the antibody, and
harvesting the antibody thus produced.


SEQUENCE LISTING (416 sequences)

SEQ ID NO: 1 - protein, 10 aa, synthetic construct
GNSFTDNNIY

SEQ ID NO: 2 - protein, 17 aa, synthetic construct
YIDPFNGRTTYNQKFKG

SEQ ID NO: 3 - protein, 6 aa, synthetic construct
GDYFNY

SEQ ID NO: 4 - protein, 115 aa, synthetic construct
EIQLQQSGPELVKPGASVKVSCKASGNSFTDNNIYWVKQSHGESLEWIGYIDPFNGRTTYNQKFKGKATLTVDKSSSTAFMHLNSLTSGDSAVYYCARGDYFNYWGQGTTLTVSS

SEQ ID NO: 5 - protein, 11 aa, synthetic construct
SASQGINNYLN

SEQ ID NO: 6 - protein, 7 aa, synthetic construct
YTSSLHS

SEQ ID NO: 7 - protein, 9 aa, synthetic construct
QQYSNLPFT

SEQ ID NO: 8 - protein, 107 aa, synthetic construct
EIQMTQTTSSLSASLGDRVTISCSASQGINNYLNWFQQKSDGTVKLLIYYTSSLHSGVPSRFSGSGSGTDYSLTISNLEPEDIAIYYCQQYSNLPFTFGGGTELEIK

SEQ ID NO: 9 - protein, 11 aa, synthetic construct
GYSITSGYYWN

SEQ ID NO: 10 - protein, 16 aa, synthetic construct
YISYDGNNNYNPSLKN

SEQ ID NO: 11 - protein, 13 aa, synthetic construct
EESTMITTAWFAY

SEQ ID NO: 12 - protein, 122 aa, synthetic construct
DVQLQESGPGLVKPSQSLSLTCSVTGYSITSGYYWNWIRQFPGNKLEWMGYISYDGNNNYNPSLKNRISITRDTSKNQFFLKLNSVTTEDTATYYCAREESTMITTAWFAYWGQGTLVTVSA

SEQ ID NO: 13 - protein, 11 aa, synthetic construct
RASENIYSNLA

SEQ ID NO: 14 - protein, 7 aa, synthetic construct
GATNLAS

SEQ ID NO: 15 - protein, 9 aa, synthetic construct
QHFWDTPYT

SEQ ID NO: 16 - protein, 107 aa, synthetic construct
DIQMTQSPASLSVSVGETVTITCRASENIYSNLAWYQQKQGKSPQLLVYGATNLADGVPSRFSGSGSGTQYSLKINSLQSEDFGSYFCQHFWDTPYTFGGGTKLEIK

SEQ ID NO: 17 - protein, 10 aa, synthetic construct
GYAFTTYNMY

SEQ ID NO: 18 - protein, 17 aa, synthetic construct
YIDPYNGGATYNLKFKG

SEQ ID NO: 19 - protein, 115 aa, synthetic construct
EIQLQQSGPELVKPGASVKLSCKASGYAFTTYNMYWVKQSPGKSLEWIGYIDPYNGGATYNLKFKGKATLTVDKSSSAAYMHLNGLTSEDSAVYYCAGGDYFNYWGQGTILTVSS

SEQ ID NO: 20 - protein, 11 aa, synthetic construct
RASQVINNYLN

SEQ ID NO: 21 - protein, 7 aa, synthetic construct
YTSTLHS

SEQ ID NO: 22 - protein, 9 aa, synthetic construct
QQYSTLPYT

SEQ ID NO: 23 - protein, 107 aa, synthetic construct
DIQMTQTTSSLSASLGDRVTISCRASQVINNYLNWFQQKPDGTVKLLIYYTSTLHSGVPSRFSGSGSGTDYSLTISNLEPEDFATYYCQQYSTLPYTFGGGTKLEIK

SEQ ID NO: 24 - protein, 10 aa, synthetic construct
GSSFTDYNMY

SEQ ID NO: 25 - protein, 17 aa, synthetic construct
YIDPYNGRTTYNQKFKG

SEQ ID NO: 26 - protein, 6 aa, synthetic construct
GDYFDY

SEQ ID NO: 27 - protein, 115 aa, synthetic construct
EIQLQQSGPELVKPGASVKVSCKASGSSFTDYNMYWVKQIHGKSLDWIGYIDPYNGRTTYNQKFKGKATLTVDKSSSTAFMHLNSLTSEDSAVYYCAGGDYFDYWGHGTSLTVSS

SEQ ID NO: 28 - protein, 11 aa, synthetic construct
SASQGISNYLN

SEQ ID NO: 29 - protein, 9 aa, synthetic construct
QQYSNLPYT

SEQ ID NO: 30 - protein, 107 aa, synthetic construct
EIQMTQTTSSLSASLGDRVTISCSASQGISNYLNWFQQKPDGTVKLLIYYTSTLHSGVPSRFSGSGSGTDFSLTISNLDPEDFATYYCQQYSNLPYTFGGGTKLEIK

SEQ ID NO: 31 - protein, 10 aa, synthetic construct
GFTFSSYGMS

SEQ ID NO: 32 - protein, 17 aa, synthetic construct
TISGAFSYTYYPDSVKG

SEQ ID NO: 33 - protein, 11 aa, synthetic construct
HDNSLYYAMDY

SEQ ID NO: 34 - protein, 120 aa, synthetic construct
EVKLVESGGGLVKPGGSLKLSCAASGFTFSSYGMSWVRQTPEKRLEWVATISGAFSYTYYPDSVKGRFTISRDNAKNNLYLQMSSLRSEDTAFYYCARHDNSLYYAMDYWGQGTSVTVSS

SEQ ID NO: 35 - protein, 11 aa, synthetic construct
RTSKSITKYLA

SEQ ID NO: 36 - protein, 7 aa, synthetic construct
SGSTLQS

SEQ ID NO: 37 - protein, 9 aa, synthetic construct
QQHNEYPWT

SEQ ID NO: 38 - protein, 107 aa, synthetic construct
NVQITQSPSYLAASPGETITINCRTSKSITKYLAWYQEQPGKTNKLLIYSGSTLQSGIPSRFSGSGSGTDFTLTISSLEPEDFAMYYCQQHNEYPWTFGGGTKLEIK

SEQ ID NO: 39 - protein, 17 aa, synthetic construct
YIDPYNGRTTYNQKFRG

SEQ ID NO: 40 - protein, 115 aa, synthetic construct
EIQLQQSGPELVKPGTSVKVSCKASGSSFTDYNMYWVKQSHGKSLDWIGYIDPYNGRTTYNQKFRGKATLTVDKSSSTAFMHLNSLTSEDSAVYYCARGDYFDYWGQGTSLTVSS

SEQ ID NO: 41 - protein, 10 aa, synthetic construct
GFTFSTYGMS

SEQ ID NO: 42 - protein, 17 aa, synthetic construct
TISGSNSYTYYPDSVKG

SEQ ID NO: 43 - protein, 12 aa, synthetic construct
HENGYYFYAMDY

SEQ ID NO: 44 - protein, 121 aa, synthetic construct
EVKLVESGGDLVKPGGSLKLSCAASGFTFSTYGMSWVRQTPEKRLEWVATISGSNSYTYYPDSVKGRFTISRDNAKNNLYLQMSSLRSEDTALYYCTRHENGYYFYAMDYWGQGTSVTVSS

SEQ ID NO: 45 - protein, 11 aa, synthetic construct
RSSKSITKYLA

SEQ ID NO: 46 - protein, 9 aa, synthetic construct
QQHSEYPWT

SEQ ID NO: 47 - protein, 107 aa, synthetic construct
DVQITQSPSYLTASPGETITINCRSSKSITKYLAWYQEKPGKTNKLLIYSGSTLQSGIPSRFSGSGSGTDFTLTISSLEPEDFAMYYCQQHSEYPWTFGGGTRLEIK

SEQ ID NO: 48 - protein, 10 aa, synthetic construct
GYTFTNYGMN

SEQ ID NO: 49 - protein, 17 aa, synthetic construct
WMNTYIGEPTYADDFKG

SEQ ID NO: 50 - protein, 11 aa, synthetic construct
RGDYASYYFDY

SEQ ID NO: 51 - protein, 120 aa, synthetic construct
QIHLVQSGPELKKPGETVKISCKASGYTFTNYGMNWVKQVPGKALKWMGWMNTYIGEPTYADDFKGRFAFSLETSASTAYLQINNLKNEDMATYFCARRGDYASYYFDYWGQGTTLTVSS

SEQ ID NO: 52 - protein, 11 aa, synthetic construct
RASQSISNNLH

SEQ ID NO: 53 - protein, 7 aa, synthetic construct
YASQSIS

SEQ ID NO: 54 - protein, 9 aa, synthetic construct
QQSNSWPHT

SEQ ID NO: 55 - protein, 107 aa, synthetic construct
DIVLTQSPATLSVTPGDSVSLSCRASQSISNNLHWYHQKSHESPRLLIKYASQSISGIPSRVSGSGSGTDFTLSIITVETEDFGMYFCQQSNSWPHTFGGGTKLEIR

SEQ ID NO: 56 - protein, 17 aa, synthetic construct
WINTYTGEPTYADDFRG

SEQ ID NO: 57 - protein, 8 aa, synthetic construct
YYDYDYFD

SEQ ID NO: 58 - protein, 118 aa, synthetic construct
QIQLVQSGPELKKPGETVRISCKASGYTFTNYGMNWVKQAPGKGLKWMGWINTYTGEPTYADDFRGRFALSLEPSASTASLQINNLKNEDTATYFCARYYDYDYFDYWGQGTTLTVSS

SEQ ID NO: 59 - protein, 17 aa, synthetic construct
KSSQSLLNGGNQKNYLA

SEQ ID NO: 60 - protein, 7 aa, synthetic construct
GASTRVS

SEQ ID NO: 61 - protein, 9 aa, synthetic construct
QNDLSYPLT

SEQ ID NO: 62 - protein, 113 aa, synthetic construct
DIVMTQSPSSLRVSAGEKVTMSCKSSQSLLNGGNQKNYLAWYQQKPGQPPKVLIYGASTRVSGVPDRFTGRGSGIDFTLTISSVEAEDLAVYYCQNDLSYPLTFGAGTKLELK

SEQ ID NO: 63 - protein, 120 aa, synthetic construct
QIHLVQSGPELKKPGETVKISCKASGYTFTNYGMNWVKQAPGKALKWMGWMNTYIGEPTYADDFKGRFAFSLETSASTAYLQINNLKNEDMATYFCARRGDYASYYFDYWGQGTTLTVSS

SEQ ID NO: 64 - protein, 11 aa, synthetic construct
RASQNINNNLH

SEQ ID NO: 65 - protein, 107 aa, synthetic construct
DIVLTQSPATLSVTPGDSVSLSCRASQNINNNLHWYHQKSHESPRLLIKYASQSISGIPSRVSGSGSGTDFTLSIVTVETEDFGMFFCQQSNSWPHTFGGGTKLEIR

SEQ ID NO: 66 - protein, 10 aa, synthetic construct
GYAFTSHNIY

SEQ ID NO: 67 - protein, 17 aa, synthetic construct
YIDPYNGGSTYNQKFKD

SEQ ID NO: 68 - protein, 6 aa, synthetic construct
GDYRDF

SEQ ID NO: 69 - protein, 115 aa, synthetic construct
EIQLQQSGPELVKPGASVKVSCKASGYAFTSHNIYWVKQSHGKSLEWIGYIDPYNGGSTYNQKFKDKATLTVDKSSSTAYMHLKSLTSEDSAVYYCARGDYRDFWGQGTTLTVSS

SEQ ID NO: 70 - protein, 11 aa, synthetic construct
SASQAINNYLN

SEQ ID NO: 71 - protein, 9 aa, synthetic construct
QQYSHLPWT

SEQ ID NO: 72 - protein, 107 aa, synthetic construct
DIQMTQTTSSLSASLGDRVTIRCSASQAINNYLNWYQQKPDGTVKLLIYYTSSLHSGVPSRFSGSGSGADYSLTISNLEPEDIATYYCQQYSHLPWTFGGGTTLEIK

SEQ ID NO: 73 - protein, 10 aa, synthetic construct
GFTFSNFGMH

SEQ ID NO: 74 - protein, 17 aa, synthetic construct
YISSGSGTIYYSDTVRG

SEQ ID NO: 75 - protein, 11 aa, synthetic construct
SDYDGDWFFDV

SEQ ID NO: 76 - protein, 120 aa, synthetic construct
DVQLVESGGGLVQPGWSRILSCAASGFTFSNFGMHWVRQAPEKGLEWVAYISSGSGTIYYSDTVRGRFTISRDNPKNTLFLLMTSLRSEDTAMYYCTRSDYDGDWFFDVWGAGTTVTVSS

SEQ ID NO: 77 - protein, 11 aa, synthetic construct
KASQDVTTAVA

SEQ ID NO: 78 - protein, 7 aa, synthetic construct
SASFRYT

SEQ ID NO: 79 - protein, 9 aa, synthetic construct
QQHFTTPLT

SEQ ID NO: 80 - protein, 107 aa, synthetic construct
DIVMTQSHRFLSTSVGDRVTITCKASQDVTTAVAWCQQKPGQSPKLLIYSASFRYTGVPDRFTGSGSGTDFTFTINNVQAEDLALYYCQQHFTTPLTFGAGTKLELK

SEQ ID NO: 81 - protein, 10 aa, synthetic construct
GFTFSDFYMG

SEQ ID NO: 82 - protein, 19 aa, synthetic construct
ASGNKAHDYATEYSASVKG

SEQ ID NO: 83 - protein, 12 aa, synthetic construct
SYYYGSSAWFTY

SEQ ID NO: 84 - protein, 123 aa, synthetic construct
EVKLVEPGGGLVQPGGSLRLSCATSGFTFSDFYMGWVRQPPGKRLEWIAASGNKAHDYATEYSASVKGRFFVSRDTFQSILYLQMNALRAEDTAIYYCARSYYYGSSAWFTYWGQGTLVTVSV

SEQ ID NO: 85 - protein, 11 aa, synthetic construct
KASQDVGTAVA

SEQ ID NO: 86 - protein, 7 aa, synthetic construct
WASTRHA

SEQ ID NO: 87 - protein, 9 aa, synthetic construct
QQYSSYPLT

SEQ ID NO: 88 - protein, 107 aa, synthetic construct
DIVMTQSHKFMSTSIRDRVTIACKASQDVGTAVAWYQQKPGQSPKLLIFWASTRHAGVPDRFTGSGSGTDFTLTISNVQSEDLTDYFCQQYSSYPLTFGAGTKLELK

SEQ ID NO: 89 - protein, 10 aa, synthetic construct
GFSLTSYGVH

SEQ ID NO: 90 - protein, 16 aa, synthetic construct
VIWAGGTTNYNSALMS

SEQ ID NO: 91 - protein, 9 aa, synthetic construct
GGYGHSMDY

SEQ ID NO: 92 - protein, 117 aa, synthetic construct
QVQLKESGPGLVAPSQSLSITCTVSGFSLTSYGVHWVRQSPGKGLEWLGVIWAGGTTNYNSALMSRLSISKDNSKSQVFLKMNSLQADDTAMYYCARGGYGHSMDYWGQGTSVTVSS

SEQ ID NO: 93 - protein, 11 aa, synthetic construct
KASQDVSNAVA

SEQ ID NO: 94 - protein, 7 aa, synthetic construct
SASYRYT

SEQ ID NO: 95 - protein, 9 aa, synthetic construct
QQHYSTPFT

SEQ ID NO: 96 - protein, 107 aa, synthetic construct
DIVMTQSHKFMSTSVGDRVSITCKASQDVSNAVAWYQQKPGQSPKLLIYSASYRYTGVPDRFTGSGSGTDFIFTISSVQAEDLAVYYCQQHYSTPFTFGSGTKLEIK

SEQ ID NO: 97 - protein, 10 aa, synthetic construct
GYIFTDFNMH

SEQ ID NO: 98 - protein, 17 aa, synthetic construct
YIYPFNGGTGYNQKFEN

SEQ ID NO: 99 - protein, 9 aa, synthetic construct
RGGRGHFDY

SEQ ID NO: 100 - protein, 118 aa, synthetic construct
EVQLQQSGPDLVKPGASVKISCKASGYIFTDFNMHWVKQSHGKSLEWIGYIYPFNGGTGYNQKFENMATLTVDISSSTAYMEVRSLTSEDSAVYYCARRGGRGHFDYWGQGTTLTVSS

SEQ ID NO: 101 - protein, 11 aa, synthetic construct
RASGNIHNYLA

SEQ ID NO: 102 - protein, 7 aa, synthetic construct
NAKTLAD

SEQ ID NO: 103 - protein, 9 aa, synthetic construct
QHFWNTPFT

SEQ ID NO: 104 - protein, 107 aa, synthetic construct
DIQMTQSPASLSASVGETVTVTCRASGNIHNYLAWYQQKQGKSPQLLVYNAKTLADGVPSRFSGSGSGIQFSLKINSLQPEDFGSYYCQHFWNTPFTFGSGTKLEIK

SEQ ID NO: 105 - protein, 10 aa, synthetic construct
GFSLATYGVH

SEQ ID NO: 106 - protein, 16 aa, synthetic construct
VMWRDGSTDYNAVFMS

SEQ ID NO: 107 - protein, 15 aa, synthetic construct
NPYLYGSSHYYAMDY

SEQ ID NO: 108 - protein, 123 aa, synthetic construct
QVQLKQSGPGLVQPSQSLSITCTVSGFSLATYGVHWVRQSPGKGLEWLGVMWRDGSTDYNAVFMSRLSITKDDSKSQVFFKMNSLQADDTAIYYCAKNPYLYGSSHYYAMDYWGQGTSVTVSS

SEQ ID NO: 109 - protein, 17 aa, synthetic construct
KSSQSLLNSENQKNYLA

SEQ ID NO: 110 - protein, 7 aa, synthetic construct
GASTRES

SEQ ID NO: 111 - protein, 9 aa, synthetic construct
QNDHIYPFT

SEQ ID NO: 112 - protein, 114 aa, synthetic construct
GDIVMTQSPSSLSVSAGEKVTMSCKSSQSLLNSENQKNYLAWYQQKPGQPPKLLIYGASTRESGVPDRFTGSGSGTDFTLTISSVQAEDLAVYYCQNDHIYPFTFGSGTKLEIK

SEQ ID NO: 113 - protein, 10 aa, synthetic construct
GFSLTDYGVN

SEQ ID NO: 114 - protein, 16 aa, synthetic construct
MIWGDGNTDYNSALKS

SEQ ID NO: 115 - protein, 16 aa, synthetic construct
SSYRYDENIYYYAMDY

SEQ ID NO: 116 - protein, 124 aa, synthetic construct
QVQLKESGPGLVAPSQSLSITCTVSGFSLTDYGVNWVRQPPGKGLEWLGMIWGDGNTDYNSALKSRLSISKVNSKSQVFLKMNSLQTDDTARYYCARSSYRYDENIYYYAMDYWGQGTSVTVSS

SEQ ID NO: 117 - protein, 17 aa, synthetic construct
KSSQSLLNSGIQKNYLT

SEQ ID NO: 118 - protein, 7 aa, synthetic construct
WASTRES

SEQ ID NO: 119 - protein, 9 aa, synthetic construct
QNDYSLPFT

SEQ ID NO: 120 - protein, 113 aa, synthetic construct
DIVMTQSPSSLTVTAGEEVTLSCKSSQSLLNSGIQKNYLTWYQQKPGQPPKLLIYWASTRESGVPDRFTGSVSGTDFTLTISSVQAEDLAVYYCQNDYSLPFTFGSGTKLEIK

SEQ ID NO: 121 - protein, 10 aa, synthetic construct
GFNVKDYYMH

SEQ ID NO: 122 - protein, 17 aa, synthetic construct
WIDPKNGNGIYDPKFQG

SEQ ID NO: 123 - protein, 12 aa, synthetic construct
GDYFGSSYLFAY

SEQ ID NO: 124 - protein, 121 aa, synthetic construct
EVQLQQSGAELVRPGALVKLSCKASGFNVKDYYMHWVKQRPEQGLEWIGWIDPKNGNGIYDPKFQGKASITADTSSNTAYLQLSSLTSEDTSVYYCARGDYFGSSYLFAYWGQGTLVTVSA

SEQ ID NO: 125 - protein, 11 aa, synthetic construct
KASQDVSTAVA

SEQ ID NO: 126 - protein, 9 aa, synthetic construct
QQHYSTPWT

SEQ ID NO: 127 - protein, 107 aa, synthetic construct
DIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPWTFGGGAKLEIK

SEQ ID NO: 128 - protein, 16 aa, synthetic construct
VIWSGGITDYNAAFIS

SEQ ID NO: 129 - protein, 8 aa, synthetic construct
EIYGNPDY

SEQ ID NO: 130 - protein, 116 aa, synthetic construct
QVQLRQSGPGLVQPSQSLSITCTVSGFSLTSYGVHWVRQSPGKGLEWLGVIWSGGITDYNAAFISRLSISKDNSKSQVFFRMNSLQTNDTAIYYCAREIYGNPDYWGQGTTLTVSS

SEQ ID NO: 131 - protein, 11 aa, synthetic construct
KASQDVSIAVA

SEQ ID NO: 132 - protein, 9 aa, synthetic construct
QQHYSTPRT

SEQ ID NO: 133 - protein, 107 aa, synthetic construct
DIVMTQSHKFMSTSVGDRVSITCKASQDVSIAVAWFQQKPGQSPKLLIYSASYRYTGVPDRFSGSGSGTDFTFTISSVQAEDLAVYYCQQHYSTPRTFGGGTKLEIK

SEQ ID NO: 134 - protein, 10 aa, synthetic construct
GFSLTTFGVH

SEQ ID NO: 135 - protein, 16 aa, synthetic construct
VIWRGGSTDYNAAFMS

SEQ ID NO: 136 - protein, 15 aa, synthetic construct
NPYQYGSSHYYAMDY

SEQ ID NO: 137 - protein, 123 aa, synthetic construct
QVQLKQSGPGLVQPSQSLSITCTVSGFSLTTFGVHWVRQSPGKGLEWLGVIWRGGSTDYNAAFMSRLSITKDNSKSQVFFKMNSLQADDTAIYYCAKNPYQYGSSHYYAMDYWGQGTSVTVSS

SEQ ID NO: 138 - protein, 17 aa, synthetic construct
KSSQSLLNSGNQKNYLA

SEQ ID NO: 139 - protein, 9 aa, synthetic construct
QNDHILPFT

SEQ ID NO: 140 - protein, 113 aa, synthetic construct
DIVMTQSPSSLSVSAGEKVTMSCKSSQSLLNSGNQKNYLAWYQQKPGQPPKLLIYGASTRESGVPDRFTGSGSGTVFTLTISSVQAEDLAVYYCQNDHILPFTFGSGTKLEIK

SEQ ID NO: 141 - protein, 10 aa, synthetic construct
GFSLTTYGVH

SEQ ID NO: 142 - protein, 16 aa, synthetic construct
VIWTGGSTNYNSALMS

SEQ ID NO: 143 - protein, 9 aa, synthetic construct
GGYGHTMDY

SEQ ID NO: 144 - protein, 117 aa, synthetic construct
QVQLKESGPGLVAPSQSLSITCTVSGFSLTTYGVHWVRQPPGKGLEWLGVIWTGGSTNYNSALMSRLSIIKDNSKSQVFLKMNSLQTDDTAIYYCARGGYGHTMDYWGQGTSVTVSS

SEQ ID NO: 145 - protein, 107 aa, synthetic construct
DIVMTQSHKFMSTSVGDRVSITCKASQDVSIAVAWYQQKPGQFPKLLIYSASYRYTGVPDRFTGGGSGTDFTFTISSVQAEDLAVYYCQQHYSTPFTFGSGTKLEIK

SEQ ID NO: 146 - protein, 10 aa, synthetic construct
GFTFSDAWMD

SEQ ID NO: 147 - protein, 19 aa, synthetic construct
EIRSKSNNHTTYYAESVKG

SEQ ID NO: 148 - protein, 5 aa, synthetic construct
DWLTY

SEQ ID NO: 149 - protein, 116 aa, synthetic construct
EVKLEESGGGLVQPGGSMKLSCAASGFTFSDAWMDWVRQSPKKGLEWVAEIRSKSNNHTTYYAESVKGRFTISRDDSKSSVYLQMNSLRAEDTGIYYCTRDWLTYWGQGTLVTVSA

SEQ ID NO: 150 - protein, 15 aa, synthetic construct
RASKSVSTSGYSYMN

SEQ ID NO: 151 - protein, 7 aa, synthetic construct
LASNLES

SEQ ID NO: 152 - protein, 9 aa, synthetic construct
QHSRELPLT

SEQ ID NO: 153 - protein, 111 aa, synthetic construct
DIVLTQSPASLAVSLGQRATISCRASKSVSTSGYSYMNWYQQKPGQPPKLLIYLASNLESGVPARFSGSGSGTDFTLNIHPVEEEDAATYYCQHSRELPLTFGAGTKLELK

SEQ ID NO: 154 - protein, 10 aa, synthetic construct
GYTFTDYNMH

SEQ ID NO: 155 - protein, 17 aa, synthetic construct
SIYPYNGDTDYNQKFKS

SEQ ID NO: 156 - protein, 11 aa, synthetic construct
ENDYDGPPFPY

SEQ ID NO: 157 - protein, 120 aa, synthetic construct
EVQLQQSGPELVKPGASLKISCKASGYTFTDYNMHWVKQSHGKSLEWIGSIYPYNGDTDYNQKFKSKATLTIDNSSSTAFMDLRSLTSEDSAVYYCARENDYDGPPFPYWGLGTLVTVSA

SEQ ID NO: 158 - protein, 11 aa, synthetic construct
KASEDIYNRLA

SEQ ID NO: 159 - protein, 7 aa, synthetic construct
GATSLET

SEQ ID NO: 160 - protein, 8 aa, synthetic construct
QQYWSIRT

SEQ ID NO: 161 - protein, 106 aa, synthetic construct
DIQMTQSSSSFSVSLGDRVTITCKASEDIYNRLAWYQQKSGNAPRLLISGATSLETGVPSRFSGSGSGKDYTLSITSLQTEDVATYYCQQYWSIRTFGGGTKLEIK

SEQ ID NO: 162 - protein, 118 aa, synthetic construct
EVQLQQSGPDLVKPGASVKISCKASGYIFTDFNMHWVKQSHGKSLEWIGYIYPFNGGTGYNQKFENKATLTVDNSSSTAYMEVRSLTSEDSAVYYCARRGGRGHFDYWGQGTTLTVSS

SEQ ID NO: 163 - protein, 107 aa, synthetic construct
DIQMTQSPASLSASVGETVTVTCRASGNIHNYLAWYQQKQGKSPQLLVYNAKTLADGVPSRFSGSGSGTQFSLKINSLQPEDFGSYYCQHFWNTPFTFGSGTKLEIK

SEQ ID NO: 164 - protein, 10 aa, synthetic construct
GYTFTSYWMH

SEQ ID NO: 165 - protein, 17 aa, synthetic construct
EINPTNGRTNYNENFKS

SEQ ID NO: 166 - protein, 8 aa, synthetic construct
DTTATFAY

SEQ ID NO: 167 - protein, 117 aa, synthetic construct
QVQLQQPGAELVKPGASVKLSCKASGYTFTSYWMHWVKQRPGQGLEWIGEINPTNGRTNYNENFKSKATLTVDISSSTAYMQLSSLTSEDSAVYYCARDTTATFAYWGQGTLVTVSA

SEQ ID NO: 168 - protein, 11 aa, synthetic construct
RASDNINSNLA

SEQ ID NO: 169 - protein, 7 aa, synthetic construct
AAANLAD

SEQ ID NO: 170 - protein, 9 aa, synthetic construct
QHFWGTPYT

SEQ ID NO: 171 - protein, 107 aa, synthetic construct
DIQMTQSPASLSVSVGETVIITCRASDNINSNLAWYQQKQGKSPQLLVYAAANLADGVPSRFSGSGSGTQYSLKINSLQSEDFGTYYCQHFWGTPYTFGGGTKLEIK

SEQ ID NO: 172 - protein, 10 aa, synthetic construct
GYSFTDSNMY

SEQ ID NO: 173 - protein, 17 aa, synthetic construct
YIDPYNGGSRYNQKFKG

SEQ ID NO: 174 - protein, 115 aa, synthetic construct
EIQLQQSGPELVKPGASVKVSCKASGYSFTDSNMYWVKQSHGKSLEWIGYIDPYNGGSRYNQKFKGKATLTVDKSSSTAFMHLNSLTSGDSAVYYCARGDYFDYWGRGTTLTVSS

SEQ ID NO: 175 - protein, 7 aa, synthetic construct
YTSSLYS

SEQ ID NO: 176 - protein, 107 aa, synthetic construct
DIQMTQTTSSLSASLGDRVTISCSASQGISNYLNWYQQKPDGTVKLLIYYTSSLYSGVSSRFSGSGSGTDYSLTISNLEPEDIATYYCQQYSNLPYTFGGGTKLEIK

SEQ ID NO: 177 - protein, 10 aa, synthetic construct
GFTFSDFYME

SEQ ID NO: 178 - protein, 19 aa, synthetic construct
ASRNKANDYTTDYSASVKG

SEQ ID NO: 179 - protein, 10 aa, synthetic construct
VYYRYWYFDV

SEQ ID NO: 180 - protein, 121 aa, synthetic construct
EVKLVESGGGLVQPGGSLRLSCATSGFTFSDFYMEWVRQPPGERLEWIAASRNKANDYTTDYSASVKGRFIVSRDTSQSILYLQMNALRAEDTAIYYCARVYYRYWYFDVWGAGTTVTVSS

SEQ ID NO: 181 - protein, 11 aa, synthetic construct
KASQDVSFAVA

SEQ ID NO: 182 - protein, 7 aa, synthetic construct
WASTRHT

SEQ ID NO: 183 - protein, 9 aa, synthetic construct
QQHYSAPPT

SEQ ID NO: 184 - protein, 107 aa, synthetic construct
DIVMTQSHKFMSTFVGDRVSITCKASQDVSFAVAWYQRKPGQSPKLLIYWASTRHTGVPDRFTGSGSGTDYTLTISSVQAEDLALYYCQQHYSAPPTFGGGTKLEIK

SEQ ID NO: 185 - protein, 17 aa, synthetic construct
YIDPYNGGTRYNQKFKG

SEQ ID NO: 186 - protein, 6 aa, synthetic construct
GDYFDC

SEQ ID NO: 187 - protein, 115 aa, synthetic construct
EIQLQQSGPELVKPGASVKVSCKASGYSFTDSNMYWVKQSHGKSLEWIGYIDPYNGGTRYNQKFKGKATLTVDKSSSTAFMHLNSLTSEDSAVYYCARGDYFDCWGQGTTLTVSS

SEQ ID NO: 188 - protein, 107 aa, synthetic construct
DIQMTQTTSSLSASLGDRVTISCSASQGISNYLNWYQQKPDGTVKLLIYYTSSLYSGVSSRFSGSGSGTDYSLTISNLEPEDVATYYCQQYSNLPYTFGGGTKLEIK

SEQ ID NO: 189 - protein, 10 aa, synthetic construct
GYTFTSYRMH

SEQ ID NO: 190 - protein, 117 aa, synthetic construct
QVQLQQPGAELVKPGASVKLSCKASGYTFTSYRMHWVKQRPGQGLEWIGEINPTNGRTNYNENFKSKATLTVDKSSSTAYMQLSSLTSEDSAVYYCARDTTATFAYWGQGTLVTVSA

SEQ ID NO: 191 - protein, 25 aa, synthetic construct
  VARIANT 24: note = X is A or V
EVQLVESGGGLVQPGGSLRLSCAXS

SEQ ID NO: 192 - protein, 11 aa, synthetic construct
  VARIANT 2: note = X is Y or G
  VARIANT 9: note = X is Y, S, A, or H
  VARIANT 11: note = X is N, S, or W
GXSITSGYXWX

SEQ ID NO: 193 - protein, 14 aa, synthetic construct
  VARIANT 2: note = X is I or V
WXRQAPGKGLEWVA

SEQ ID NO: 194 - protein, 9 aa, synthetic construct
  VARIANT 1: note = X is S, T, or Y
  VARIANT 8: note = X is A, I, K, L, M, N, P, S, T, V, or W
  VARIANT 9: note = X is H, L, N, S, or W
XISYDGNXX

SEQ ID NO: 195 - protein, 32 aa, synthetic construct
  VARIANT 8: note = X is A, D, or T
  VARIANT 14: note = X is F, L, or V
  VARIANT 31: note = X is A or V
RFTISRDXSKNTLXLQMNSLRAEDTAVYYCXR

SEQ ID NO: 196 - protein, 13 aa, synthetic construct
  VARIANT 3: note = X is A or S
  VARIANT 6: note = X is F or I
  VARIANT 9: note = X is A or G
  VARIANT 12: note = X is A, G, Q, or S
EEXTMXTTXWFXY

SEQ ID NO: 197 - protein, 11 aa, synthetic construct
WGQGTLVTVSS

SEQ ID NO: 198 - protein, 23 aa, synthetic construct
  VARIANT 4: note = X is L or M
DIQXTQSPSSLSASVGDRVTITC

SEQ ID NO: 199 - protein, 11 aa, synthetic construct
  VARIANT 1: note = X is E, G, L, N, Q, R, S, T, or V
  VARIANT 2: note = X is A or G
  VARIANT 5: note = X is K, N, T, or V
  VARIANT 7: note = X is F or Y
  VARIANT 10: note = X is L, N, or Q
XXSEXIXSNXA

SEQ ID NO: 200 - protein, 15 aa, synthetic construct
WYQQKPGKAPKLLIY

SEQ ID NO: 201 - skipped

SEQ ID NO: 202 - protein, 32 aa, synthetic construct
  VARIANT 15: note = X is F or Y
GVPSRFSGSGSGTDXTLTISSLQPEDFATYYC

SEQ ID NO: 203 - protein, 9 aa, synthetic construct
  VARIANT 2: note = X is H or Q
  VARIANT 6: note = X is L, S, T, or Y
  VARIANT 9: note = X is I, M, R, T, Y, or V
QXFWDXPYX

SEQ ID NO: 204 - protein, 10 aa, synthetic construct
FGQGTKVEIK

SEQ ID NO: 205 - protein, 122 aa, synthetic construct
EVQLVESGGGLVQPGGSLRLSCAASGYSITSGYYWNWVRQAPGKGLEWVAYISYDGNNNYNPSLKNRFTISRDDSKNTLYLQMNSLRAEDTAVYYCAREESTMITTAWFAYWGQGTLVTVSS

SEQ ID NO: 206 - protein, 107 aa, synthetic construct
DIQMTQSPSSLSASVGDRVTITCRASENIYSNLAWYQQKPGKAPKLLIYGATNLASGVPSRFSGSGSGTDFTLTISSLQPEDFATYYCQHFWDTPYTFGQGTKVEIK

SEQ ID NO: 207 - protein, 122 aa, synthetic construct
EVQLVESGGGLVQPGGSLRLSCAVSGYSITSGYYWNWIRQAPGKGLEWVAYISYDGNNNYNPSLKNRFTISRDASKNTVYLQMNSLRAEDTAVYYCAREESTMITTAWFAYWGQGTLVTVSS

SEQ ID NO: 208 - protein, 107 aa, synthetic construct
DIQMTQSPSSLSASVGDRVTITCRASENIYSNLAWYQQKPGKAPKLLIYGATNLASGVPSRFSGSGSGTDYTLTISSLQPEDFATYYCQHFWDTPYTFGQGTKVEIK

SEQ ID NO: 209 - protein, 122 aa, synthetic construct
EVQLVESGGGLVQPGGSLRLSCAVSGYSITSGYYWNWVRQAPGKGLEWVAYISYDGNNNYNPSLKNRFTISRDTSKNTFYLQMNSLRAEDTAVYYCAREESTMITTAWFAYWGQGTLVTVSS

SEQ ID NO: 210 - protein, 122 aa, synthetic construct
EVQLVESGGGLVQPGGSLRLSCAVSGYSITSGYYWNWVRQAPGKGLEWVAYISYDGNNNYNPSLKNRFTISRDTSKNTVYLQMNSLRAEDTAVYYCVREESTMITTAWFAYWGQGTLVTVSS

SEQ ID NO: 211 - protein, 122 aa, synthetic construct
EVQLVESGGGLVQPGGSLRLSCAVSGYSITSGYYWNWIRQAPGKGLEWVAYISYDGNNNYNPSLKNRFTISRDASKNTFYLQMNSLRAEDTAVYYCAREESTMITTAWFAYWGQGTLVTVSS

SEQ ID NO: 212 - protein, 122 aa, synthetic construct
EVQLVESGGGLVQPGGSLRLSCAVSGYSITSGYYWNWVRQAPGKGLEWVAYISYDGNNNYNPSLKNRFTISRDTSKNTFYLQMNSLRAEDTAVYYCVREESTMITTAWFAYWGQGTLVTVSS

SEQ ID NO: 213 - protein, 122 aa, synthetic construct
EVQLVESGGGLVQPGGSLRLSCAVSGYSITSGYYWNWIRQAPGKGLEWVAYISYDGNNNYNPSLKNRFTISRDTSKNTFYLQMNSLRAEDTAVYYCAREESTMITTAWFAYWGQGTLVTVSS

SEQ ID NO: 214 - protein, 122 aa, synthetic construct
EVQLVESGGGLVQPGGSLRLSCAVSGYSITSGYYWNWIRQAPGKGLEWVAYISYDGNNNYNPSLKNRFTISRDTSKNTVYLQMNSLRAEDTAVYYCAREESTMITTAWFAYWGQGTLVTVSS

SEQ ID NO: 215 - protein, 107 aa, synthetic construct
DIQLTQSPSSLSASVGDRVTITCRASENIYSNLAWYQQKPGKAPKLLIYGATNLASGVPSRFSGSGSGTDYTLTISSLQPEDFATYYCQHFWDTPYTFGQGTKVEIK

SEQ ID NO: 216 - protein, 122 aa, synthetic construct
EVQLVESGGGLVQPGGSLRLSCAVSGYSITSGYYWNWVRQAPGKGLEWVAYISYDGNNNYNPSLKNRFTISRDASKNTFYLQMNSLRAEDTAVYYCAREESTMITTAWFAYWGQGTLVTVSS

SEQ ID NO: 217 - protein, 16 aa, synthetic construct
YISYDGNVNYNPSLKN

SEQ ID NO: 218 - protein, 122 aa, synthetic construct
EVQLVESGGGLVQPGGSLRLSCAVSGYSITSGYYWNWIRQAPGKGLEWVAYISYDGNVNYNPSLKNRFTISRDTSKNTFYLQMNSLRAEDTAVYYCAREESTMITTAWFAYWGQGTLVTVSS

SEQ ID NO: 219 - protein, 9 aa, synthetic construct
QHFWDTPYI

SEQ ID NO: 220 - protein, 107 aa, synthetic construct
DIQMTQSPSSLSASVGDRVTITCRASENIYSNLAWYQQKPGKAPKLLIYGATNLASGVPSRFSGSGSGTDYTLTISSLQPEDFATYYCQHFWDTPYIFGQGTKVEIK

SEQ ID NO: 221 - protein, 9 aa, synthetic construct
QHFWDTPYR

SEQ ID NO: 222 - protein, 107 aa, synthetic construct
DIQMTQSPSSLSASVGDRVTITCRASENIYSNLAWYQQKPGKAPKLLIYGATNLASGVPSRFSGSGSGTDYTLTISSLQPEDFATYYCQHFWDTPYRFGQGTKVEIK

SEQ ID NO: 223 - protein, 9 aa, synthetic construct
QHFWDTPYY

SEQ ID NO: 224 - protein, 107 aa, synthetic construct
DIQMTQSPSSLSASVGDRVTITCRASENIYSNLAWYQQKPGKAPKLLIYGATNLASGVPSRFSGSGSGTDYTLTISSLQPEDFATYYCQHFWDTPYYFGQGTKVEIK

SEQ ID NO: 225 - protein, 16 aa, synthetic construct
YISYDGNMNYNPSLKN

SEQ ID NO: 226 - protein, 122 aa, synthetic construct
EVQLVESGGGLVQPGGSLRLSCAVSGYSITSGYYWNWIRQAPGKGLEWVAYISYDGNMNYNPSLKNRFTISRDTSKNTFYLQMNSLRAEDTAVYYCAREESTMITTAWFAYWGQGTLVTVSS

SEQ ID NO: 227 - protein, 16 aa, synthetic construct
YISYDGNANYNPSLKN

SEQ ID NO: 228 - protein, 122 aa, synthetic construct
EVQLVESGGGLVQPGGSLRLSCAVSGYSITSGYYWNWIRQAPGKGLEWVAYISYDGNANYNPSLKNRFTISRDTSKNTFYLQMNSLRAEDTAVYYCAREESTMITTAWFAYWGQGTLVTVSS

SEQ ID NO: 229 - protein, 16 aa, synthetic construct
YISYDGNTNYNPSLKN

SEQ ID NO: 230 - protein, 122 aa, synthetic construct
EVQLVESGGGLVQPGGSLRLSCAVSGYSITSGYYWNWIRQAPGKGLEWVAYISYDGNTNYNPSLKNRFTISRDTSKNTFYLQMNSLRAEDTAVYYCAREESTMITTAWFAYWGQGTLVTVSS

SEQ ID NO: 231 - protein, 9 aa, synthetic construct
QHFWDTPYV

SEQ ID NO: 232 - protein, 107 aa, synthetic construct
DIQMTQSPSSLSASVGDRVTITCRASENIYSNLAWYQQKPGKAPKLLIYGATNLASGVPSRFSGSGSGTDYTLTISSLQPEDFATYYCQHFWDTPYVFGQGTKVEIK

SEQ ID NO: 233 - protein, 16 aa, synthetic construct
SISYDGNNNYNPSLKN

SEQ ID NO: 234 - protein, 122 aa, synthetic construct
EVQLVESGGGLVQPGGSLRLSCAVSGYSITSGYYWNWIRQAPGKGLEWVASISYDGNNNYNPSLKNRFTISRDTSKNTFYLQMNSLRAEDTAVYYCAREESTMITTAWFAYWGQGTLVTVSS

SEQ ID NO: 235 - protein, 16 aa, synthetic construct
YISYDGNNSYNPSLKN

SEQ ID NO: 236 - protein, 122 aa, synthetic construct
EVQLVESGGGLVQPGGSLRLSCAVSGYSITSGYYWNWIRQAPGKGLEWVAYISYDGNNSYNPSLKNRFTISRDTSKNTFYLQMNSLRAEDTAVYYCAREESTMITTAWFAYWGQGTLVTVSS

SEQ ID NO: 237 - protein, 16 aa, synthetic construct
YISYDGNPNYNPSLKN

SEQ ID NO: 238 - protein, 122 aa, synthetic construct
EVQLVESGGGLVQPGGSLRLSCAVSGYSITSGYYWNWIRQAPGKGLEWVAYISYDGNPNYNPSLKNRFTISRDTSKNTFYLQMNSLRAEDTAVYYCAREESTMITTAWFAYWGQGTLVTVSS

SEQ ID NO: 239 - protein, 16 aa, synthetic construct
YISYDGNNWYNPSLKN

SEQ ID NO: 240 - protein, 122 aa, synthetic construct
EVQLVESGGGLVQPGGSLRLSCAVSGYSITSGYYWNWIRQAPGKGLEWVAYISYDGNNWYNPSLKNRFTISRDTSKNTFYLQMNSLRAEDTAVYYCAREESTMITTAWFAYWGQGTLVTVSS

SEQ ID NO: 241 - protein, 16 aa, synthetic construct
YISYDGNKNYNPSLKN

SEQ ID NO: 242 - protein, 122 aa, synthetic construct
EVQLVESGGGLVQPGGSLRLSCAVSGYSITSGYYWNWIRQAPGKGLEWVAYISYDGNKNYNPSLKNRFTISRDTSKNTFYLQMNSLRAEDTAVYYCAREESTMITTAWFAYWGQGTLVTVSS

SEQ ID NO: 243 - protein, 16 aa, synthetic construct
YISYDGNINYNPSLKN

SEQ ID NO: 244 - protein, 122 aa, synthetic construct
EVQLVESGGGLVQPGGSLRLSCAVSGYSITSGYYWNWIRQAPGKGLEWVAYISYDGNINYNPSLKNRFTISRDTSKNTFYLQMNSLRAEDTAVYYCAREESTMITTAWFAYWGQGTLVTVSS

SEQ ID NO: 245 - protein, 9 aa, synthetic construct
QHFWDTPYM

SEQ ID NO: 246 - protein, 107 aa, synthetic construct
DIQMTQSPSSLSASVGDRVTITCRASENIYSNLAWYQQKPGKAPKLLIYGATNLASGVPSRFSGSGSGTDYTLTISSLQPEDFATYYCQHFWDTPYMFGQGTKVEIK

SEQ ID NO: 247 - protein, 9 aa, synthetic construct
QHFWDYPYT

SEQ ID NO: 248 - protein, 107 aa, synthetic construct
DIQMTQSPSSLSASVGDRVTITCRASENIYSNLAWYQQKPGKAPKLLIYGATNLASGVPSRFSGSGSGTDYTLTISSLQPEDFATYYCQHFWDYPYTFGQGTKVEIK

SEQ ID NO: 249 - protein, 16 aa, synthetic construct
YISYDGNLNYNPSLKN

SEQ ID NO: 250 - protein, 122 aa, synthetic construct
EVQLVESGGGLVQPGGSLRLSCAVSGYSITSGYYWNWIRQAPGKGLEWVAYISYDGNLNYNPSLKNRFTISRDTSKNTFYLQMNSLRAEDTAVYYCAREESTMITTAWFAYWGQGTLVTVSS

SEQ ID NO: 251 - protein, 16 aa, synthetic construct
YISYDGNNLYNPSLKN

SEQ ID NO: 252 - protein, 122 aa, synthetic construct
EVQLVESGGGLVQPGGSLRLSCAVSGYSITSGYYWNWIRQAPGKGLEWVAYISYDGNNLYNPSLKNRFTISRDTSKNTFYLQMNSLRAEDTAVYYCAREESTMITTAWFAYWGQGTLVTVSS

SEQ ID NO: 253 - protein, 16 aa, synthetic construct
YISYDGNSNYNPSLKN

SEQ ID NO: 254 - protein, 13 aa, synthetic construct
EESTMITTAWFQY

SEQ ID NO: 255 - protein, 122 aa, synthetic construct
EVQLVESGGGLVQPGGSLRLSCAVSGYSITSGYYWNWIRQAPGKGLEWVAYISYDGNSNYNPSLKNRFTISRDTSKNTFYLQMNSLRAEDTAVYYCAREESTMITTAWFQYWGQGTLVTVSS

SEQ ID NO: 256 - protein, 13 aa, synthetic construct
EEATMITTAWFAY

SEQ ID NO: 257 - protein, 122 aa, synthetic construct
EVQLVESGGGLVQPGGSLRLSCAVSGYSITSGYYWNWIRQAPGKGLEWVAYISYDGNVNYNPSLKNRFTISRDTSKNTFYLQMNSLRAEDTAVYYCAREEATMITTAWFAYWGQGTLVTVSS

SEQ ID NO: 258 - protein, 13 aa, synthetic construct
EESTMITTAWFSY

SEQ ID NO: 259 - protein, 122 aa, synthetic construct
EVQLVESGGGLVQPGGSLRLSCAVSGYSITSGYYWNWIRQAPGKGLEWVAYISYDGNVNYNPSLKNRFTISRDTSKNTFYLQMNSLRAEDTAVYYCAREESTMITTAWFSYWGQGTLVTVSS

SEQ ID NO: 260 - protein, 16 aa, synthetic construct
YISYDGNNHYNPSLKN

SEQ ID NO: 261 - protein, 13 aa, synthetic construct
EESTMFTTAWFQY

SEQ ID NO: 262 - protein, 122 aa, synthetic construct
EVQLVESGGGLVQPGGSLRLSCAVSGYSITSGYYWNWIRQAPGKGLEWVAYISYDGNNHYNPSLKNRFTISRDTSKNTFYLQMNSLRAEDTAVYYCAREESTMFTTAWFQYWGQGTLVTVSS

SEQ ID NO: 263 - protein, 7 aa, synthetic construct
GATNLAG

SEQ ID NO: 264 - protein, 107 aa, synthetic construct
DIQMTQSPSSLSASVGDRVTITCRASENIYSNLAWYQQKPGKAPKLLIYGATNLAGGVPSRFSGSGSGTDYTLTISSLQPEDFATYYCQHFWDTPYTFGQGTKVEIK

SEQ ID NO: 265 - protein, 7 aa, synthetic construct
GATNLSS

SEQ ID NO: 266 - protein, 107 aa, synthetic construct
DIQMTQSPSSLSASVGDRVTITCRASENIYSNLAWYQQKPGKAPKLLIYGATNLSSGVPSRFSGSGSGTDYTLTISSLQPEDFATYYCQHFWDTPYTFGQGTKVEIK

SEQ ID NO: 267 - protein, 7 aa, synthetic construct
GATNLNS

SEQ ID NO: 268 - protein, 107 aa, synthetic construct
DIQMTQSPSSLSASVGDRVTITCRASENIYSNLAWYQQKPGKAPKLLIYGATNLNSGVPSRFSGSGSGTDYTLTISSLQPEDFATYYCQHFWDTPYTFGQGTKVEIK

SEQ ID NO: 269 - protein, 7 aa, synthetic construct
GATNHAS

SEQ ID NO: 270 - protein, 107 aa, synthetic construct
DIQMTQSPSSLSASVGDRVTITCRASENIYSNLAWYQQKPGKAPKLLIYGATNHASGVPSRFSGSGSGTDYTLTISSLQPEDFATYYCQHFWDTPYTFGQGTKVEIK

SEQ ID NO: 271 - protein, 7 aa, synthetic construct
GATWLAS

SEQ ID NO: 272 - protein, 107 aa, synthetic construct
DIQMTQSPSSLSASVGDRVTITCRASENIYSNLAWYQQKPGKAPKLLIYGATWLASGVPSRFSGSGSGTDYTLTISSLQPEDFATYYCQHFWDTPYTFGQGTKVEIK

SEQ ID NO: 273 - protein, 7 aa, synthetic construct
GATNLAL

SEQ ID NO: 274 - protein, 107 aa, synthetic construct
DIQMTQSPSSLSASVGDRVTITCRASENIYSNLAWYQQKPGKAPKLLIYGATNLALGVPSRFSGSGSGTDYTLTISSLQPEDFATYYCQHFWDTPYTFGQGTKVEIK

SEQ ID NO: 275 - protein, 7 aa, synthetic construct
GATNLAR

SEQ ID NO: 276 - protein, 107 aa, synthetic construct
DIQMTQSPSSLSASVGDRVTITCRASENIYSNLAWYQQKPGKAPKLLIYGATNLARGVPSRFSGSGSGTDYTLTISSLQPEDFATYYCQHFWDTPYTFGQGTKVEIK

SEQ ID NO: 277 - protein, 11 aa, synthetic construct
RASEVIYSNLA

SEQ ID NO: 278 - protein, 107 aa, synthetic construct
DIQMTQSPSSLSASVGDRVTITCRASEVIYSNLAWYQQKPGKAPKLLIYGATNLASGVPSRFSGSGSGTDYTLTISSLQPEDFATYYCQHFWDTPYTFGQGTKVEIK

SEQ ID NO: 279 - protein, 12 aa, synthetic construct
TASENIYSNLAW

SEQ ID NO: 280 - protein, 7 aa, synthetic construct
GATNPAS

SEQ ID NO: 281 - protein, 107 aa, synthetic construct
DIQMTQSPSSLSASVGDRVTITCTASENIYSNLAWYQQKPGKAPKLLIYGATNPASGVPSRFSGSGSGTDYTLTISSLQPEDFATYYCQHFWDTPYTFGQGTKVEIK

SEQ ID NO: 282 - protein, 11 aa, synthetic construct
RASEKIYSNLA

SEQ ID NO: 283 - protein, 7 aa, synthetic construct
GATNLPS

SEQ ID NO: 284 - protein, 107 aa, synthetic construct
DIQMTQSPSSLSASVGDRVTITCRASEKIYSNLAWYQQKPGKAPKLLIYGATNLPSGVPSRFSGSGSGTDYTLTISSLQPEDFATYYCQHFWDTPYTFGQGTKVEIK

SEQ ID NO: 285 - protein, 11 aa, synthetic construct
EASENIYSNLA

SEQ ID NO: 286 - protein, 107 aa, synthetic construct
DIQMTQSPSSLSASVGDRVTITCEASENIYSNLAWYQQKPGKAPKLLIYGATNLASGVPSRFSGSGSGTDYTLTISSLQPEDFATYYCQHFWDTPYTFGQGTKVEIK

SEQ ID NO: 287 - protein, 11 aa, synthetic construct
RASENIFSNLA

SEQ ID NO: 288 - protein, 107 aa, synthetic construct
DIQMTQSPSSLSASVGDRVTITCRASENIFSNLAWYQQKPGKAPKLLIYGATNLASGVPSRFSGSGSGTDYTLTISSLQPEDFATYYCQHFWDTPYTFGQGTKVEIK

SEQ ID NO: 289 - protein, 11 aa, synthetic construct
GYSITSGYYWW

SEQ ID NO: 290 - protein, 9 aa, synthetic construct
YISYDGNNN

SEQ ID NO: 291 - protein, 122 aa, synthetic construct
EVQLVESGGGLVQPGGSLRLSCAVSGYSITSGYYWWWIRQAPGKGLEWVAYISYDGNNNYNPSLKNRFTISRDTSKNTFYLQMNSLRAEDTAVYYCAREESTMITTAWFAYWGQGTLVTVSS

SEQ ID NO: 292 - protein, 11 aa, synthetic construct
NASENIYSNLA

SEQ ID NO: 293 - protein, 107 aa, synthetic construct
DIQMTQSPSSLSASVGDRVTITCNASENIYSNLAWYQQKPGKAPKLLIYGATNLASGVPSRFSGSGSGTDYTLTISSLQPEDFATYYCQHFWDTPYTFGQGTKVEIK

SEQ ID NO: 294 - protein, 7 aa, synthetic construct
GATNFAS

SEQ ID NO: 295 - protein, 107 aa, synthetic construct
DIQMTQSPSSLSASVGDRVTITCRASENIYSNLAWYQQKPGKAPKLLIYGATNFASGVPSRFSGSGSGTDYTLTISSLQPEDFATYYCQHFWDTPYTFGQGTKVEIK

SEQ ID NO: 296 - protein, 11 aa, synthetic construct
RASENIYSNNA

SEQ ID NO: 297 - protein, 7 aa, synthetic construct
GATYLAS

SEQ ID NO: 298 - protein, 107 aa, synthetic construct
DIQMTQSPSSLSASVGDRVTITCRASENIYSNNAWYQQKPGKAPKLLIYGATYLASGVPSRFSGSGSGTDYTLTISSLQPEDFATYYCQHFWDTPYTFGQGTKVEIK

SEQ ID NO: 299 - protein, 11 aa, synthetic construct
LASENIYSNLA

SEQ ID NO: 300 - protein, 107 aa, synthetic construct
DIQMTQSPSSLSASVGDRVTITCLASENIYSNLAWYQQKPGKAPKLLIYGATNLASGVPSRFSGSGSGTDYTLTISSLQPEDFATYYCQHFWDTPYTFGQGTKVEIK

SEQ ID NO: 301 - protein, 11 aa, synthetic construct
SASENIYSNLA

SEQ ID NO: 302 - protein, 107 aa, synthetic construct
DIQMTQSPSSLSASVGDRVTITCSASENIYSNLAWYQQKPGKAPKLLIYGATNLASGVPSRFSGSGSGTDYTLTISSLQPEDFATYYCQHFWDTPYTFGQGTKVEIK

SEQ ID NO: 303 - protein, 11 aa, synthetic construct
RASENIYSNQA

SEQ ID NO: 304 - protein, 7 aa, synthetic construct
GATNRAS

SEQ ID NO: 305 - protein, 107 aa, synthetic construct
DIQMTQSPSSLSASVGDRVTITCRASENIYSNQAWYQQKPGKAPKLLIYGATNRASGVPSRFSGSGSGTDYTLTISSLQPEDFATYYCQHFWDTPYTFGQGTKVEIK

SEQ ID NO: 306 - protein, 11 aa, synthetic construct
RGSENIYSNLA

SEQ ID NO: 307 - protein, 107 aa, synthetic construct
DIQMTQSPSSLSASVGDRVTITCRGSENIYSNLAWYQQKPGKAPKLLIYGATNLASGVPSRFSGSGSGTDYTLTISSLQPEDFATYYCQHFWDTPYTFGQGTKVEIK

SEQ ID NO: 308 - protein, 11 aa, synthetic construct
GYSITSGYSWN

SEQ ID NO: 309 - protein, 122 aa, synthetic construct
EVQLVESGGGLVQPGGSLRLSCAVSGYSITSGYSWNWIRQAPGKGLEWVAYISYDGNNNYNPSLKNRFTISRDTSKNTFYLQMNSLRAEDTAVYYCAREESTMITTAWFAYWGQGTLVTVSS

SEQ ID NO: 310 - protein, 11 aa, synthetic construct
RASETIYSNLA

SEQ ID NO: 311 - protein, 7 aa, synthetic construct
GATNLAA

SEQ ID NO: 312 - protein, 107 aa, synthetic construct
DIQMTQSPSSLSASVGDRVTITCRASETIYSNLAWYQQKPGKAPKLLIYGATNLAAGVPSRFSGSGSGTDYTLTISSLQPEDFATYYCQHFWDTPYTFGQGTKVEIK

SEQ ID NO: 313 - protein, 11 aa, synthetic construct
GASENIYSNLA

SEQ ID NO: 314 - protein, 7 aa, synthetic construct
GATELAS

SEQ ID NO: 315 - protein, 107 aa, synthetic construct
DIQMTQSPSSLSASVGDRVTITCGASENIYSNLAWYQQKPGKAPKLLIYGATELASGVPSRFSGSGSGTDYTLTISSLQPEDFATYYCQHFWDTPYTFGQGTKVEIK

SEQ ID NO: 316 - protein, 11 aa, synthetic construct
GYSITSGYAWN

SEQ ID NO: 317 - protein, 122 aa, synthetic construct
EVQLVESGGGLVQPGGSLRLSCAVSGYSITSGYAWNWIRQAPGKGLEWVAYISYDGNNNYNPSLKNRFTISRDTSKNTFYLQMNSLRAEDTAVYYCAREESTMITTAWFAYWGQGTLVTVSS

SEQ ID NO: 318 - protein, 11 aa, synthetic construct
VASENIYSNLA

SEQ ID NO: 319 - protein, 107 aa, synthetic construct
DIQMTQSPSSLSASVGDRVTITCVASENIYSNLAWYQQKPGKAPKLLIYGATNLASGVPSRFSGSGSGTDYTLTISSLQPEDFATYYCQHFWDTPYTFGQGTKVEIK

SEQ ID NO: 320 - protein, 11 aa, synthetic construct
GGSITSGYYWW

SEQ ID NO: 321 - protein, 122 aa, synthetic construct
EVQLVESGGGLVQPGGSLRLSCAVSGGSITSGYYWWWIRQAPGKGLEWVAYISYDGNNNYNPSLKNRFTISRDTSKNTFYLQMNSLRAEDTAVYYCAREESTMITTAWFAYWGQGTLVTVSS

SEQ ID NO: 322 - protein, 7 aa, synthetic construct
GATNLAK

SEQ ID NO: 323 - protein, 107 aa, synthetic construct
DIQMTQSPSSLSASVGDRVTITCRASENIYSNLAWYQQKPGKAPKLLIYGATNLAKGVPSRFSGSGSGTDYTLTISSLQPEDFATYYCQHFWDTPYTFGQGTKVEIK

SEQ ID NO: 324 - protein, 11 aa, synthetic construct
GYSITSGYHWN

SEQ ID NO: 325 - protein, 122 aa, synthetic construct
EVQLVESGGGLVQPGGSLRLSCAVSGYSITSGYHWNWIRQAPGKGLEWVAYISYDGNNNYNPSLKNRFTISRDTSKNTFYLQMNSLRAEDTAVYYCAREESTMITTAWFAYWGQGTLVTVSS

SEQ ID NO: 326 - protein, 9 aa, synthetic construct
YISYDGNTN

SEQ ID NO: 327 - protein, 9 aa, synthetic construct
QQFWDTPYT

SEQ ID NO: 328 - protein, 107 aa, synthetic construct
DIQMTQSPSSLSASVGDRVTITCRASENIYSNLAWYQQKPGKAPKLLIYGATNLASGVPSRFSGSGSGTDYTLTISSLQPEDFATYYCQQFWDTPYTFGQGTKVEIK

SEQ ID NO: 329 - protein, 11 aa, synthetic construct
QASENIYSNLA

SEQ ID NO: 330 - protein, 107 aa, synthetic construct
DIQMTQSPSSLSASVGDRVTITCQASENIYSNLAWYQQKPGKAPKLLIYGATYLASGVPSRFSGSGSGTDYTLTISSLQPEDFATYYCQHFWDTPYTFGQGTKVEIK

SEQ ID NO: 331 - protein, 11 aa, synthetic construct
GYSITSGYYWS

SEQ ID NO: 332 - protein, 122 aa, synthetic construct
EVQLVESGGGLVQPGGSLRLSCAVSGYSITSGYYWSWIRQAPGKGLEWVAYISYDGNNNYNPSLKNRFTISRDTSKNTFYLQMNSLRAEDTAVYYCAREESTMITTAWFAYWGQGTLVTVSS

SEQ ID NO: 333 - protein, 9 aa, synthetic construct
YISYDGNVN

SEQ ID NO: 334 - protein, 9 aa, synthetic construct
QHFWDSPYT

SEQ ID NO: 335 - protein, 107 aa, synthetic construct
DIQMTQSPSSLSASVGDRVTITCRASENIYSNLAWYQQKPGKAPKLLIYGATNLASGVPSRFSGSGSGTDYTLTISSLQPEDFATYYCQHFWDSPYTFGQGTKVEIK

SEQ ID NO: 336 - protein, 9 aa, synthetic construct
TISYDGNPN

SEQ ID NO: 337 - protein, 122 aa, synthetic construct
EVQLVESGGGLVQPGGSLRLSCAVSGYSITSGYYWNWIRQAPGKGLEWVATISYDGNPNYNPSLKNRFTISRDTSKNTFYLQMNSLRAEDTAVYYCAREESTMITTAWFAYWGQGTLVTVSS

SEQ ID NO: 338 - protein, 7 aa, synthetic construct
GATNLAV

SEQ ID NO: 339 - protein, 107 aa, synthetic construct
DIQMTQSPSSLSASVGDRVTITCRASENIYSNLAWYQQKPGKAPKLLIYGATNLAVGVPSRFSGSGSGTDYTLTISSLQPEDFATYYCQHFWDTPYTFGQGTKVEIK

SEQ ID NO: 340 - protein, 9 aa, synthetic construct
YISYDGNIN

SEQ ID NO: 341 - protein, 9 aa, synthetic construct
YISYDGNSN

SEQ ID NO: 342 - protein, 122 aa, synthetic construct
EVQLVESGGGLVQPGGSLRLSCAVSGYSITSGYYWNWIRQAPGKGLEWVAYISYDGNSNYNPSLKNRFTISRDTSKNTFYLQMNSLRAEDTAVYYCAREESTMITTAWFAYWGQGTLVTVSS

SEQ ID NO: 343 - protein, 9 aa, synthetic construct
YISYDGNNW

SEQ ID NO: 344 - protein, 13 aa, synthetic construct
EESTMITTGWFAY

SEQ ID NO: 345 - protein, 122 aa, synthetic construct
EVQLVESGGGLVQPGGSLRLSCAVSGYSITSGYYWNWIRQAPGKGLEWVAYISYDGNNWYNPSLKNRFTISRDTSKNTFYLQMNSLRAEDTAVYYCAREESTMITTGWFAYWGQGTLVTVSS

SEQ ID NO: 346 - protein, 9 aa, synthetic construct
QHFWDLPYT

SEQ ID NO: 347 - protein, 107 aa, synthetic construct
DIQMTQSPSSLSASVGDRVTITCRASENIYSNLAWYQQKPGKAPKLLIYGATNLASGVPSRFSGSGSGTDYTLTISSLQPEDFATYYCQHFWDLPYTFGQGTKVEIK

SEQ ID NO: 348 - protein, 9 aa, synthetic construct
YISYDGNWN

SEQ ID NO: 349 - protein, 13 aa, synthetic construct
EESTMFTTAWFAY

SEQ ID NO: 350 - protein, 122 aa, synthetic construct
EVQLVESGGGLVQPGGSLRLSCAVSGYSITSGYYWNWIRQAPGKGLEWVAYISYDGNWNYNPSLKNRFTISRDTSKNTFYLQMNSLRAEDTAVYYCAREESTMFTTAWFAYWGQGTLVTVSS

SEQ ID NO: 351 - protein, 9 aa, synthetic construct
SISYDGNNN

SEQ ID NO: 352 - protein, 13 aa, synthetic construct
EESTMITTAWFGY

SEQ ID NO: 353 - protein, 122 aa, synthetic construct
EVQLVESGGGLVQPGGSLRLSCAVSGYSITSGYYWNWIRQAPGKGLEWVASISYDGNNNYNPSLKNRFTISRDTSKNTFYLQMNSLRAEDTAVYYCAREESTMITTAWFGYWGQGTLVTVSS

SEQ ID NO: 354 - protein, 25 aa, synthetic construct
  VARIANT 10: note = X is D or G
  VARIANT 24: note = X is A, T, or V
EVQLVESGGXLVQPGGSLRLSCAXS

SEQ ID NO: 355 - protein, 32 aa, synthetic construct
  VARIANT 2: note = X is A, F, I, L, S, or V
  VARIANT 8: note = X is D or N
  VARIANT 31: note = X is A or S
RXTISRDXSKNTLYLQMNSLRAEDTAVYYCXR

SEQ ID NO: 356 - protein, 15 aa, synthetic construct
  VARIANT 2: note = X is F or Y
WXQQKPGKAPKLLIY

SEQ ID NO: 357 - protein, 32 aa, synthetic construct
  VARIANT 15: note = X is F or Y
  VARIANT 27: note = X is F or L
GVPSRFSGSGSGTDXTLTISSLQPEDXATYYC

SEQ ID NO: 358 - protein, 120 aa, synthetic construct
EVQLVESGGGLVQPGGSLRLSCAASGFTFSNFGMHWVRQAPGKGLEWVAYISSGSGTIYYSDTVRGRFTISRDDSKNTLYLQMNSLRAEDTAVYYCARSDYDGDWFFDVWGQGTLVTVSS

SEQ ID NO: 359 - protein, 107 aa, synthetic construct
DIQMTQSPSSLSASVGDRVTITCKASQDVTTAVAWYQQKPGKAPKLLIYSASFRYTGVPSRFSGSGSGTDFTLTISSLQPEDFATYYCQQHFTTPLTFGQGTKVEIK

SEQ ID NO: 360 - protein, 120 aa, synthetic construct
EVQLVESGGGLVQPGGSLRLSCAASGFTFSNFGMHWVRQAPGKGLEWVAYISSGSGTIYYSDTVRGRVTISRDNSKNTLYLQMNSLRAEDTAVYYCARSDYDGDWFFDVWGQGTLVTVSS

SEQ ID NO: 361 - protein, 107 aa, synthetic construct
DIQLTQSPSSLSASVGDRVTITCKASQDVTTAVAWYQQKPGKAPKLLIYSASFRYTGVPSRFSGSGSGTDFTLTISSLQPEDFATYYCQQHFTTPLTFGQGTKVEIK

SEQ ID NO: 362 - protein, 120 aa, synthetic construct
EVQLVESGGGLVQPGGSLRLSCATSGFTFSNFGMHWVRQAPGKGLEWVAYISSGSGTIYYSDTVRGRFTISRDNSKNTLYLQMNSLRAEDTAVYYCARSDYDGDWFFDVWGQGTLVTVSS

SEQ ID NO: 363 - protein, 107 aa, synthetic construct
DIQMTQSPSSLSASVGDRVTITCKASQDVTTAVAWFQQKPGKAPKLLIYSASFRYTGVPSRFSGSGSGTDFTLTISSLQPEDFATYYCQQHFTTPLTFGQGTKVEIK

SEQ ID NO: 364 - protein, 120 aa, synthetic construct
EVQLVESGGGLVQPGGSLRLSCAASGFTFSNFGMHWVRQAPGKGLEWVAYISSGSGTIYYSDTVRGRFTISRDNSKNTLYLQMNSLRAEDTAVYYCARSDYDGDWFFDVWGQGTLVTVSS

SEQ ID NO: 365 - protein, 107 aa, synthetic construct
DIQMTQSPSSLSASVGDRVTITCKASQDVTTAVAWYQQKPGKAPKLLIYSASFRYTGVPSRFSGSGSGTDYTLTISSLQPEDFATYYCQQHFTTPLTFGQGTKVEIK

SEQ ID NO: 366 - protein, 107 aa, synthetic construct
DIQMTQSPSSLSASVGDRVTITCKASQDVTTAVAWYQQKPGKAPKLLIYSASFRYTGVPSRFSGSGSGTDFTLTISSLQPEDLATYYCQQHFTTPLTFGQGTKVEIK

SEQ ID NO: 367 - protein, 120 aa, synthetic construct
EVQLVESGGGLVQPGGSLRLSCATSGFTFSNFGMHWIRQAPGKGLEWVAYISSGSGTIYYSDTVRGRFTISRDNSKNTLYLQMNSLRAEDTAVYYCARSDYDGDWFFDVWGQGTLVTVSS

SEQ ID NO: 368 - protein, 107 aa, synthetic construct
DIQMTQSPSSLSASVGDRVTITCKASQDVTTAVAWFQQKPGKAPKLLIYSASFRYTGVPSRFSGSGSGTDFTLTISSLQPEDLATYYCQQHFTTPLTFGQGTKVEIK

SEQ ID NO: 369 - protein, 120 aa, synthetic construct
EVQLVESGGDLVQPGGSLRLSCAASGFTFSNFGMHWVRQAPGKGLEWVAYISSGSGTIYYSDTVRGRFTISRDNSKNTLYLQMNSLRAEDTAVYYCARSDYDGDWFFDVWGQGTLVTVSS

SEQ ID NO: 370 - protein, 120 aa, synthetic construct
EVQLVESGGGLVQPGGSLRLSCAASGFTFSNFGMHWVRQAPGKGLEWVAYISSGSGTIYYSDTVRGRITISRDNSKNTLYLQMNSLRAEDTAVYYCARSDYDGDWFFDVWGQGTLVTVSS

SEQ ID NO: 371 - protein, 120 aa, synthetic construct
EVQLVESGGGLVQPGGSLRLSCATSGFTFSNFGMHWVRQAPGKGLEWVAYISSGSGTIYYSDTVRGRLTISRDNSKNTLYLQMNSLRAEDTAVYYCARSDYDGDWFFDVWGQGTLVTVSS

SEQ ID NO: 372 - protein, 120 aa, synthetic construct
EVQLVESGGGLVQPGGSLRLSCATSGFTFSNFGMHWVRQAPGKGLEWVAYISSGSGTIYYSDTVRGRATISRDNSKNTLYLQMNSLRAEDTAVYYCSRSDYDGDWFFDVWGQGTLVTVSS

SEQ ID NO: 373 - protein, 107 aa, synthetic construct
DIQLTQSPSSLSASVGDRVTITCKASQDVTTAVAWFQQKPGKAPKLLIYSASFRYTGVPSRFSGSGSGTDYTLTISSLQPEDLATYYCQQHFTTPLTFGQGTKVEIK

SEQ ID NO: 374 - protein, 120 aa, synthetic construct
EVQLVESGGGLVQPGGSLRLSCAASGFTFSNFGMHWVRQAPGKGLEWVAYISSGSGTIYYSDTVRGRLTISRDNSKNTLYLQMNSLRAEDTAVYYCARSDYDGDWFFDVWGQGTLVTVSS

SEQ ID NO: 375 - protein, 120 aa, synthetic construct
EVQLVESGGGLVQPGGSLRLSCAASGFTFSNFGMHWVRQAPGKGLEWVAYISSGSGTIYYSDTVRGRSTISRDNSKNTLYLQMNSLRAEDTAVYYCARSDYDGDWFFDVWGQGTLVTVSS

SEQ ID NO: 376 - protein, 120 aa, synthetic construct
EVQLVESGGGLVQPGGSLRLSCAASGFTFSNFGMHWIRQAPGKGLEWVAYISSGSGTIYYSDTVRGRFTISRDNSKNTLYLQMNSLRAEDTAVYYCARSDYDGDWFFDVWGQGTLVTVSS

SEQ ID NO: 377 - protein, 120 aa, synthetic construct
EVQLVESGGGLVQPGGSLRLSCATSGFTFSNFGMHWVRQAPGKGLEWVAYISSGSGTIYYSDTVRGRITISRDNSKNTLYLQMNSLRAEDTAVYYCSRSDYDGDWFFDVWGQGTLVTVSS

SEQ ID NO: 378 - protein, 120 aa, synthetic construct
EVQLVESGGGLVQPGGSLRLSCAVSGFTFSNFGMHWVRQAPGKGLEWVAYISSGSGTIYYSDTVRGRFTISRDNSKNTLYLQMNSLRAEDTAVYYCARSDYDGDWFFDVWGQGTLVTVSS

SEQ ID NO: 379 - protein, 25 aa, synthetic construct
  VARIANT 24: note = X is A, I, or V
EVQLVESGGGLVQPGGSLRLSCAXS

SEQ ID NO: 380 - protein, 15 aa, synthetic construct
VITGGSTNYNSALMS

SEQ ID NO: 381 - protein, 32 aa, synthetic construct
  VARIANT 2: note = X is A, F, I, T, or V
  VARIANT 6: note = X is K or R
  VARIANT 8: note = X is D or N
  VARIANT 11: note = X is A, L or V
  VARIANT 13: note = X is A, L, or V
  VARIANT 32: note = X is K or R
RXTISXDXSKXTXYLQMNSLRAEDTAVYYCAX

SEQ ID NO: 382 - protein, 7 aa, synthetic construct
SASYLYS

SEQ ID NO: 383 - protein, 32 aa, synthetic construct
  VARIANT 27: note = X is F or L
GVPSRFSGSGSGTDFTLTISSLQPEDXATYYC

SEQ ID NO: 384 - protein, 117 aa, synthetic construct
EVQLVESGGGLVQPGGSLRLSCAASGFSLTTYGVHWVRQAPGKGLEWVAVIWTGGSTNYNSALMSRFTISRDDSKNTLYLQMNSLRAEDTAVYYCARGGYGHTMDYWGQGTLVTVSS

SEQ ID NO: 385 - protein, 107 aa, synthetic construct
DIQMTQSPSSLSASVGDRVTITCKASQDVSIAVAWYQQKPGKAPKLLIYSASYLYSGVPSRFSGSGSGTDFTLTISSLQPEDFATYYCQQHYSTPFTFGQGTKVEIK

SEQ ID NO: 386 - protein, 117 aa, synthetic construct
EVQLVESGGGLVQPGGSLRLSCAASGFSLTTYGVHWVRQAPGKGLEWVAVIWTGGSTNYNSALMSRFTISKDDSKNTLYLQMNSLRAEDTAVYYCARGGYGHTMDYWGQGTLVTVSS

SEQ ID NO: 387 - protein, 107 aa, synthetic construct
DIQMTQSPSSLSASVGDRVTITCKASQDVSIAVAWYQQKPGKAPKLLIYSASYLYSGVPSRFSGSGSGTDFTLTISSLQPEDLATYYCQQHYSTPFTFGQGTKVEIK

SEQ ID NO: 388 - protein, 117 aa, synthetic construct
EVQLVESGGGLVQPGGSLRLSCAASGFSLTTYGVHWVRQAPGKGLEWVAVIWTGGSTNYNSALMSRFTISKDDSKNTVYLQMNSLRAEDTAVYYCAKGGYGHTMDYWGQGTLVTVSS

SEQ ID NO: 389 - protein, 117 aa, synthetic construct
EVQLVESGGGLVQPGGSLRLSCAVSGFSLTTYGVHWVRQAPGKGLEWVAVIWTGGSTNYNSALMSRFTISKDDSKNTLYLQMNSLRAEDTAVYYCAKGGYGHTMDYWGQGTLVTVSS

SEQ ID NO: 390 - protein, 107 aa, synthetic construct
DIQLTQSPSSLSASVGDRVTITCKASQDVSIAVAWYQQKPGKAPKLLIYSASYLYSGVPSRFSGSGSGTDFTLTISSLQPEDFATYYCQQHYSTPFTFGQGTKVEIK

SEQ ID NO: 391 - protein, 117 aa, synthetic construct
EVQLVESGGGLVQPGGSLRLSCAVSGFSLTTYGVHWVRQAPGKGLEWVAVIWTGGSTNYNSALMSRTTISKDNSKNTVYLQMNSLRAEDTAVYYCAKGGYGHTMDYWGQGTLVTVSS

SEQ ID NO: 392 - protein, 117 aa, synthetic construct
EVQLVESGGGLVQPGGSLRLSCAVSGFSLTTYGVHWVRQAPGKGLEWVAVIWTGGSTNYNSALMSRATISKDNSKNTVYLQMNSLRAEDTAVYYCARGGYGHTMDYWGQGTLVTVSS

SEQ ID NO: 393 - protein, 117 aa, synthetic construct
EVQLVESGGGLVQPGGSLRLSCAVSGFSLTTYGVHWVRQAPGKGLEWVAVIWTGGSTNYNSALMSRFTISKDDSKNTAYLQMNSLRAEDTAVYYCAKGGYGHTMDYWGQGTLVTVSS

SEQ ID NO: 394 - protein, 117 aa, synthetic construct
EVQLVESGGGLVQPGGSLRLSCAVSGFSLTTYGVHWVRQAPGKGLEWVAVIWTGGSTNYNSALMSRFTISKDDSKSTAYLQMNSLRAEDTAVYYCAKGGYGHTMDYWGQGTLVTVSS

SEQ ID NO: 395 - protein, 117 aa, synthetic construct
EVQLVESGGGLVQPGGSLRLSCAVSGFSLTTYGVHWVRQAPGKGLEWVAVIWTGGSTNYNSALMSRVTISKDDSKNTVYLQMNSLRAEDTAVYYCAKGGYGHTMDYWGQGTLVTVSS

SEQ ID NO: 396 - protein, 117 aa, synthetic construct
EVQLVESGGGLVQPGGSLRLSCAVSGFSLTTYGVHWVRQAPGKGLEWVAVIWTGGSTNYNSALMSRFTISKDDSKNTVYLQMNSLRAEDTAVYYCAKGGYGHTMDYWGQGTLVTVSS

SEQ ID NO: 397 - protein, 117 aa, synthetic construct
EVQLVESGGGLVQPGGSLRLSCAVSGFSLTTYGVHWVRQAPGKGLEWVAVIWTGGSTNYNSALMSRATISKDDSKNTVYLQMNSLRAEDTAVYYCAKGGYGHTMDYWGQGTLVTVSS

SEQ ID NO: 398 - protein, 117 aa, synthetic construct
EVQLVESGGGLVQPGGSLRLSCAVSGFSLTTYGVHWVRQAPGKGLEWVAVIWTGGSTNYNSALMSRITISKDDSKSTVYLQMNSLRAEDTAVYYCARGGYGHTMDYWGQGTLVTVSS

SEQ ID NO: 399 - protein, 117 aa, synthetic construct
EVQLVESGGGLVQPGGSLRLSCAVSGFSLTTYGVHWVRQAPGKGLEWVAVIWTGGSTNYNSALMSRATISKDDSKSTVYLQMNSLRAEDTAVYYCAKGGYGHTMDYWGQGTLVTVSS

SEQ ID NO: 400 - protein, 117 aa, synthetic construct
EVQLVESGGGLVQPGGSLRLSCAVSGFSLTTYGVHWVRQAPGKGLEWVAVIWTGGSTNYNSALMSRFTISKDDSKSTVYLQMNSLRAEDTAVYYCAKGGYGHTMDYWGQGTLVTVSS

SEQ ID NO: 401 - protein, 117 aa, synthetic construct
EVQLVESGGGLVQPGGSLRLSCAVSGFSLTTYGVHWVRQAPGKGLEWVAVIWTGGSTNYNSALMSRFTISKDNSKNTVYLQMNSLRAEDTAVYYCAKGGYGHTMDYWGQGTLVTVSS

SEQ ID NO: 402 - protein, 117 aa, synthetic construct
EVQLVESGGGLVQPGGSLRLSCAVSGFSLTTYGVHWVRQAPGKGLEWVAVIWTGGSTNYNSALMSRTTISKDDSKNTVYLQMNSLRAEDTAVYYCAKGGYGHTMDYWGQGTLVTVSS

SEQ ID NO: 403 - protein, 117 aa, synthetic construct
EVQLVESGGGLVQPGGSLRLSCAVSGFSLTTYGVHWVRQAPGKGLEWVAVIWTGGSTNYNSALMSRVTISKDNSKNTVYLQMNSLRAEDTAVYYCAKGGYGHTMDYWGQGTLVTVSS

SEQ ID NO: 404 - protein, 117 aa, synthetic construct
EVQLVESGGGLVQPGGSLRLSCAISGFSLTTYGVHWVRQAPGKGLEWVAVIWTGGSTNYNSALMSRTTISKDDSKNTVYLQMNSLRAEDTAVYYCAKGGYGHTMDYWGQGTLVTVSS

SEQ ID NO: 405 - protein, 117 aa, synthetic construct
EVQLVESGGGLVQPGGSLRLSCAISGFSLTTYGVHWVRQAPGKGLEWVAVIWTGGSTNYNSALMSRFTISKDDSKSTAYLQMNSLRAEDTAVYYCAKGGYGHTMDYWGQGTLVTVSS

SEQ ID NO: 406 - protein, 117 aa, synthetic construct
EVQLVESGGGLVQPGGSLRLSCAISGFSLTTYGVHWVRQAPGKGLEWVAVIWTGGSTNYNSALMSRFTISKDDSKNTVYLQMNSLRAEDTAVYYCAKGGYGHTMDYWGQGTLVTVSS

SEQ ID NO: 407 - protein, 117 aa, synthetic construct
EVQLVESGGGLVQPGGSLRLSCAISGFSLTTYGVHWVRQAPGKGLEWVAVIWTGGSTNYNSALMSRFTISKDNSKNTVYLQMNSLRAEDTAVYYCAKGGYGHTMDYWGQGTLVTVSS

SEQ ID NO: 408 - protein, 117 aa, synthetic construct
EVQLVESGGGLVQPGGSLRLSCAISGFSLTTYGVHWVRQAPGKGLEWVAVIWTGGSTNYNSALMSRFTISKDNSKNTVYLQMNSLRAEDTAVYYCARGGYGHTMDYWGQGTLVTVSS

SEQ ID NO: 409 - protein, 117 aa, synthetic construct
EVQLVESGGGLVQPGGSLRLSCAISGFSLTTYGVHWVRQAPGKGLEWVAVIWTGGSTNYNSALMSRVTISKDNSKNTVYLQMNSLRAEDTAVYYCAKGGYGHTMDYWGQGTLVTVSS

SEQ ID NO: 410 - protein, 117 aa, synthetic construct
EVQLVESGGGLVQPGGSLRLSCAISGFSLTTYGVHWVRQAPGKGLEWVAVIWTGGSTNYNSALMSRVTISKDDSKNTVYLQMNSLRAEDTAVYYCAKGGYGHTMDYWGQGTLVTVSS

SEQ ID NO: 411 - protein, 117 aa, synthetic construct
EVQLVESGGGLVQPGGSLRLSCAISGFSLTTYGVHWVRQAPGKGLEWVAVIWTGGSTNYNSALMSRFTISKDNSKNTAYLQMNSLRAEDTAVYYCAKGGYGHTMDYWGQGTLVTVSS

SEQ ID NO: 412 - protein, 117 aa, synthetic construct
EVQLVESGGGLVQPGGSLRLSCAISGFSLTTYGVHWVRQAPGKGLEWVAVIWTGGSTNYNSALMSRTTISKDNSKNTVYLQMNSLRAEDTAVYYCAKGGYGHTMDYWGQGTLVTVSS

SEQ ID NO: 413 - protein, 117 aa, synthetic construct
EVQLVESGGGLVQPGGSLRLSCAISGFSLTTYGVHWVRQAPGKGLEWVAVIWTGGSTNYNSALMSRFTISKDDSKNTAYLQMNSLRAEDTAVYYCAKGGYGHTMDYWGQGTLVTVSS

SEQ ID NO: 414 - protein, 117 aa, synthetic construct
EVQLVESGGGLVQPGGSLRLSCAVSGFSLTTYGVHWIRQAPGKGLEWVAVIWTGGSTNYNSALMSRTTISKDDSKNTVYLQMNSLRAEDTAVYYCAKGGYGHTMDYWGQGTLVTVSS

SEQ ID NO: 415 - protein, 7 aa, synthetic construct
GATNLAD

SEQ ID NO: 416 - protein, 16 aa, synthetic construct
  VARIANT 1: note = X is S, T, or Y
  VARIANT 8: note = X is A, I, K, L, M, N, P, S, T, V, or W
  VARIANT 9: note = X is H, L, N, S, or W
XISYDGNXXYNPSLKN